(19) United States
(12) Patent Application Publication
(10) Pub. No.: US 2022/0218256 A1
(43) Pub. Date: Jul. 14, 2022

(54) SYSTEMS AND METHODS FOR A PERSONAL MEDICAL MONITORING DEVICE

(71) Applicant: Arvind Thiagarajan, Santa Clara, CA (US)

(72) Inventor: Arvind Thiagarajan, Santa Clara, CA (US)

(21) Appl. No.: 17/570,346

(22) Filed: Jan. 6, 2022

Related U.S. Application Data
(60) Provisional application No. 63/135,565, filed on Jan. 9, 2021.

(51) Int. Cl. A61B 5/282; A61B 5/00; A61B 5/26; A61B 5/263

(52) U.S. Cl. CPC A61B 5/282; A61B 5/0006; A61B 5/26; A61B 5/263; A61B 2560/0242; A61B 5/6823; A61B 5/6826; A61B 5/742; A61B 2562/04; A61B 5/7203

(57) ABSTRACT

Systems and methods for a medical device are provided. The handheld medical device includes a housing including a front and rear side. A diaphragm is located in the center of the rear side of the housing. Three or less rear electrodes, configured to collect electromagnetic signals from the chest region of a patient, are located in a semi-circular in shape that encircles the diaphragm on the rear side of the housing. Two front electrodes located on the front side of the housing collect signals from the left and right index fingers of the patient. A screen is located between the two front electrodes. The device may include a transmitter, in some cases a Bluetooth module, for coupling the medical device to a user device. The user device includes an application that receives the signals and performs analysis on them. The five electrode inputs are used to calculate seven ECG channels.

### CROSS REFERENCE TO RELATED APPLICATION

The non-provisional application claims priority to U.S. Provisional Application No. 63/135,565 filed Jan. 9, 2021, entitled “HEALTHYU, AN INTELLIGENT ALL-IN-ONE REMOTE PATIENT MONITOR WITH INTEGRATED ECG, STETHOSCOPE AND VITAL CLINICAL PARAMETERS”, which application is incorporated herein in its entirety by this reference.

### BACKGROUND

The present invention relates in general to the field of medical devices and more specifically, to a personal device for cardiac and pulmonary monitoring and diagnosis. Such systems and methods are useful for assisting an individual to keep track of their health, and identify potentially dangerous pathologies before they become life threatening conditions.

Cardiac and pulmonary health is a major contributor to the overall health of a population. For example, according to the American Lung Association, just over six percent of the population suffers from Chronic Obstructive Pulmonary Disease (COPD), which is just a single lung pathology. Additionally, cardiac disease (particularly heart attacks and failures) is the leading cause of death in the United States, and costs society over $360 billion per year.

The ability to track cardiac and pulmonary disease (or per-disease states) can assist in reducing the rates of negative outcomes, and overall cost. Unfortunately, tracking cardiac functioning and lung activity typically takes specialized equipment not typically found outside a doctor's office. Further, interpretation of the results of these diagnostic tools usually requires a clinician. As such, an individual attempting to track their cardiac and/or pulmonary health needs to regularly see the doctor, which is costly, time consuming, and usually not feasible to do on as regular a basis as would be ideal.

Recently, more portable devices, aimed at in-home usage, have become available to consumers. These systems however generally suffer from sub-optimal resolution/sensitivity, poor analysis, and difficulty to use properly.

As such, the existing systems used for cardiac and/or pulmonary health classification are woefully inadequate for everyday usage, in-home, and without the need of a clinician present. It is therefore apparent that an urgent need exists for systems and methods for improved cardiac and/or pulmonary activity diagnosis in an easy-to-use device designed for personal use. Such systems and methods are designed to provide the user with improved visualization and diagnosis of potentially life-threatening conditions.

### SUMMARY

The present systems and methods relate to improving classification of cardiac and/or pulmonary activity with a personal, easy-to-use device. Such systems and methods enable improvements in the diagnosis of possible pathologies, as well as providing insights into the health and wellbeing of the user.

In some embodiments, the handheld medical device 160 includes a housing including a front and rear side. A diaphragm is located in the center of the rear side of the housing. Three rear electrodes, configured to collect electromagnetic signals from the chest region of a patient, are located in a semi-circular in shape that encircles the diaphragm on the rear side of the housing. Two front electrodes located on the front side of the housing collect signals from the left and right index fingers of the patient. A screen is located between the two front electrodes.

In some embodiments, the electrodes are brass plated in nickel, which are then gold plated to increase conductivity. The device may include a transmitter, in some cases a Bluetooth module, for coupling the medical device 160 to a user device. The user device includes an application that receives the signals, and performs analysis on them. In some embodiments, the chest electrodes may be labeled as electrode 1 (E1) electrode 2 (E2) and electrode 3 (E3). The front finger electrode are the left finger electrode (LA) and the right finger electrode (RA). These five inputs are used to calculate seven ECG channels. These are calculated as the first channel (CH1) is the generated by the LA minus RA (LA−RA). The second channel (CH2) consists of Electrode 2 minus RA (E2−RA). Channel 3 (CH3) is channel 2 minus channel 1 (CH2−CH1). Channel 4 (CH4) is the negative of channel 1 plus channel 2 divided by 2 (−(CH1+CH2)/2). Channel 5 (CH5) is channel 1 minus channel 2 divided by 2 (CH1−CH2/2). Channel 6 (CH6) is channel 2 minus channel 1 divided by 2 (CH2−CH1/2). Channel 7 (CH7) is Electrode 1 minus WTC (E1−WTC), where the WTC is the RA, LA and Electrode 2 signals added together and then divided by three ((RA+LA+E2)/3).

In addition to collecting the ECG signals, the device collects phonocardiogram (PCG) data from the diaphragm. Another microphone may pick up ambient noises to allow for noise cancellation. In some embodiments, the system may include an optical sensor and temperature sensor on the front side of the housing below the left and right electrodes. These sensors come in contact with the user's left and right middle fingers as the device is held to the chest, and collect data related to body temperature, blood oxygenation, pulse rate, blood pressure and glucose levels. Additionally, in some cases, there may be other sensors present, such as conductivity sensors and chemical sensors for collecting other physiological data (hormone levels for example).

The application on the user device may be coupled, in some embodiments, to a backend system via a network (generally the internet). This backend system may perform analysis on the collected data and may flag when there are identified issues or pathologies. When identified, the system may send an urgent alert to a clinician for review of the data, and if needed arrangement for the patient to come into a clinician's office, or in more urgent situations, directly to the hospital.

The backend system may rely upon basic rule-based analysis of signals to generate alerts or may leverage advanced machine learning algorithms in order to identify possible pathologies. In some embodiments, the application on the user device may perform some, or all, of this analysis when sufficient computing resources are available. In which case, the user device may directly engage with the clinician's system.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 is an example block diagram of a system for cardiac and/or pulmonary monitoring, in accordance with some embodiment;

FIG. 2 is an example block diagram for the HealthyU medical device, in accordance with some embodiment;

FIGS. 3A-3E are example illustrations of the HealthyU medical device exterior, in accordance with some embodiment;

FIG. 4A is an illustration of an example process for the operation of the medical device within the cardiac and/or pulmonary monitoring system, in accordance with some embodiments;

FIG. 4B is an illustration of an example process device initialization, in accordance with some embodiments;

FIG. 5 is an illustration of example ECG and PCG waveforms, in accordance with some embodiments;

FIG. 6 is an illustration of an example screenshot of the HealthyU device 7-channel recorded ECG waveforms, in accordance with some embodiments;

FIG. 7 is an illustration of an example screenshot of the HealthyU device recorded ECG and PCG waveforms, in accordance with some embodiments;

FIG. 8 is an illustration of an example screenshot of the HealthyU device first three channel recorded ECG waveforms, in accordance with some embodiments;

FIG. 9 is an illustration of an example screenshot of the HealthyU preview screen, in accordance with some embodiments;

FIG. 10 is an illustration of an example screenshot of the HealthyU device positioning and body posture, in accordance with some embodiments;

FIGS. 11-13 are illustrations of example screenshots of the HealthyU device settings screen, in accordance with some embodiments;

FIG. 14 is an illustration of an ECG waveform, in accordance with some embodiments;

FIG. 15 is an illustration of example HealthyU device positioning, in accordance with some embodiments;

FIGS. 16A and 16B are illustrations of computer systems capable of the backend diagnostic and analysis activity of the medical device system, in accordance with some embodiments;

FIGS. 17A and 17B provide renderings of the medical device screen, in accordance with some embodiments;

FIG. 18 provides a rendering of the battery level icons, in accordance with some embodiments;

FIG. 19 provides a rendering of the pairing screen of the application on the user device, in accordance with some embodiments;

FIG. 20 provides an example process of timer 2 operation, in accordance with some embodiments;

FIG. 21 provides an example process of timer 3 operation, in accordance with some embodiments;

FIG. 22 provides an example process of timer 4 operation, in accordance with some embodiments;

FIG. 23 provides an example process of timer updating, in accordance with some embodiments;

FIG. 24 provides an example process of battery management, in accordance with some embodiments;

FIG. 25 provides an example process of battery indication, in accordance with some embodiments;

FIG. 26 provides an example process of timer, firmware and battery updates, in accordance with some embodiments;

FIG. 27 provides an example process of signal acquisition, in accordance with some embodiments;

FIG. 28 provides an example process of initialization of the medical device 160, in accordance with some embodiments;

FIG. 29 provides an example process of ECG and PCG sampling, in accordance with some embodiments;

FIG. 30 provides an example process of medical device and user device pairing, in accordance with some embodiments;

FIG. 31 provides a rendering of a first version of a paired screen of the medical device, in accordance with some embodiments;

FIG. 32 provides a rendering of a second version of a paired screen of the medical device, in accordance with some embodiments;

FIG. 33 provides a rendering of a patient file management screen of the application on the user device, in accordance with some embodiments;

FIG. 34 provides a rendering of a patient file screen of the application on the user device, in accordance with some embodiments;

FIG. 35 provides a rendering of the top portion of a patient record screen of the application on the user device, in accordance with some embodiments;

FIG. 36 provides a rendering of the bottom portion of a patient record screen of the application on the user device, in accordance with some embodiments; and

FIG. 37 provides an illustration of the circuit diagram for the medical device 160, in accordance with some embodiments.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “always,” “only,” “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

The present invention relates to systems and methods for the monitoring of pulmonary and/or cardiac health. In particular, cardiac health is focused upon. It should be noted however that the medical device 160 may include many additional sensory capabilities that allow for complex and varied medical diagnoses that expand well beyond mere cardiac functioning. Thus, while this disclosure may center upon cardiac (and to a lesser degree pulmonary) activity, it is not intended to limit the scope of this disclosure or the functioning of the instant device to only monitor and analyze these particular fields of medicine.

General Description

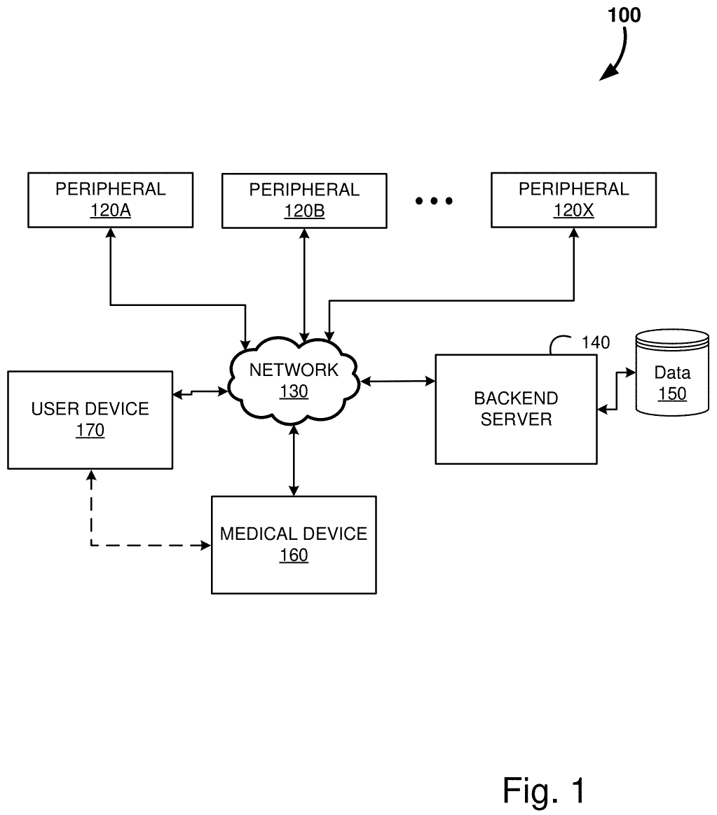

To facilitate discussions, FIG. 1 is an example medical monitoring system, shown generally at 100. In this example system a medical device 160 (also referred to as the HealthyU device) is coupled to a larger system via a network. The medical device 160 collects physiological data from a user (not illustrated). In some embodiments, the medical device 160 is a home healthcare electronic multiparameter system for patients' ≥10 Kg to capture, record and replay heart sounds (PCG) and electrocardiogram (ECG) rhythm. It is to be used by one patient at a time. Heart sounds (PCG) and 7-channel ECG rhythm are acquired and displayed simultaneously on an accompanying application software on a hand-held smart device 170. The waveforms can be recorded and sent to their clinician. The medical device 160 is a homecare device for use by lay operator, or clinician for tracking health parameters.

A user device 170 may connect to the medical device 160 either directly via a Bluetooth (or similar) connection, or via the network 130. The user device 170 may comprise a tablet, smartphone, computer, or virtually any device with a screen interface and some computational capacity.

Similarly, the medical device 160 may couple to one or more peripheral sensors 120a-x. This connectivity (when the peripheral device is present) may likewise be through the network 130 or direct through a Bluetooth or similar connection (not illustrated). The medical device 160 may also couple to a backend server 140 via the network 130. Since the medical device 160 is generally a relatively ‘light weight’ device, the backend server 140 allows for much deeper AI analysis of the collected physiological data, as well as providing connectivity for clinicians for review of the collected data (not illustrated). The backend server 140 includes data stores 150 for saving the collected medical data.

In most cases the network is comprised of a cellular network and/or the internet. However, it is envisioned that the network includes any wide area network (WAN) architecture, including private WAN's, or private local area networks (LANs) in conjunction with private or public WANs.

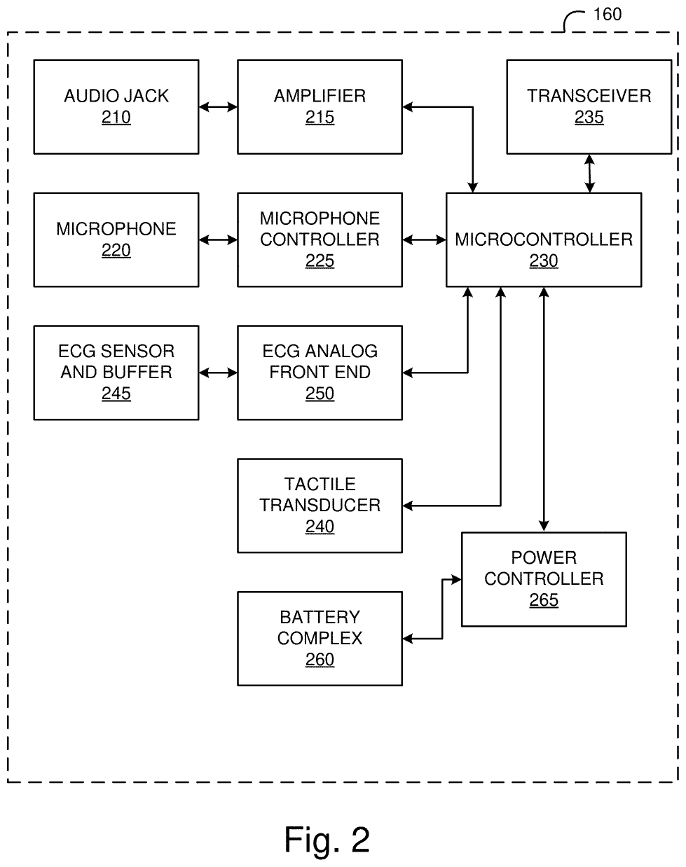

Turning to FIG. 2, a more detailed view of the medical device 160. The heart of the medical device 160 includes a microcontroller unit 230 which couples the other elements of the medical device 160 together, and provides the local processing of collected data. The microcontroller 230 may include or couple to a local memory (not illustrated) for maintaining user settings and caching of the collected data.

In some particular embodiments, the microcontroller is a flash microcontroller based on the high performance 32-bit ARM Cortex-M3 RISC processor. It operates at a maximum speed of 84 MHz and features up to 512 Kbytes of Flash and up to 100 Kbytes of SRAM. The peripheral set includes a Highspeed USB Host and Device port with embedded transceiver, an Ethernet MAC, 2 CANs, a Highspeed MCI for SDIO/SD/MMC, an External Bus Interface with NAND Flash Controller (NFC), 5 UARTs, 2 TWIs, 4 SPIs, as well as a PWM timer, 3-channel general-purpose 32-bit timers, a low-power RTC, a low power RTT, 256-bit General Purpose Backup Registers, a 12-bit ADC. Features of one embodiment of the microcontroller are found in Table 1:

TABLE 1
Microcontroller Features

| FEATURES | MICROCONTROLLER |
|---|---|
| Flash | 2 × 256 Kbytes |
| SRAM | 64 + 32 Kbytes |
| Package | LQFP144, LFBGA144 |
| Number of PIOs | 103 |
| EMAC | MII/RMII |
| External Bus Interface | 16-bit data, 8 chip selects, 23-bit address |
| 12-bit ADC | 16 ch |
| 12-bit DAC | 2 ch |
| USART/UART | 3/2 |
| SPI (Serial Peripheral | 1 SPI controller, 4 chip selects + |
| Interface) | 3 USART with SPI mode |
| 32-bit Timer | 9 ch |

The microcontroller architecture is specifically designed to sustain high-speed data transfers. It includes a multi-layer bus matrix as well as multiple SRAM banks, PDC and DMA channels that enable it to run tasks in parallel and maximize data throughput. The device operates at 3.3V and is available in 144-lead LQFP, LFBGA packages. In other embodiments, the user device may include the memory needed to store these pieces of information. An LCD or other display type that is driven by the microcontroller 230 is present on the device surface. The display allows the medical device 160 to provide battery indications, pairing instructions, and even collected signal data.

The microcontroller 230 can provided outbound audio via an amplifier 215 coupled to an audio jack 210. A transceiver 235 allows the medical device 160 to connect to the user device directly, or through the network. In some embodiments, the medical device 160 is incapable of direct connectivity with the network, in these situations, the medical device 160 couples directly with the user device (by Bluetooth or similar protocol) and the user device in turn provides connection to the internet or cellular network.

The medical device 160 also includes one or more microphones 220, which couples to the microcontroller 230 via a microphone controller 225. The microphone controller 225, when coupled to multiple microphones, may allow for directional or doppler type of analysis. Phonocardiogram or PCG is the recording of all the sounds made by the heart during a cardiac cycle. It is a plot of high-fidelity recording of the heart sounds and made by the heart. Additionally, a secondary microphone may be utilized to recognize ambient noise for the purposes of noise cancelation.

ECG sensors and buffers 245 couple to the ECG analog front end 250. In some embodiments there are five ECG sensor pads. The medical device 160 incorporates all features commonly required in portable, low-power medical, sports, and fitness electrocardiogram (ECG) applications. With high levels of integration and exceptional performance, the medical device 160 enables the creation of scalable medical instrumentation systems at significantly reduced size, power and overall cost.

A battery complex 260 includes a rechargeable Lithium Polymer battery, which is used for powering the medical device 160. In some specific embodiment, the battery nominal voltage is 3.7V. The power supply controller 265 controls the power supplies of the processor and peripherals via Voltage regulator control. In some specific embodiment, the power controller has its own reset circuitry and is clocked by the 32 kHz slow clock generator. The reset circuitry is based on a zero-power power-on reset cell. The zero-power power-on reset allows the power controller to start properly.

In some embodiments, the power controller is a low-noise, linear regulators that deliver up to 500 mA of output current with only 10.5 μVRMS of output noise from 10 Hz to 100 kHz. These regulators maintain ±1% output accuracy over a wide input voltage range, requiring only 100 mV of input-to-output headroom at full load. The 365 μA no-load supply current is independent of dropout voltage. It includes the programmable output soft-start rate, output overcurrent, and thermal overload protection.

A tactile transducer 240 provides vibration or other tactile output to the user. For example, once a sufficient sampling for the ECG or PCG is collected, the user may be notified by a slight vibration of the device.

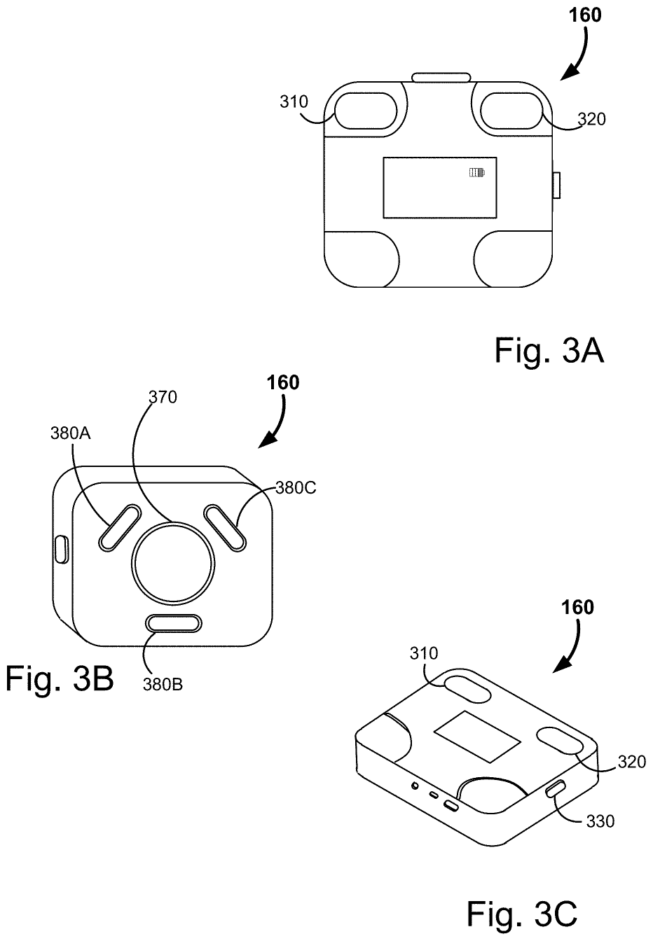

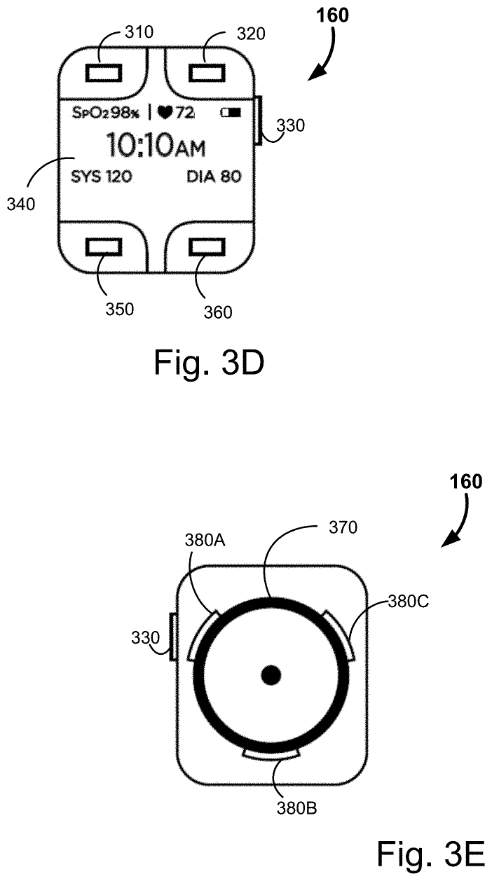

FIGS. 3A to 3E provide illustrations of the device 160 from the front, back and side, respectively. In FIG. 3A, the first two ECG electrodes are seen 310 and 320. These include left and right index finger pads which collect the left arm (LA) and right arm (RA) signals, respectively. The device may also include a screen, additional finger rests, power button, and other convenience features (such as a lanyard hook or the like). On the rear side of the device, shown at FIG. 3B, the chest placement ECG sensors are seen (E1, E2 and E3) 380A-C respectively. These three electrodes operate with the LA and RA signals to generate the seven channel ECG analysis. One or more diaphragms 370 collect the chest sound waves. On FIGS. 3C and 3E, the connectivity ports for the device are seen. These may include a charging port (which may also be a data transfer port, in some embodiments), heart sound outputs (audio jack), and a charge indicator. On FIG. 3D, the enhanced screen 340 content is seen. This includes where the blood oxygenation, heart rate, battery, time, and blood pressure being displayed. This version of the medical device 160 also includes the front right arm electrode 310 and left arm electrode 320, as well as an optical sensor 350 for collecting the pulse rate, oxygenation, and blood pressure. A secondary sensor 360 includes an array of sensors, including at least one of a temperature sensor, chemical sensors, and conductivity sensor. The power button 330 can also be seen. On FIG. 3E, the diaphragm 370 is seen centrally located, surrounded by semicircular chest electrodes 380A-C.

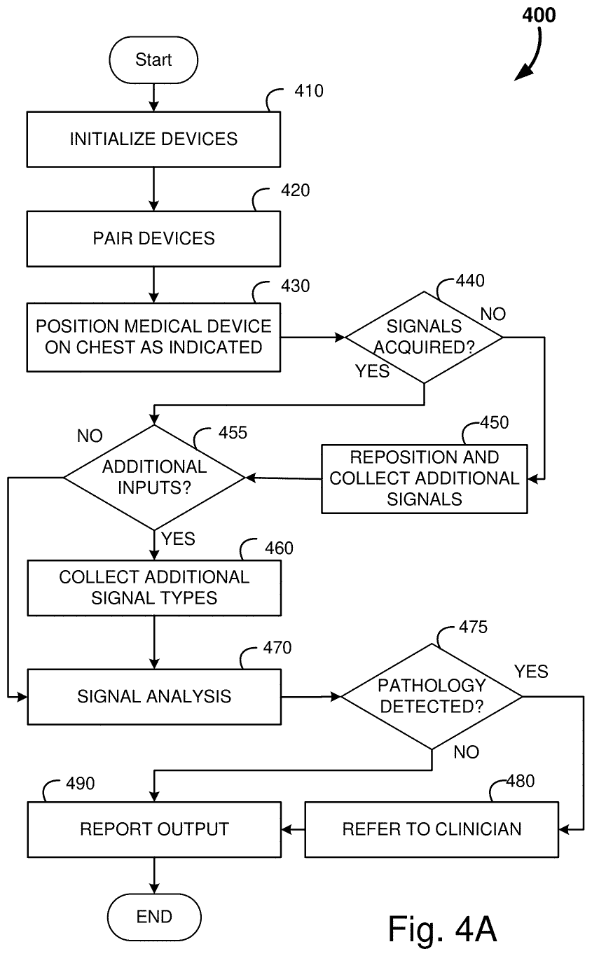

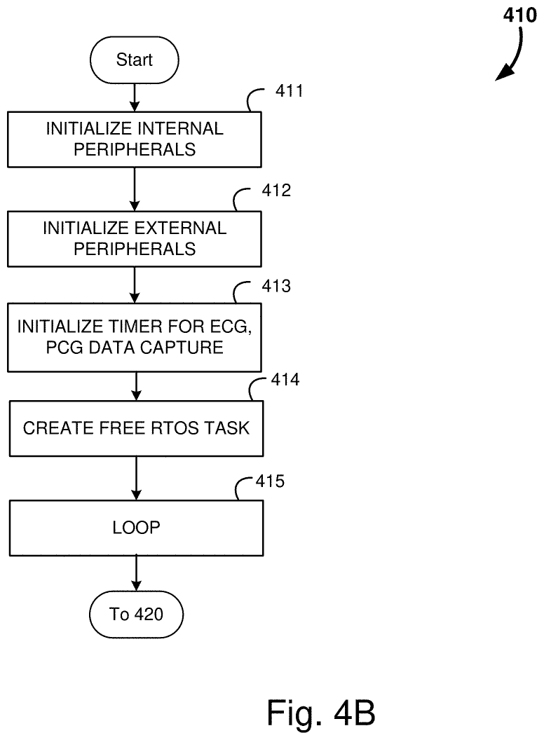

Turning now to FIG. 4A, an example process for the operation of the medical monitoring system is provided, seen generally at 400. In this example process, the devices are initialized (at 410). FIG. 4B provides this initialization process in greater detail. Firstly, the internal peripherals are initialized (at 411). For example, when the system is powered on, it can initialize the ports, pins and interfaces. Likewise, the external peripherals, if present, are initialized (at 412).

The timer for ECG and PCG data capture are likewise initialized (at 413). Clock inputs may include a Low Power 32.768 KHz Slow Clock Oscillator with bypass mode, a low power RC oscillator, a 3 to 20 MHz Crystal or Ceramic Resonator-based Oscillator, which can be bypassed, a 480 MHz UTMI PLL, providing a clock for the USB High Speed Controller, and a 96 to 192 MHz programmable PLL (input from 8 to 16 MHz), capable of providing the clock MCK to the processor and to the peripherals. A Watchdog Timer can be used to prevent system lock-up if the software becomes trapped in a deadlock. It features a 12-bit down counter that allows a watchdog period of up to 16 seconds (slow clock at 32 kHz). It can generate a general reset or a processor reset only. In addition, it can be stopped while the processor is in debug mode or idle mode. The reset time of watchdog timer is 1 sec. It can continuously monitor the microcontroller program, to update the ECG or PCG data as per data protocol format and sends to Bluetooth low energy when it is formatted.

Next, a free RTOS task is created (at 414), and the system loops (at 415). Free RTOS is a real time scheduler for microcontroller applications to meet their hard real-time requirements. It allows microcontroller applications to be organized as a collection of independent tasks to be executed based on priority. The functions of Free RTOS make the round robin in the case of tasks with the same priority.

Returning to FIG. 4A, after device initialization, the medical device 160 is paired (at 420) with the user device 170. As discussed previously, this may be a direct Bluetooth (or equivalent) connection, or a connection via the network.

The user is then given a series of instructions about positioning of the medical device 160. The medical device 160 is thus positioned against the user's chest in various positions. The three backside electrodes contact the skin, and with the inputs from the LA and RA sensors, generate the seven channels for the ECG. If there is not a proper signal(s) being collected (at 440) the user is prompted to reposition the device, and collect additional signals (at 450). A decision is then made if additional inputs are needed (at 455). These additional inputs typically include sound collection (for PCG and pulmonary analysis), temperature and optical sensory data (for blood pressure, oxygen sensory purposes, blood pressure, glucose levels, red blood cell counts, and corroboration of pulse). In some embodiments, the device may include even more inputs, including skin conductivity measurements, and chemical analysis. Once these additional signals are collected (at 460), the signal analysis occurs (at 470). In some alternate embodiments, the various sensors, including the optical sensor, may be co-located with the index finger electrodes. In some embodiments, this allows for four distinct measurement locations (two at top locates with the electrodes, and two at the bottom finger placement locations). Having multiple sensors allows for redundancy if the signal is poor from another sensor (such as by poor finger placement), and allows averaging of signals to improve accuracy.

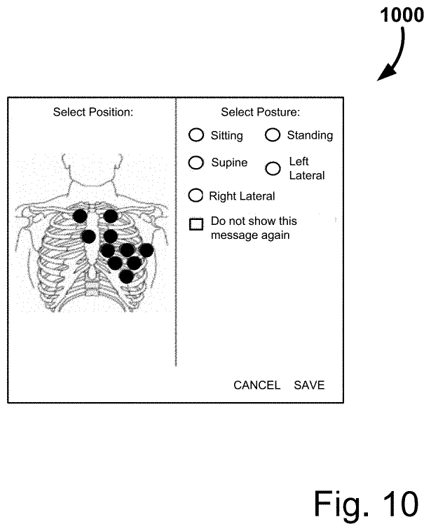

In the 5-Lead ECG shown in FIGS. 3A-3C, the medical device 160 uses a Common-Mode Detector to measure the common-mode of the system by averaging three voltage of input pins, and uses this signal in the right-leg drive feedback circuit. A Wilson Central Terminal is generated by the medical device 160 and is used as a reference to measure the chest electrode. The chip uses an external 4.096 MHz crystal oscillator to create the clock source for the device. The ECG signals are captured using 5 electrodes placed on circular board. Three circular shape electrodes Projected on Chest side and two finger electrode placed on display side to pick the ECG. The 5 electrodes are brass plated with nickel and then gold plated to increase conductivity. A high pass filter rectifies the signal capture by electrodes and a filtering circuit filters the signal and send it through SPI channel for digital signal processing. The five leads are used to generate a seven channel ECG signal. Using FIGS. 3A and 3B as a reference, the first channel (CH1) is the generated by the LA minus RA (LA−RA). The second channel (CH2) consists of Electrode 2 minus RA (E2−RA). Channel 3 (CH3) is channel 2 minus channel 1 (CH2−CH1). Channel 4 (CH4) is the negative of channel 1 plus channel 2 divided by 2 (−(CH1+CH2)/2). Channel 5 (CH5) is channel 1 minus channel 2 divided by 2 (CH1−CH2/2). Channel 6 (CH6) is channel 2 minus channel 1 divided by 2 (CH2−CH1/2). Channel 7 (CH7) is Electrode 1 minus WTC (E1−WTC), where the WTC is the RA, LA and Electrode 2 signals added together and then divided by three ((RA+LA+E2)/3). The placement of electrodes in human body, while diagnosing is very important for getting a good ECG signal. Electrode location is shown generally at 1000 of FIG. 10.

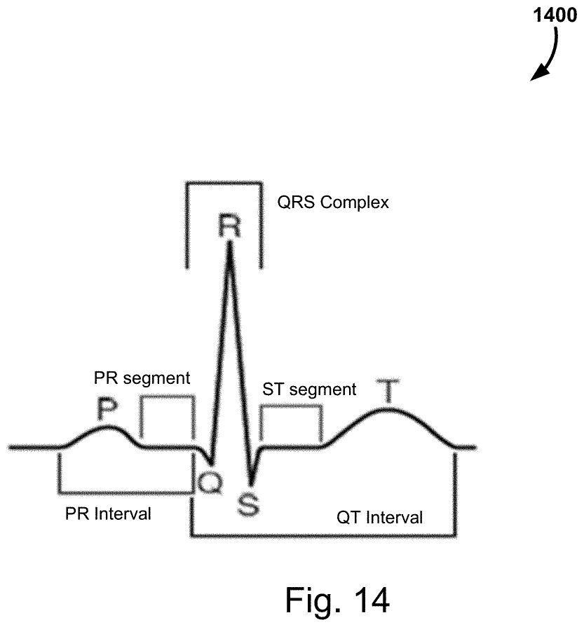

For reference, an example ECG sample is provided for informational purposes, seen at 1400 of FIG. 14. The P-wave is generally less than or equal to 0.11 seconds, the PR interval is between 0.12 to 0.20 seconds. The QRS complex is less than or equal to 0.12 seconds. The amplitude of this wave should be between 0.5 mV and 3.0 mV. The ST segment is between 0.08 and 0.12 seconds. Lastly, the QT interval is less than or equal to 0.4 seconds for males, and 0.44 for females. The regular sinus rhythm is between 60-100 beats per minute. Any parameters measured that are substantially outside these ranges are flagged by the system for analysis by a clinician or via an AI analyzer. Substantially outside the range, may include values that are greater than one, two or three standard deviations from these normative values.

In addition to the ECG signal processing, a phonocardiogram (PCG) signal is likewise acquired. A phonocardiogram (or PCG) is the recording of all the sounds made by the heart during a cardiac cycle. It is a plot of high-fidelity recording of the sounds made by the heart. The heart sound is distinguished as two components: the first heart sound (S1) and the second heart sound (S2). S1 is due to closure of mitral and tricuspid valves which permit the flow of blood from atria into the ventricles. The duration of S1 is 50-100 msec, with a frequency of 30-45 Hz. S1 also has a greater amplitude than S2. The S1 is best heard at the apex of mid pericardium. The S2 occurs at the end of ventricular systole due to closure of semilunar valves (aortic and pulmonary aortic valves) in the arteries leading out of the ventricles. S2 has a duration of 25-50 msec, and a frequency of 50-70 Hz. S2 is best heard in the aortic and pulmonary areas.

In some embodiments, the main microphone is a diaphragm. The microphone is a PVC sleeve with a polycarbonate diaphragm. The microphone input is sent to a printed circuit board (PCB) analog front end (AFE). This PCB AFE is a low-power rail-to-rail input/output operational amplifier specifically designed for portable applications. The input common-mode voltage range extends beyond the supply rails for maximum dynamic range in low-voltage systems. The amplifier output has rail-to-rail performance with high-output-drive capability, solving one of the limitations of older rail-to-rail input/output operational amplifiers. This rail-to-rail dynamic range and high output drive make the PCB AFE ideal for buffering analog-to-digital converters. The operational amplifier has 6.4 MHz of bandwidth and 1.6 V/μs of slew rate with only 500 μA of supply current, providing good ac performance with low power consumption. Three members of the family offer a shutdown terminal, which places the amplifier in an ultralow supply current mode (IDD=0.3 μA/ch). While in shutdown, the operational-amplifier output is placed in a high-impedance state. DC applications are also well served with an input noise voltage of 11 nV/✓Hz and input offset voltage of 100 μV.

The PCB AFE is then coupled to an audio amplifier 215 which, in some embodiments, is a mono bridged audio power amplifier capable of delivering power into a 3Ω load with less than 10% THD. A secondary microphone provides signal of ambient noise in order to allow for noise cancellation.

Returning to FIG. 4A, after signal analysis, a determination is made whether a pathology has been detected or not (at 475). This pathology detection process may be as simple as detection of any ECG, PCG, heart rate, temperature, blood pressure, or other detected signal outside of a normative value. Alternatively, more complex algorithms designed for detection of pathologies may be employed. In some embodiments, this can also include leveraging of AI models that have been trained on large datasets of normal and pathology signals. These may include deep neural networks. These pathology detection processes may occur in the medical device 160 itself, in the user device, but most commonly on the backend server. This stems from the enhanced processing capabilities of the backend system.

If a pathology is detected (or in some instances where the patient is in need of more regular monitoring) the data may be transferred to a clinician for review (at 480). Lastly, the output of the system may be presented (at 490) either on the user device itself, on the medial device, or made available via the cloud (in conjunction with the backend server).

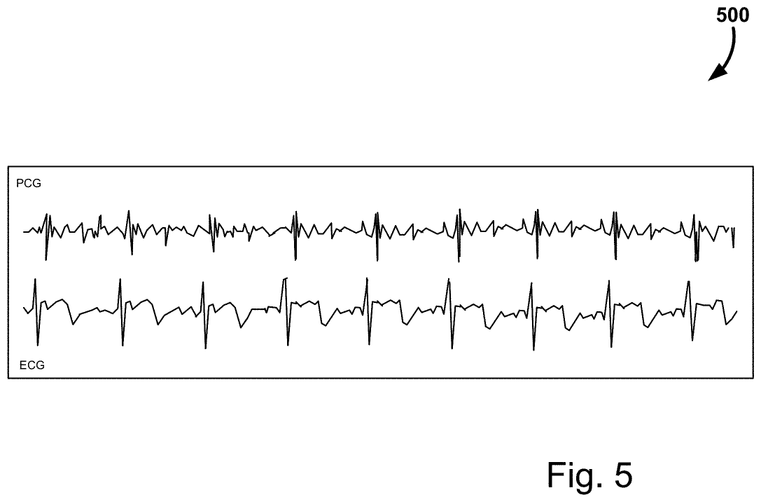

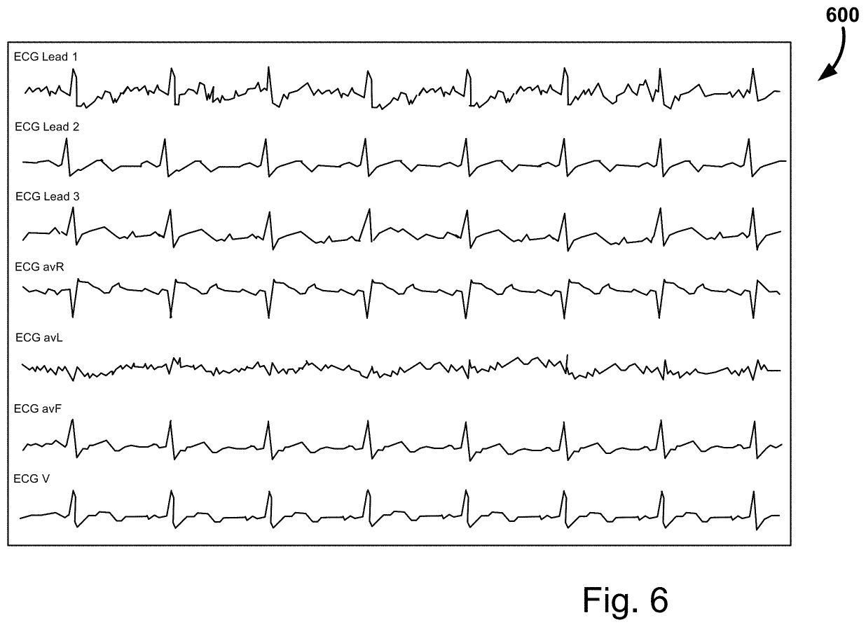

The outputs may appear as a PCG and ECG signal, as seen at 500 of FIG. 5. In this example raw output, the PCG signal is illustrated on the top, and a single ECG signal is presented on the bottom. In another embodiment, all seven ECG signals may be presented to the clinician or user, as seen at 600 of FIG. 6. Here the 7-channel ECG waveform (limb leads, chest leads and augmented leads) are displayed simultaneously in this screen.

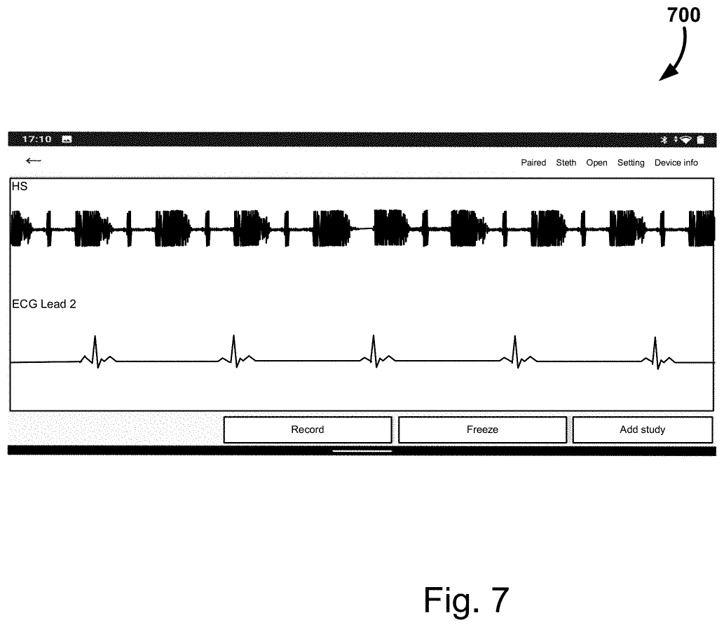

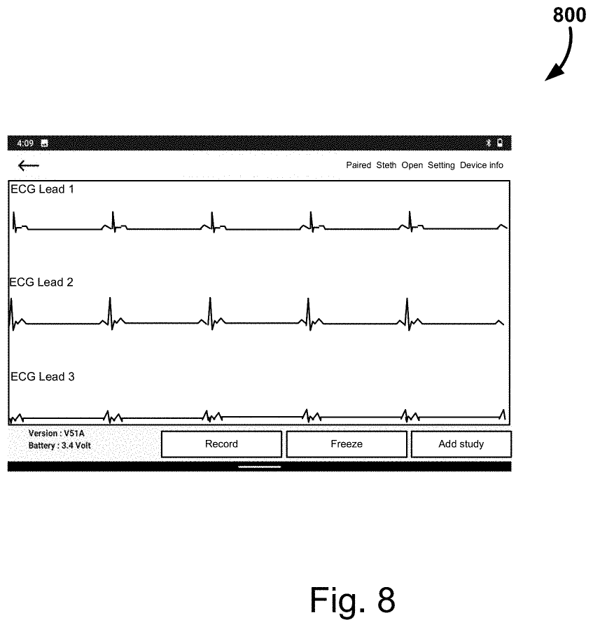

FIG. 7 illustrates another example signal output with a single PCG sample with a single ECG sample, shown generally at 700. Here is user or clinician has the option of recording the signals, freezing a portion of the signal for greater analysis, or adding a study to the sample. Similarly, FIG. 8 provides the first three ECG samples within the system's preview environment where it is possible to record samples, freeze them or add them to a study.

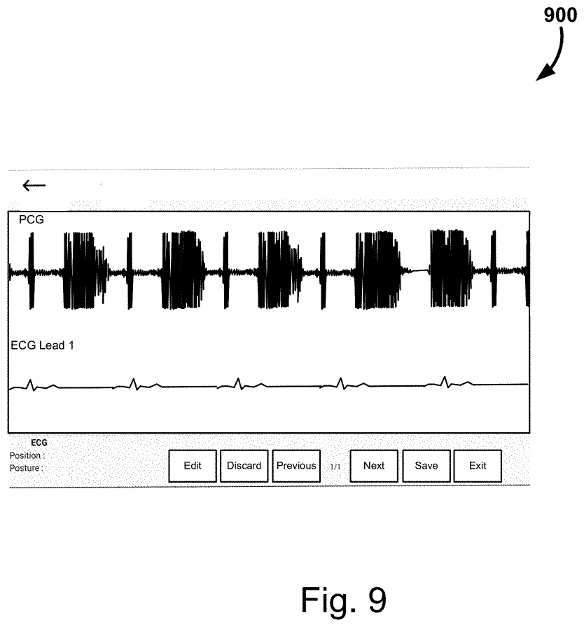

Once a set of samples is recorded for analysis, the data may become available on a screen such as the one found at 900 of FIG. 9. Here the sample may be edited, discarded when no longer needed and saved. The user may switch between available samples, and may exit at any time.

FIG. 10 provides a feedback screen where the user can select the position the medical device 160 was placed upon the body, as well as the position the user is in when using the device, shown generally at 1000. This is particularly helpful due to the fact that different postures and placements all yield differing results, and may analyze for different possible signs of pathologies. This selection information is useful for the clinician, as well as for any pathology detection software, in their analysis.

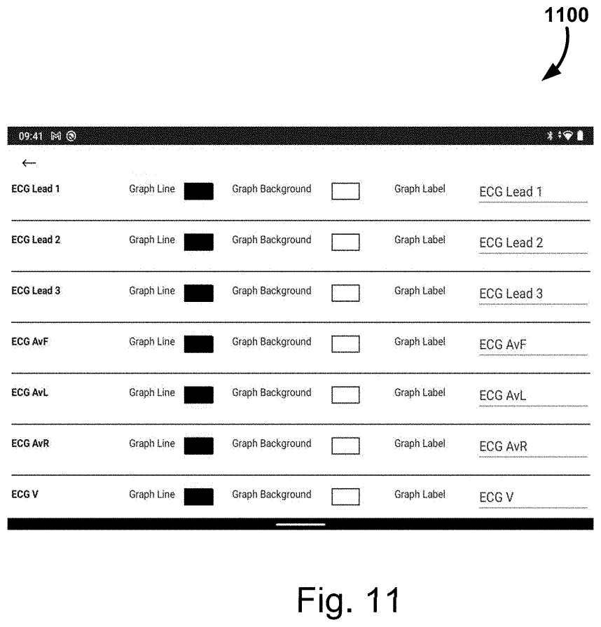

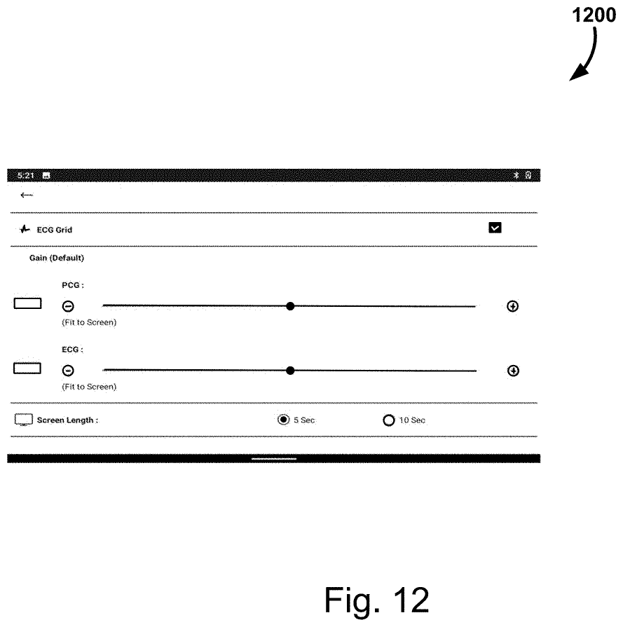

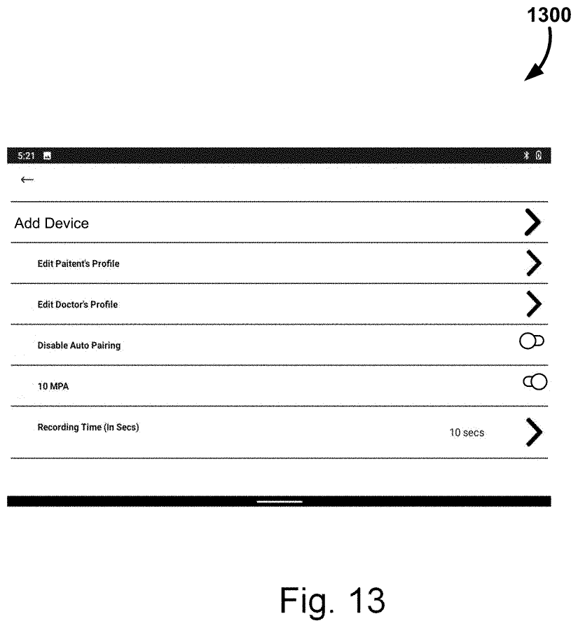

FIG. 11 provides an example screenshot of the settings of the various ECG signals, seen generally at 1100. Similarly, screen length and gains may be configured at a settings screen such as the one seen at 1200 of FIG. 12. A summary settings page, such as 1300 of FIG. 13, provides the ability to edit and pair devices, alter user and clinician profiles, alter recording times and the like.

### Particular Embodiments

Now that the general description of device structure and capabilities have been disclosed, a particular set of embodiments shall be disclosed for illustrative purposes. It should be noted that the particular limited sets of embodiments detailed in this sub-section are for illustrative purposes only, and are not intended to artificially limit the scope of the invention. As such, terms such as “must”, “can't” “will” and other such limiting language, is intended to only apply to the one instant embodiment being contemplated. Such restrictions are not intended to expand to other embodiments which may have a scope significantly broader than the instant substantiation.

As noted, HealthyU is a handheld device capable of sensing ECG and PCG using 3 chest electrodes and 2 finger electrodes, positioned against the chest. The HealthyU application is connected to HealthyU device via Bluetooth communication. HealthyU application displays the ECG and PCG for user or clinician observation. HealthyU system essential performance includes successful pairing of HealthyU device and HealthyU application using Bluetooth communication. ECG and PCG are monitored and displayed over this wireless communication. HealthyU application can be installed in any smart device that meets the minimum requirements, including: Android 9 and above, 2 GB RAM, 8 GB ROM, Screen Resolution 1280×800, Bluetooth 5.0, BLE (Bluetooth Low Energy), Wi-Fi, Global Positioning System (GPS), and an audio jack. Wherever the term device and app is mentioned, it refers to HealthyU device and HealthyU app respectively.

In particular embodiments, the medical device 160 should not be used on patients with cardiac pacemakers, defibrillators or other electronic implanted or wearable devices. Not used as sole basis for medication or treatment decisions, and not used during defibrillation, it is not defibrillator proof. In alternate embodiments, the device may rather include defibrillator functions that allow for defibrillation when cessation of cardiac function is detected.

When using the device, standard procedures for device positioning should be followed. Device positioning information is available in this manual and HealthyU application software. HealthyU uses Bluetooth low energy wireless data link. The Bluetooth range may be reduced when objects (walls, furniture, people, etc.) are between the HealthyU device and a paired mobile device. To improve Bluetooth connection, reduce the distance and ensure a line of sight between Healthy and mobile device consisting of HealthyU application.

In some embodiments, the device shall not be used for diagnostic purposes without clinician's consultation, and federal law restricts this device to sale to or on the order of a physician. Failure to follow the directions given in this manual could result in damage to the device and/or possible injury to the user/patient. Failure to follow operating and maintenance instructions, listed in the manual could result in malfunction of the device. The device is not recommended for use in the presence of equipment producing strong electromagnetic radiation/Magnetic Resonance Imaging (MM) and stacked device environment as it may affect the device functionality. Do not place the device on wet surfaces. Allowing the chest piece diaphragm to come into contact with liquids may affect the device functionality.

The device is not intended to be used on open wounds. Do not use any sharp pointed tool to reset the device. Do not expose the battery to a flame or excessive heat, immerse in or expose to water, short the terminals or disassemble the battery as doing so could damage the battery cause fire, injury or environmental contamination. Do not place heavy object on the device. Use only HealthyU accessories listed in this manual. Usage of any other accessories along with HealthyU device can compromise safety of the user. No special training or qualification is required to use the device. The HealthyU has no serviceable parts and does not require calibration. Do not open the device or perform any services. Failure to follow care and maintenance recommendations given in this manual could result in damage to the internal components of the HealthyU. Do not expose the device to a magnetic resonance (MR) environment. Conductive parts of electrodes and associated connectors for Type BF Applied Parts, including the neutral electrode, should not contact other conductive parts including earth.

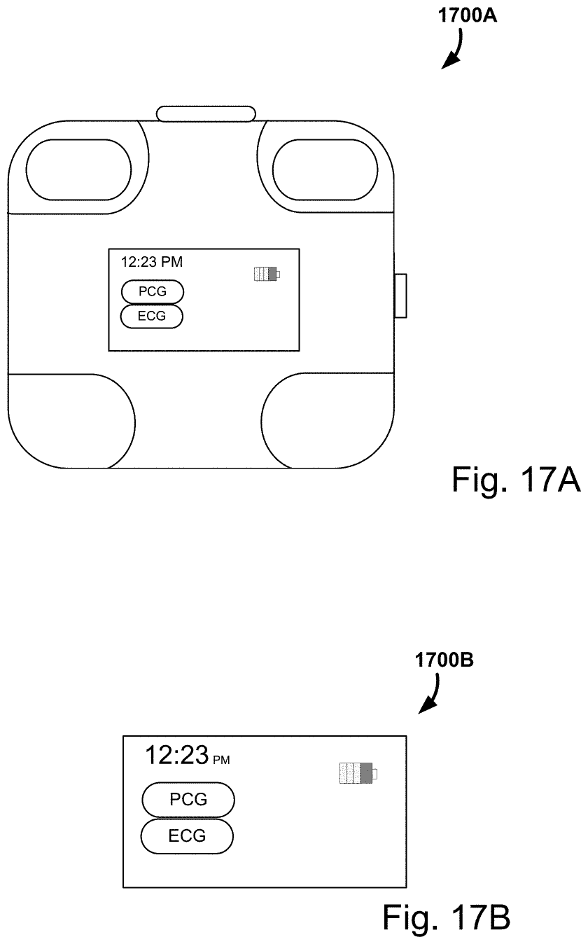

For operation, to switch the device ON, momentarily press the power button. Once the device is switched ON, the display may show the HD Medical logo followed by HealthyU® logo with firmware version number. To switch the device OFF, momentarily press the power button. The following functional screen seen at 1700A and 1700B of FIGS. 17A and 17B, respectively, may be displayed after device initialization. In this screen, time in 12 hour format, PCG icon with text, ECG icon with text and Battery status icon are displayed. Once this initialization screen is displayed, the device is ready for use.

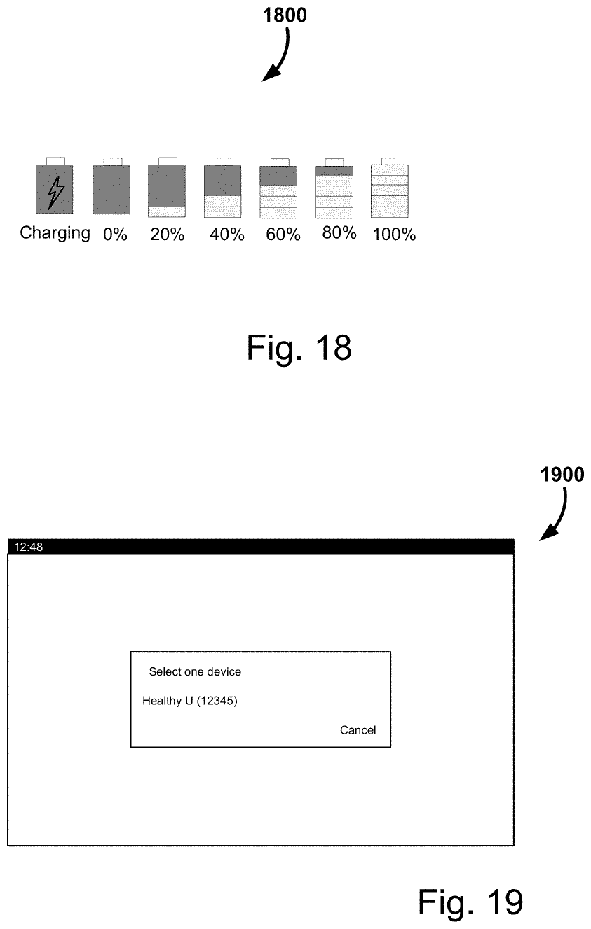

The battery level icon on the top right corner of the display screen indicates current battery charge level of the device. Stages of battery level icon are represented in FIG. 18, at 1800. The device is powered with a rechargeable Li-Polymer battery. To charge the device, connect the USB cable into the device charging port and into the charger provided. While connecting USB cable, if the device is in ON the following occurs: 1) Charging icon as shown in FIG. 18 is displayed, 2) Charging indicator may illuminate with amber color, and 3) “Do not use, while charging” message is displayed. Once the charger is connected, device displays warning message and automatically shuts itself down to disable device functionality and use. The medical device 160 cannot be used in charge mode. This protects user by preventing operation while it is connected to the AC power supply. Charging completion is checked by turning ON the device, the device displaying fully or 100% charged battery level icon as shown in FIG. 18, and the charging indicator LED is illuminate in green color. Li-Polymer batteries discharge continuously. The battery may be recharged periodically, at least every 3 to 4 months. This may ensure the battery doesn't fail prematurely.

ECG and PCG signals acquired by HealthyU device are displayed on the HealthyU application installed on the paired smart device. HealthyU application displays guidelines for device placement. Before proceeding with device placement, the HealthyU application on the user device should be paired with the HealthyU device. From the device placement till the completion of ECG & PCG recording, Bluetooth of the smart device should be ON and both HealthyU device and HealthyU application should be paired.

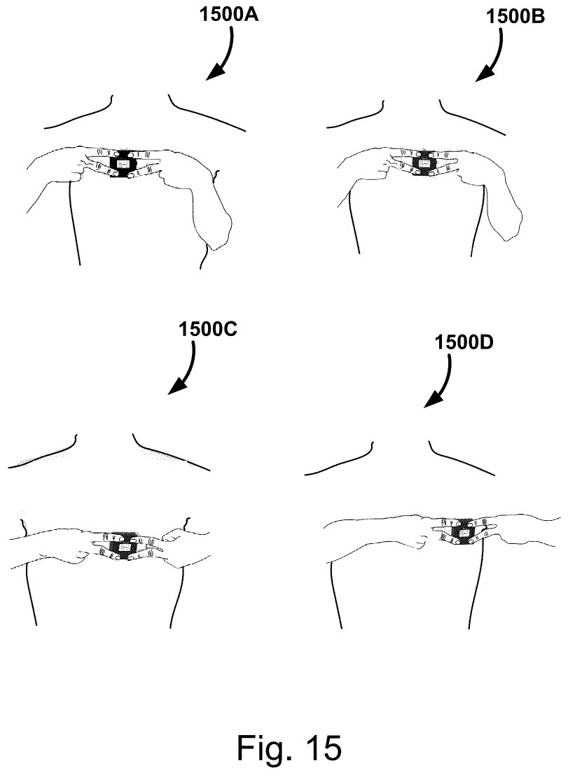

For operation, the user is to assume sitting/standing/supine/side-supine posture for ECG and PCG monitoring. The device is then held in the following manner: 1) with the lanyard hook facing upward direction, 2) with the three chest electrodes contacting chest region of user, 3) with the finger electrodes facing away from the body, and 4) with the smart display facing away from the body. Next, the device is placed in direct skin contact with the user. The device is then placed in at least four positions, as shown at 1500A, 1500B, 1500C and 1500D of FIG. 15. HealthyU application start screen also displays device placement information. Throughout the recording time users should place both index fingers on the upper slots and both middle fingers on the lower slots.

To use the device, the user should open the HealthyU application on the smart device. Smart device is any device of the user that meets the hardware requirements provided in table 1 above. Examples of smart devices: Mobile Phone, Tablet or Laptop. Next the user should pair HealthyU device and HealthyU application via Bluetooth. After Bluetooth pairing of device and application, HealthyU application may take user to the device placement guidelines followed by Steth screen display. Place the device directly on any one of the placement positions given in FIG. 15 for at least 10 seconds for heart sounds (PCG) and channel 2 electrocardiogram (ECG). Real time heart sounds (PCG) and channel 2 electrocardiogram (ECG) waveforms are displayed in the HealthyU application Steth Screen. Place the device directly on any one of the placement positions given in FIG. 15 for at least 10 seconds for 7-channel electrocardiogram (ECG). Real time 7 channel ECG signals are displayed in the HealthyU application's 7-channel screen. Ensure the ECG, PCG signal are in sync and displayed on the app screen by choosing the Steth mode in the application.

To record ECG & PCG, a RECORD button is present in both steth screen and 7-channel screen, which user has to manually select to initiate recording. Once recording duration is completed, user is taken to preview screen. In the preview screen, user can choose to SAVE or DISCARD the recorded ECG and PCG signals. Representation of Steth screen and 7-channel screen are given in FIGS. 5 and 6 respectively.

Use only HDI diaphragm with the device. Use of non□HDI diaphragms can result in faulty audio and display and possible analysis irregularities in any future detection/screening algorithms. USB Type C connector to USB 4-Pin Type-A Male connector can be used to connect the device to the charger. Length is 1 meter. The battery charger Input: 100-240V AC, 50/60 Hz, and Output: 5.0V DC, 1000 mA.

In some embodiment, the technical specifications of the device are as found in Table 2, below:

TABLE 2
Technical specifications

| | |
|---|---|
| Display | 1.14″ IPS TFT-LCD Color, 135 × 240 Pixels, |
| Battery | Rechargeable Li-polymer battery |
| | 1200 mAh 3.7 V |
| | 5 hours of continuous operation, when fully |
| | charged |
| Required interval | Battery cycle life is 300 charge cycles |
| for replacement | |
| of battery | |
| Battery charge time | 3 Hours (with fully depleted battery) |
| Charger | Input: 100-240 V AC, 50/60 Hz; Output: 5.0 V |
| | DC, 1000 mA. |
| Operating | 5° C. to 45° C. |
| temperatures | |
| Operating humidity | 10% to 95% relative humidity without |
| | condensation |
| Storage & transport | −25° C. to +70° C. |
| temperature | |
| Storage & transport | 10% to 90% relative humidity without |
| humidity | condensation |
| Atmospheric pressure | 700 hPa to 1060 hPa |
| Dimension | |
| Physical weight | 76 grams (approximate - including battery) |
| Emission compliance | CISPR 11, Group 1, Class B (Residential |
| | environment) |
| Immunity test level | 10 V/m |
| Type of protection | Internally powered class II ME equipment |
| Degree of protection | Type BF |
| Mode of operation | Continuous |
| Enclosure Degree of | IP55 |
| protection | |
| Biocompatibility | Device is biocompatible per ISO 10993-1 |
| Applied part | Chest electrodes, Finger electrodes including |
| | rest slots, On/OFF button, PCG and diaphragm |

The user should clean and disinfect the electrodes, diaphragm and the body of the device between use, failure to do so may cause infections or allergies to patients. The chest piece diaphragm and electrodes should be disinfected after each use with an alcohol wipe followed by cleaning with a lint-free soft clean and dry cloth. To clean the device, use only a lint-free soft clean and dry cloth. Dry the device completely before use. Do not immerse or soak the device in water or any other form of liquid sterilant for cleaning.

To store the device, keep it in a dry location away from any extreme heat. It is advisable to place the device on a soft surface to avoid damage to the device in general and to the chest electrode side. Storage of device in device package may prevent damage. Built-in Li-Polymer battery may be used only for up to 2 years. Hence the device after 2 years of usage can be sent to manufacturer for battery replacement. Device contains Li-Polymer battery and electronic components; hence device should not be disposed as social waste. Device and its accessories need to be disposed according to electrical and electronic waste disposal guidelines of local regulation. Contact local authorities to dispose device, its parts and accessories.

Regarding the application itself, which is loaded on the user device, the HealthyU application connects to HealthyU device via Bluetooth communication to display, record and replay the ECG and PCG data captured by the HealthyU device. HealthyU application has two screens namely steth screen and 7-channel screen. Steth screen display single channel ECG and PCG, whereas 7-channel screen display all 7 ECG waveforms. The patient file from HealthyU app can be stored locally in user's smart device and can be shared to clinician. Data displayed on the app shall not be used for diagnostic purposes without clinician's consultation in some cases. In others, the system may include algorithmic detection of pathologies. No special training or qualification is required to use the application. HealthyU app screen might display personally identifiable user information, ensure app screen is not viewable to public, while using in public places. For security reasons, all data stored in smart device shall be deleted on the uninstallation of application.

Initially the user should download HealthyU app from the App Store on a compatible android user device. The user then opens the HealthyU app and follows the onscreen instructions to register the HealthyU device. During the first usage, HealthyU app seeks minimum permission required to ensure intended app performance. Click Allow to proceed further. Steth screen is the default landing screen. Click the Bluetooth icon on the steth screen to initiate app scanning the nearby HealthyU device. Click the HealthyU device machine id displayed on the screen to complete pairing. For successful pairing ensure that the smart device Bluetooth is ON, the HealthyU device is ON, and the distance between HealthyU device and smart device is within 10 meters. An example of the pairing screen may be found at 1900 of FIG. 19. On successful pairing the Bluetooth icon displays label as paired in blue color, the HealthyU device's firmware version is displayed on the app screen, and the HealthyU device's battery level is displayed on the app screen.

ECG and PCG waveform are displayed in 2 screens, namely steth screen and 7-channel screen, FIGS. 7 and 8 respectively. Icon in the steth screen labeled as 7-channel and icon in 7-channel screen labeled as steth can be used to switch screens. PCG and channel −2 ECG waveform are displayed in steth screen, whereas 7-channel ECG waveforms are displayed in 7-channel screen. Click the record button to start recording the ECG and PCG waveforms. Before initiating recording refer to instruction for use of HealthyU device and follow the positioning guidelines displayed on screen to position the device on the chest as intended. Set the recoding duration using the settings icon available in the screen. Ensure that the smart device's battery level is above 50% before starting the recording.

Once the recording duration is completed, a preview screen appears. In the preview screen recorded ECG and PCG waveforms are displayed. You can either save or discard the recorded waveforms using the save and discard button. Save the recording to an existing patient profile or add a new patient profile, while saving the recorded waveform the body posture and device location should be entered, using the screen seen at FIG. 10. The saved data are compiled in PDF format and stored in the smart device. This file can be printed or shared over email communication, in this particular embodiment. For each recording one PDF file is generated. Recording and saving actions are specific to steth screen and 7-channel screen. Hence, recording and saving need to be done individually in each screen.

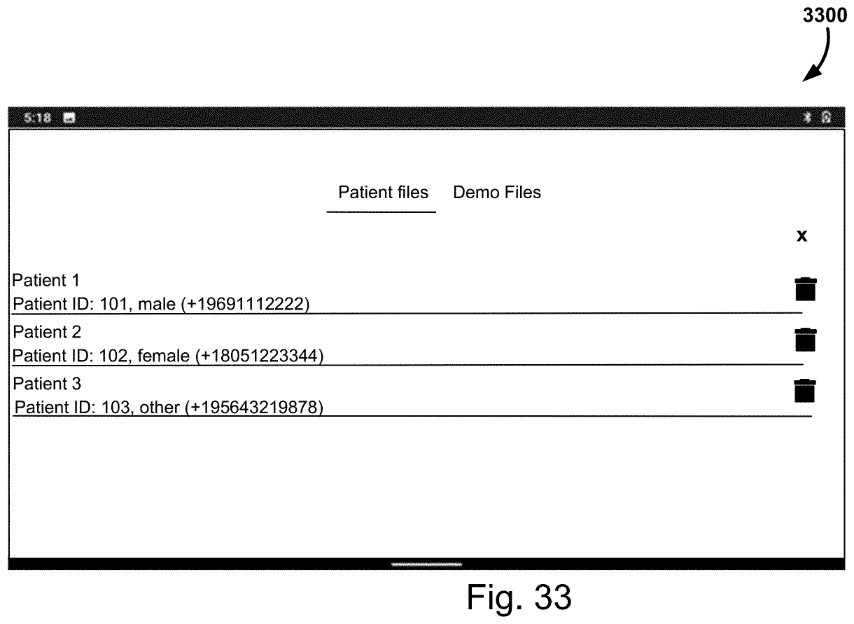

Click on the Open icon (appearing as a file folder) available on the app screen to access the patient files. Patient files are ECG/PCG recordings of users/patients that have been saved in the system. The user can refer to them, whenever needed, based on the following search parameters: Patient ID, Name of patient, and/or Patient/user mobile number. Once a patient match is obtained, based on the above parameters, all the recordings pertaining to that patient would be available. All relevant recordings would be listed along with the timestamp information. User can select the recording of his/her choice and view it in replay mode. While playback is active, all other buttons in the user interface shall be inactive. An example of a screen for accessing patient files can be found at 3300 of FIG. 33.

All recordings are stored in smart device. Recordings listed are searchable using user name, id or contact details. Individual recordings can be selected to view or share. Recordings can either be printed or shared over email. Patient file also has heart rate value indicated corresponding to each recording. Accuracy of heart rate measurement is ±5 beats per minute. Recordings that are stored in smart device are encrypted to ensure data privacy and security. However, data is not secured over email communication.

To access settings the user clicks on setting icon in either steth screen or 7-channel screen. User configurable settings are available in settings screen. As previously discussed, FIGS. 11-13 provide screenshots of the settings screens. Setting include the following options: Select Color—User may be able to change the default colors for the Heart Sound (HS)/PCG and ECG waveform using this option; ECG rhythm Grid—The grid can be enabled for the EKG rhythm, using this option; Edit Patient's Profile—User can use this option to make changes to the current profile of a patient. Patient/user profile includes name, date of birth, gender, contact details and medical history; Edit Doctor's Profile—Clinician can use this option to make changes to the current profile. Clinician profile includes name, expertise, experience, registration id and location; Recording Duration—This option can help the user change the auscultation recording duration from the default of 10 seconds to 20 seconds or 30 seconds etc. to 120 seconds; HealthyU Device registration (email, location, registration code)—First time users are expected to register themselves with HD Medical, Inc. for patient database and customer service purpose; Default auto pairing of selected smart device—For simple and fast connection between user's smart device and HealthyU device, user can enable this option. Every time the smart device detects the previously paired and auto pairing enables HealthyU device, it may automatically get connected; Renaming ECG channel labels—Users as per clinician's recommendation have the option to modify the ECG channel names. This may be done for easy ECG reading based on their standard protocol. The following Table 3 provides an overview of all functions in the application:

TABLE 3
Icons and Functions

| | |
|---|---|
| Icons | Functionality |

Steth Screen and 7-channel screen

| | |
|---|---|
| Record | Initiate recording ECG and PCG waveform from the HealthyU device. |
| | Steth screen and 7-channel screen has dedicated record buttons. |
| Freeze | To screen capture the data present in active screen |
| Add Study | HealthyU app can be used by more than one user by creating |
| | individual user profile. Using Add Study button, user and the |
| | recording can be mapped one to one. |
| Exit Study | Exit study option can be used to switch between user profiles. Select |
| | the profile corresponding to current user. |
| Bluetooth | To pair HealthyU device with app |
| Steth | To view steth screen displaying PCG and Channel - 2 ECG waveform |
| 7-Channel | To view 7-channel ECG waveform |
| Open | To view demo files and Patient file |
| Setting | To view the user configurable settings |
| Device Spec | To view the user's smart device specifications |

Preview Screen

| | |
|---|---|
| Save | To save the recorded waveforms |
| Discard | To discard the recorded waveforms |
| Previous | Recorded waveforms are shown in multiple screens. Previous option |
| | can be used to shift between the multiple screens |
| Next | Recorded waveforms are shown in multiple screens. Next option can |
| | be used to shift between the multiple screens |
| Edit | Body posture and HealthyU device position can be edited |
| Exit | Active session can be exited |

Settings Screen

| | |
|---|---|
| Graph Line | To change ECG waveform color |
| Graph Background | To change ECG waveform background color |
| Graph Label | To change ECG waveform label. Labels can be changed after |
| | consultation with clinician. |
| ECG Grid | To remove or keep ECG graph in the background |
| Gain | It is used to adjust waveform display according to your smart device's |
| | screen size and resolution. |
| Screen Length | It is used to vary the number of ECG and PCG cycles displayed per |
| | screen. |
| Edit Patient's Profile | Add user information such as name, id, gender, age, contact details |
| | and medical history. This information is mapped with ECG and PCG |
| | recordings to enable clinician in diagnosis. |
| Edit Doctor's Profile | Add consulting doctor name, registered id, area of expertise and |
| | experience |
| Disable Auto Pairing | By default auto pairing is enabled. App is capable of connecting |
| | automatically with your HealthyU device based on the previous |
| | pairing. |
| 10 MPA | |
| Recording Time | Recording duration can be changed in multiples of 10 seconds. |
| | Recoding duration range is 10 seconds to 120 seconds. |

Upon completion of each recording, the user may switch off the smart user device's Bluetooth connection, and close all patient files and the application window. The smart user device should be screened for virus attacks at least once in a month to ensure that the data stored in the smart device is not compromised. Use a recognized anti-virus scanning app from App store. The application is updated for security and performance reasons from HD Medical, Inc. team. For such updates, a pop up message may be displayed on the screen to seek permission. In such cases, please accept and install the update.

Some specifications for the medical device 160 are captured in Table 4 below:

TABLE 4
Design Requirements

| | | |
|---|---|---|
| PRD ID | Specification | Rationale/Reference |

Functional Requirements

| | | |
|---|---|---|
| PR0001 | Weighing less than 100 grams | Easy for patient to handle the |
| | | device |
| PR0002 | Degree of protection against harmful | For safe operation in the home |
| | ingress of water and particulate matter: | healthcare environment |
| | IP55 or higher | IEC 60601-1, Clause 6.3 |
| | | Protection against harmful |
| | | ingress of water or particulate |
| | | matter |
| | | Method of testing per IEC |
| | | 60601-2-47, 201.11.6.5 Ingress |
| | | of water or particulate matter |
| | | into ME equipment & ME |
| | | systems, IEC 60601-1-11, |
| | | 8.3.1. |
| PR0003 | Battery life - minimum 5 hours of | Typical use is four 15 min |
| | continuous operation between recharges | sessions per day on a single |
| | | charge. |
| PR0004 | Rechargeable Lithium Polymer Battery; | Provide power to HealthyU |
| | nominal voltage 3.7 V | device via field-replaceable |
| | | and rechargeable battery |
| PR0005 | USB type-C receptacle for connecting | To charge the Healthy U |
| | 5 V battery charger | battery |
| PR0006 | Operating input voltage of charger is 100- | To use in U.S. and EU Market |
| | 240 V | |
| PR0007 | Color LCD Display | Durable |
| | Display Format: 135 × 240 pixel | Easily readable in wide |
| | (RGB) | lighting condition (100- |
| | Dimensions (H × W): 17 mm × 30 mm | 1500 lx) |
| | minimum | Time Display |
| | Active Area (H × W): 14 × 24 mm | Low battery status |
| | minimum | indication |
| | Interface mode: SPI | |
| | Colors: 65K | |
| PR0008 | Bluetooth communication | HealthyU to communicate with |
| | BLE V 5.0 | the application software |
| PR0009 | Accuracy of signal reproduction | Accuracy of signal |
| | | reproduction, input signal in |
| | | range ±5 mv varying at rate |
| | | 125 mV/s shall be reproduced |
| | | with error ≤ ±20% of nominal |
| | | value of the output or ±100 |
| | | μV, whichever is greater. |
| PR0010 | ECG electrodes | Allow device to obtain 7 |
| | ADS1293 module is used for ECG | channel ECG signals from 3 |
| | 3 circular electrodes on chest side | chest electrodes and 2 finger |
| | (back) of device | electrodes |
| | 2 finger electrodes on display side | |
| | (front) of device | |
| PR0011 | PCG waveform of heart sounds | Provide plot of high-fidelity |
| | | recording |
| PR0012 | Application software detects all | Provide connectivity between |
| | HealthyU devices within range of | application software and |
| | Bluetooth connectivity | HealthyU device |
| PR0013 | HealthyU devices listed by name & | Provide option for user to |
| | associated MAC ID in application | select their HealthyU device |
| | software | |
| PR0014 | Audio jack 3.5 mm in HealthyU device to | To allow user to directly listen |
| | connect with user device such as laptop, | to the PCG audio signal in real |
| | tablet to hear PCG audio signal | time |
| PR0015 | Type BF applied part | IEC 60601-1, 8.3 |
| | | Classification of applied parts, |
| | | type BF applied part protection |
| | | since device is intended to |
| | | receive electrophysiological |
| | | signals from patients |
| PR0016 | No energy reduction of Defibrillation | IEC 60601-1, 8.5.5.2 Energy |
| | | reduction |
| PR0017 | Second microphone sensor is available to | |
| | sense and to transfer the sound signals | |
| | captured in the device ambience to | |
| | HealthyU application software | |

Firmware Requirements

| | | |
|---|---|---|
| PR0101 | Communication between HealthyU | Display and save ECG and |
| | device and application software | PCG real time waveform in |
| | | devices like mobile, computer |
| | | or tablet. The saved ECG & |
| | | PCG shall be printed and can |
| | | be accessed by registered |
| | | practitioner. |
| PR0102 | Provide display functionality on | Display real time information |
| | HealthyU device LCD | such as time |
| | | IEC 60417 |
| | | Battery status indication, low |
| | | battery icon blinks |
| | | While USB is connected for |
| | | charging, charging indication |
| | | is displayed both in OFF |
| | | screen & ON screen |
| PR0103 | Device button functionality | Buttons to control the |
| | Power ON/OFF button to switch on/off | HealthyU device |
| | the HealthyU device | |
| PR0104 | Encryption protocol | AES-128 protocol |
| PR0105 | ECG signal is represented by 16 bits and | To increase signal fidelity |
| | PCG signal is represented by 8 bits | |
| PR0106 | Provide power from charged battery | Device is powered via 3.7 V |
| | | rechargeable Lithium battery |
| PR0107 | Charge battery via USB-connected | Battery is recharged via USB- |
| | charger | connected charger |
| PR0108 | Indicate correct battery status on device | Allow user to be aware of |
| | display | battery life between charges |
| PR0109 | Firmware version shall be displayed on | Allow user to confirm the |
| | the device display during start up | firmware version |
| PR0110 | Upgrade/update firmware via JTAG | Allow for device firmware |
| | cable connected to device | updates by manufacturer |
| PR0111 | Device is powered ON when ON/OFF | Power ON/OFF functionality |
| | switch is pressed | |
| PR0112 | Device is powered OFF when ON/OFF | Power ON/OFF functionality |
| | switch is pressed | |
| PR0113 | Ensure device does not turn on when | Proper switch functionality |
| | ON/OFF switch is pressed continuously | |
| PR0114 | Encryption of BLE wireless signals and | AES-128 protocol |
| | data | |
| PR0115 | Incorporate error control processes (bit | Ensure data displayed on |
| | error rate, packet loss, and signal-to- | mobile app accurately |
| | noise ratio) to ensure integrity of | replicates the data obtained by |
| | wireless transmitted data | the device |
| PR0116 | Initialize internal peripherals | Allow microcontroller to |
| | | communicate with external |
| | | modules |
| PR0117 | Watchdog timer to generate system reset | Prevent system lockup if |
| | or processor reset | software is trapped in deadlock |
| PR0118 | Incorporate Universal Asynchronous | Support Bluetooth connectivity |
| | Receive Transmit (UART) channel for | |
| | connection between microcontroller and | |
| | Bluetooth module | |
| PR0119 | Incorporate Smart/Auto Pairing for | Allow user to connect device |
| | connection of device to mobile app | to mobile app in an easy and |
| | | secure fashion |
| PR0120 | Implement correct Bluetooth device | Ensure user connects mobile |
| | name | app to correct HealthyU device |
| PR0121 | Real time data streaming at 115200 bps | Provide real time data |
| | baud rate | streaming with appropriate |
| | | fidelity to HealthyU mobile |
| | | app |
| PR0122 | Include synchronization header byte for | Provide real time data |
| | Bluetooth data transmission | streaming to HealthyU mobile |
| | | app |
| PR0123 | Verify synchronized ECG and PCG data | Ensure synchronized data is |
| | to be transmitted via Bluetooth | transmitted to HealthyU |
| | | mobile app |
| PR0124 | Ensure correct firmware version is | Allow mobile app to display |
| | installed to transmit proper ECG signal | correct ECG signal waveform |
| | to mobile app for display | |
| PR0125 | Reproduce heart sounds at captured | Ensure accurate heart sound |
| | sampling rate | data is captured and |
| | | transmitted to HealthyU |
| | | mobile app |
| PR0126 | Ensure functionality of heart sound/PCG | Heart sounds shall be audible |
| | audio | |
| PR0127 | Ensure HealthyU display is visible under | IEC 60601-1-11 Display shall |
| | various lighting conditions (100-1500 lx) | be visible without glare under |
| | | all lighting conditions |
| PR0128 | Ensure real time ECG & PCG signal data | Real time ECG & PCG signals |
| | is displayed on mobile app when device | shall be displayed on mobile |
| | is in use | app when device is in use |
| PR0129 | At any time one HealthyU device can be | To achieve intended use |
| | paired only with one application. | |
| | Application pairing with device is | |
| | controlled by token based authorization | |
| PR0130 | Encryption protocol AES-128 available | To ensure user privacy and |
| | in the Bluetooth low energy device | information security |
| | encrypts data from device & decrypts the | |
| | same data by application that is defined | |
| | internally in the firmware & software | |
| | algorithms for secure & lossless data | |
| | transfer using Bluetooth technology. | |
| PR0131 | Software safety classification for device | IEC 62304 clause 4.3; |
| | firmware and software is class B related | Guidance for software |
| | to moderate risk for FDA | contained in medical device |
| | | 160s |
| PR0132 | Create software development plan (SDP) | IEC 62304 clause 5.1 |
| | for device firmware | |
| PR0133 | Memory requirement of firmware: | ATSAM3X8E Datasheet |
| | Minimum 128K ROM/256 Bytes RAM | |
| PR0134 | TLV2462 module is used as PCG sensor | To pick up heart sounds and |
| | | convert them to digital signal |

Usability Interface Requirements

| | | |
|---|---|---|
| PR0201 | Power On/OFF switch - Momentary | Allow user to power device on |
| | press to switch ON/OFF of the device | or off |
| PR0202 | Transmit PCG (heart sounds) - provide | Allow user to listen to PCG |
| | “lub” (first) and “dub” (second) sounds | signal |
| | to audio output | |
| PR0203 | Transmit visual display of heart sound | Display of PCG heart sounds |
| | components to application software | on application software |
| PR0204 | Transmit ECG signal to application | Allow user to view ECG signal |
| | software | |
| PR0205 | Battery management - display battery | Determine when battery needs |
| | status on HealthyU device | to be recharged |
| | | Indicate when battery is |
| | | charging |
| | | Indicate when charging is |
| | | completed |
| PR0206 | View display under varied lighting | IEC 60601-1-11; View |
| | conditions | information on display without |
| | | glare under indoor/outdoor |
| | | lighting conditions; including |
| | | bright natural light, darkness, |
| | | and artificial illumination |
| PR0207 | Encrypted secure communications | FDA guidance - Radio |
| | | Frequency Wireless |
| | | Technology in Medical device |
| | | 160s |

Packaging, Storage, and Labeling Requirements

| | | |
|---|---|---|
| PR0301 | Instructions for Use (IFU) | ISO 20417: 2021, IEC 60601-1 |
| | | 7.9 Accompanying documents |
| | | IFU should be available to |
| | | provide guidance and/or be |
| | | used as a reference by the end |
| | | user. |
| PR0302 | Labeling - use of symbols | ISO 15223-1: 2016, IEC 60417, |
| | | IEC 60601-1. Description of |
| | | symbols use to be listed in IFU |
| PR0303 | Expected service life labeling | ISO 20417: 2021 |
| | | ISO 15223-1: 2016 |
| | | Clearly communicate product |
| | | expected service life to end |
| | | user |
| | | Refer PR0502 |
| PR0304 | Storage temperature/Humidity range | Provide information about |
| | | storage conditions during |
| | | transport and intended use |
| | | environment |
| | | IEC 60601-1-11, clause 4.2.2 |
| | | Temperature: −10□ to 40□; |
| | | Relative Humidity: 10% to |
| | | 90% |
| PR0305 | Battery charge time from depletion to | IEC 60601-2-47, 201.7.9.2.101 |
| | 90% charge in normal use is 3 hours | Additional instruction for use, |
| | | b)Internally powered ME |
| | | equipment |
| PR0306 | Packaging | ASTM D4169-16 |
| | | IEC 60601-1-11 clause 4.2.2 |
| | | Level of packaging and |
| | | packaging material required to |
| | | protect and preserve |
| | | functionality of the device |
| | | during supply chain activities |
| PR0307 | Legibility and Durability of Markings on | IEC 60601-1, 7.1.2, 7.1.3. |
| | the device, parts and its accessories | Testing need to be done to |
| | | prove markings may remain |
| | | intact throughout lifetime of |
| | | device |
| PR0308 | On the device labeling to be done for | IEC 60417 |
| | button of the device for user to | |
| | understand its functionality | |
| PR0309 | Mac id to be labeled on the device | For user to verify device name |
| | | and id during smart device |
| | | Bluetooth pairing |

Regulatory and Standard Requirements

| | | |
|---|---|---|
| PR0401 | All materials in contact with patient shall | ISO 10993-1: 2018 |
| | be compatible with the human body for | Minimize patient risk of |
| | up to 24 hours per ISO 10993-1 | reaction caused by the product |
| PR0402 | The materials used shall be phthalate | Phthalates can damage the |
| | free. | liver, kidneys, lungs, and |
| | | reproductive system |
| PR0403 | Device shall be free of Bisphenol A | EU MDR 2017/745 |
| | (BPA) | |
| PR0404 | Device shall be able to withstand | IEC 60601-1-11: 2015 |
| | Mechanical shock, rough handling, etc. | IEC 60601-2-47: 2012 |
| | | IEC 60601-1, 15.3 Mechanical |
| | | Strength, Push test, Drop test, |
| | | molding stress relief test, |
| | | rough handling test. |
| PR0405 | Environmental conditions - operating & | IEC 60601-1-11: 2015 |
| | storage temperature/humidity | IEC 60601-2-47: 2012 |
| PR0406 | Patient leakage current | IEC 60601-1, clause 8 |
| | | IEC 60601-1-11, clause 5 |
| PR0407 | Requirements for safety due to thermal | IEC 60601-1, clause 11.1 - |
| | contact with patient | Protection against excessive |
| | | temperatures and other |
| | | hazards. |
| | | Accessible/Applied parts of |
| | | device made of metals, molded |
| | | materials, plastic, glass, rubber |
| | | should not exceed maximum |
| | | temperature of 48 □ (time t > |
| | | 1 min) & 43 □ (time t > 10 |
| | | min) during normal use |
| PR0408 | Electromagnetic compatibility (EMC) | Device shall meet EMC/EMI |
| | and electromagnetic interference (EMI) | requirements per IEC 60601-1- |
| | | 2: 2020 |
| PR0409 | Transport simulation requirements | ASTM D4169-16 Standard |
| | The packaging and device within shall | Practice for Performance |
| | meet all applicable device | Testing of Shipping Containers |
| | performance/functional requirements | and Systems |
| | following shipping per ASTM D4169-16 | |
| | Standard Practice for Performance | |
| | Testing of Shipping Containers and | |
| | Systems | |
| PR0410 | Cleaning and Disinfection | IEC 60601-1, 7.9.2.12, |
| | Effects of multiple cleanings throughout | Test device robustness to |
| | expected service life of device to be | cleaning for (365 days *2 |
| | evaluated. Cleaning procedure to be | years*4 time a day) 2920 times |
| | given in IFU | |
| PR0411 | Device shall be usable for lay users in | IEC 60601-1-11, IEC 62366 |
| | home healthcare environment, to | validation with lay persons as |
| | enhance safe and effective use of device | intended users |
| | | FDA guidance on Usability |
| PR0412 | Electrodes label and positioning need to | For lay user to understand |
| | be given in IFU | device usage and properly |
| | | capture ECG & PCG |

Device Specification Requirements

| | | |
|---|---|---|
| PR0501 | Expected service life 2 years | Reliability, component life |
| | | expectancy & availability |
| | | studies |
| PR0502 | Distance between HealthyU device & | Refer BL652 Datasheet. |
| | application software need to be within 10 | |
| | meters for reliable data transmission | |

Miscellaneous Requirements

| | | |
|---|---|---|
| PR0601 | The device shall be RoHS and EU | RoHS Directive 2011/65/EU |
| | REACH compliant | REACH Regulation (EC) No. |
| | | 1907/2006 |
| PR0602 | Device shall have curved edges | No sharp edges; reduce risk of |
| | | safety hazard |
| | | IEC 60601-1, clause 9 - |
| | | Protection against Mechanical |
| | | Hazards of ME Equipment and |
| | | ME Systems |

Additionally, firmware product requirements are detailed below in Table 5:

TABLE 5
Software Requirements

| | |
|---|---|
| FRS ID | Requirement |

Healthy U Device Firmware

| | |
|---|---|
| FRS0001 | Software safety classification for device firmware is class B related to |
| | moderate risk |

Functional Requirements

| | |
|---|---|
| FRS0101 | Real-time operating system (RTOS) used for watchdog timer to reset system. |
| | This prevent system lockup if software is trapped in deadlock. |
| FRS0102 | Compiler based on bare-metal C code, which allows to write the ARM |
| | firmware. |
| FRS0103 | Firmware is flashed into ATSAM3X8E ARM microcontroller. |
| FRS0104 | Atmel IDE studio 7 - Integrated Environment Development used to develop |
| | ARM Firmware. |
| FRS0105 | Atmel ICE associated static analysis tool used to debug ARM firmware |
| FRS0106 | Compatibility with Microsoft Visual Studio plug-ins and web browsers |
| | Chrome, Edge, Firefox, Internet Explorer, Opera, Safari |
| FRS0107 | Memory requirement for this firmware to work, Minimum 128K |
| | ROM/256 Bytes RAM |
| FRS0108 | Firmware is to initialize the Interface between Device display and |
| | Microcontroller |
| FRS0109 | When Power ON the HealthyU, LCD displays firmware version number, |
| | name of the device, time & battery status, labels of vitals that allow user to |
| | know firmware version, time and what are all the vital parameters to be |
| | displayed on HealthyU App |
| FRS0110 | Bluetooth of HealthyU device is ON by default, connects with application |
| | software as soon as it receives connection request signal from HealthyU |
| | application software. |
| FRS0111 | To provide real time data streaming with appropriate fidelity to HealthyU |
| | mobile app; Firmware shall meet the baud rate of 115200 bps for Real time |
| | data streaming |
| FRS0112 | Continuous transfer of ECG Signal, Battery Level, Charging Status |
| FRS0113 | Continuous transfer of PCG Signal |
| FRS0114 | To mitigate risks of wireless technology, utilize Bluetooth module built in |
| | security and encryption |
| FRS0115 | HealthyU firmware shall advertise its MAC id to HealthyU app for pairing |
| | with particular device. |
| FRS0116 | 2 KHz Sample rate for PCG Channel 1 & 100 Hz sample rate for microphone |
| | channel 2 |
| | No Filtering |
| FRS0117 | 100 Hz ECG Sample rate for each Channel |
| | No Filtering |
| FRS0118 | Initialize internal peripherals to allow microcontroller to communicate with |
| | external modules. |
| FRS0119 | Analog to Digital conversion (ADC): |
| | The PCG signal from TLV2462 is connected to ADC Channel of the |
| | Microcontroller. The ADC uses the ADC Clock to perform the Analog to |
| | Digital conversions |
| FRS0120 | Serial peripheral Interface (SPI): |
| | The ECG signal from ADS 1293 is connected to microcontroller through |
| | serial data link. |
| FRS0121 | The Real-time Clock (RTC) peripheral is designed for time-of-day clock with |
| | alarm and a two-hundred-year Gregorian calendar, complemented by a |
| | programmable periodic interrupt. The alarm and calendar registers are |
| | accessed by a 32-bit data bus. |
| FRS0122 | Timer Interrupt used to update the global variable for key features such as |
| | ECG and PCG |
| FRS0123 | Data exchange between HealthyU app & device |
| FRS0124 | To ensure user privacy and information security, |
| | Encryption protocol AES-128 available in the Bluetooth low energy device |
| | encrypts data from device & decrypts the same data by application that is |
| | defined internally in the firmware & software algorithms for secure & |
| | lossless data transfer using Bluetooth technology. |
| FRS0125 | Internal software checks & tests using watchdog timer are defined to ensure |
| | device's intended functionality. |

Software System Inputs and Outputs

| | |
|---|---|
| FRS0201 | Data transfer between device and app |
| FRS0202 | Lead 1 (potential between left -LA and right index finger- RA), Lead 2 |
| | (potential between left leg chest electrode -LL and right index finger -RA), |
| | and Vector (potential between V and average of limb electrodes) are sent to |
| | HealthyU application software |
| FRS0203 | Incorporate error control processes (bit error rate, packet loss, and signal-to- |
| | noise ratio) to ensure integrity of wireless transmitted data |
| FRS0204 | Each data packet is generated with sequence number in incremental order to |
| | facilitate integrity of data transmitted to mobile app |
| FRS0205 | Device to transmit data packets at 115200 bps to meet threshold requirements |
| | in mobile app |
| FRS0206 | Scaling of input PCG signal with factor 0 to 2\hat{ }12 |
| FRS0207 | Scaling of input ECG signal with factor 0 to 2\hat{ }24 |

Interfaces with other systems

| | |
|---|---|
| FRS0301 | BLE V 5.0 (and above) communication with mobile application software on |
| | smart devices. |
| FRS0302 | Universal Asynchronous Receive Transmit (UART) channel is incorporated |
| | for connection between microcontroller and Bluetooth module to support |
| | Bluetooth connectivity |
| FRS0303 | Transmit real time data to mobile app at 115200 bps baud rate |
| FRS0304 | Bluetooth data transmissions should contain header byte for synchronization |

Software-driven Information and Operator Messages

| | |
|---|---|
| FRS0401 | Battery Charging Indication |
| | The Amber LED is ON while charging. |
| FRS0402 | Status indicators for Battery Management |
| | Firmware shall provide battery power level using the battery icon: |
| | battery power level: 5 out of 5 bars are lighted. |
| | battery power level: 4 out of 5 bars are lighted. |
| | battery power level: 3 out of 5 bars are lighted. |
| | battery power level: 2 out of 5 bars are lighted. |
| | battery power level: 1 out of 5 bars are lighted. |
| | Low battery level: The LCD Display pop-up indication message (Battery |
| | Low, please charge the device) and the Buzzer is ON from that instant, |
| | device may perform for maybe 30 minutes. This may allow user to know |
| | when to charge. |
| FRS0403 | Firmware shall control status indicators for device function based on the |
| | Realtime Data Signal |

Security Requirements

| | |
|---|---|
| FRS0501 | Firmware shall enable Smart/Auto pairing with mobile app for single point to |
| | point communication. |
| FRS0503 | Secure device from access by unauthorized remote devices; unintended |
| | remote device cannot read the signal from device because of the signal |
| | encryption and packet structure |
| FRS0502 | AES 128 encryption of data transmitted by device through use of BLE 5.0 or |
| | above |

User Interface Requirements

| | |
|---|---|
| FRS0601 | Device enters standby mode when charging battery |
| FRS0602 | Transmit PCG (heart sounds) - provide “lub” (first) and “dub” (second) |
| | sounds to audio output |
| FRS0603 | Transmit visual display of heart sound components to application software |
| FRS0604 | Transmit ECG signal to application software to allow user to view ECG |
| | waveform |
| FRS0605 | Color LCD Display visible under various lighting conditions (100-1500 lx) |
| FRS0606 | Firmware should detect when user presses Power ON/OFF key |
| FRS0607 | |

Data Definition and Database Requirements

| | |
|---|---|
| FRS0701 | Format of packets sending ECG/PCG information. |

Installation and Acceptance Requirements at
Operation and Maintenance Site(s)

| | |
|---|---|
| FRS0801 | BLE Script loading is done via JTAG to support device and mobile app |
| | pairing. |

Operation and Maintenance Requirements

| | |
|---|---|
| FRS0901 | JTAG Cable is used to load the program in HealthyU board. Firmware |
| | loading & upgrading is handled by manufacturer. |
| FRS0902 | JTAG programming, security controlled no read write access. |

User Documentation Requirements

| | |
|---|---|
| FRS1001 | User manual to understand the functionality, handling & maintenance of |
| | device-app interface & user interface |

Regulatory Requirements

| | |
|---|---|
| FRS1201 | Compliance with IEC 62304 |
| FRS1202 | Guidance for the Content of Premarket Submissions for Software Contained |
| | in Medical device 160s- Guidance for Industry and FDA Staff; 2005 |
| FRS1203 | Guidance for Industry and FDA Staff- Radiofrequency wireless technology in |
| | medica devices; 2013 |
| FRS1204 | Guidance for Industry and FDA Staff- off the shelf software use in medical |
| | device 160s; 2019 |
| FRS1205 | Warnings and cautions are provided to user using device labels, package label |
| | and IFU |

Critical requirements/Essential performance

Auto-pairing of device & app
Measurement of ECG & PCG
Maintenance of data integrity & security

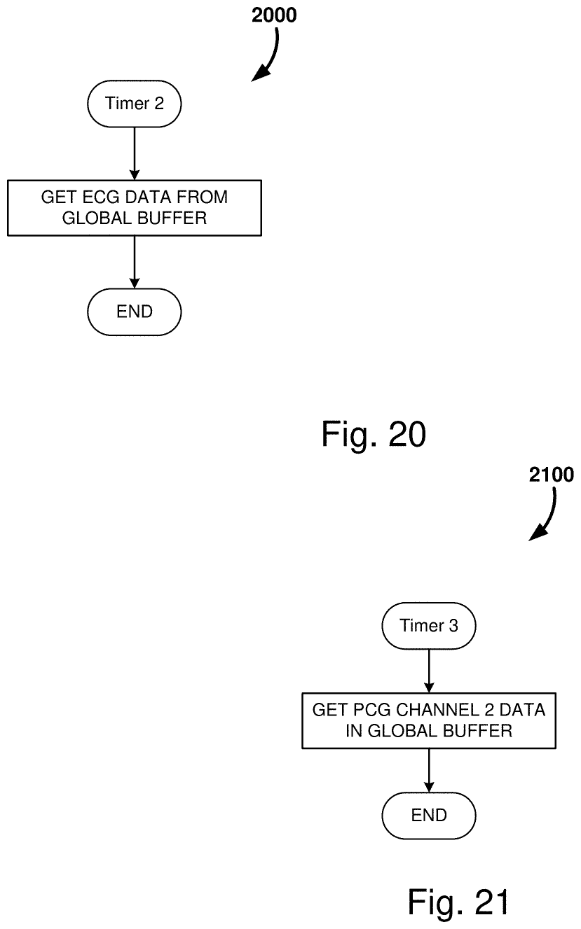

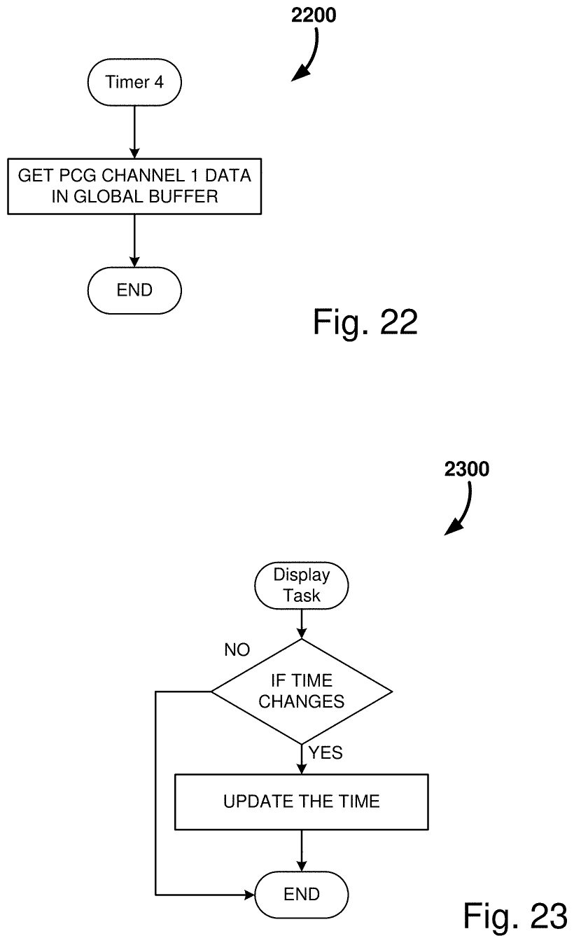

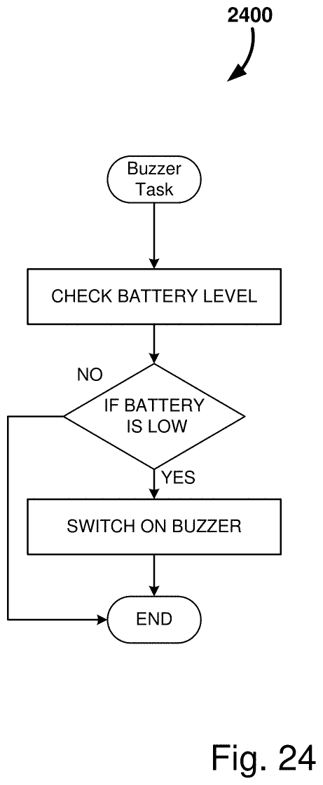

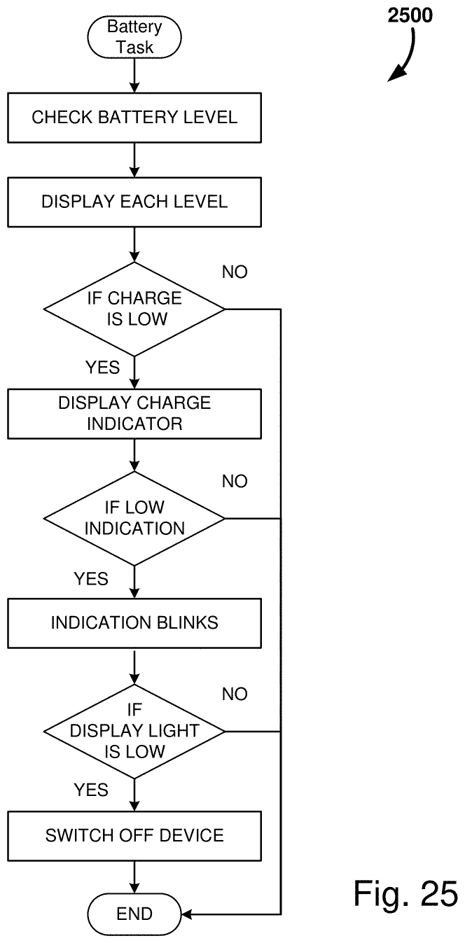

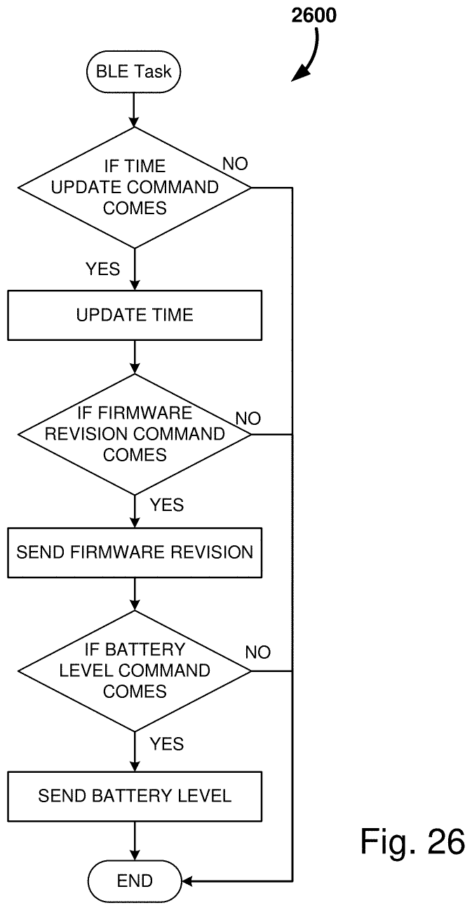

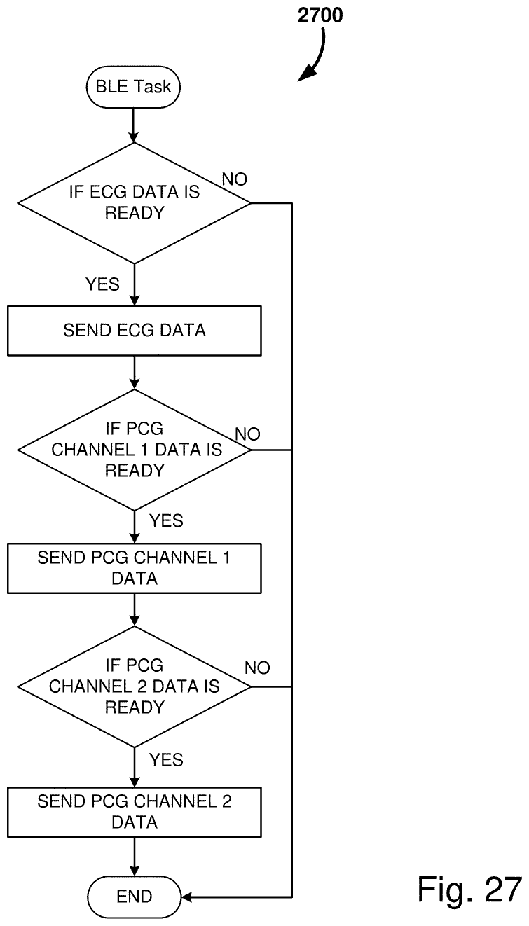

In some substantiations of the medical device 160, multiple timers are employed. When Timer 2 is called the ECG Data is updated to global buffer, as seen at FIG. 20 at 2000. When Timer 3 is called the PCG channel 2 Data is updated to global buffer, as seen at FIG. 21 at 2100. When Timer 4 is called the PCG channel 1 Data is updated to global buffer, as seen at FIG. 22 at 2200. The microcontroller program may check the time when the app is paired with the device. If the time changes, it may update the time on device display, as seen at FIG. 23 at 2300. The microcontroller program may check the battery status, when the battery is low it may switch on buzzer module. FreeRTOS is a real time scheduler (in round-robin manner) for microcontroller program to check the battery status and update the battery icon in display. Battery functioning is seen at FIG. 25 at 2500. FIG. 26, at 2600, defines how the time, firmware revision and battery level data are transferred between app and device. FIG. 27, at 2700, defines how the BLE is transfer ECG and PCG data.

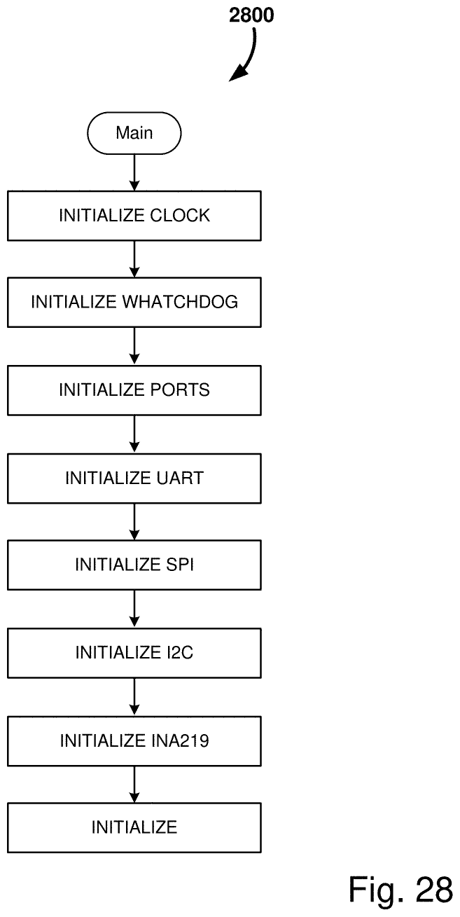

In the medical device 160, in this particular implementation, the microcontroller is used to monitor, process, and interact with the internal as well as external peripherals. When the system is powered on, it may initialize the ports, pins and interfaces of the microcontroller, as seen at FIG. 28 at 2800.

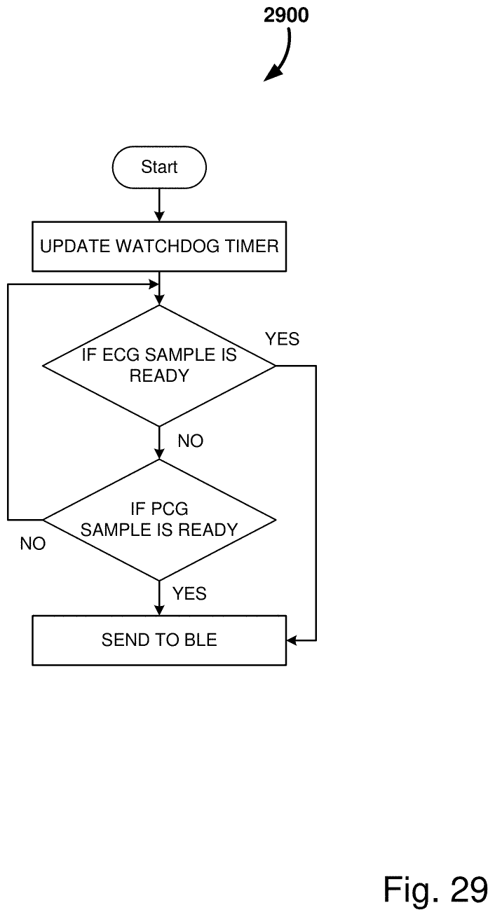

The Watchdog Timer can be used to prevent system lock-up if the software becomes trapped in a deadlock. It features a 12-bit down counter that allows a watchdog period of up to 16 seconds (slow clock at 32 kHz). It can generate a general reset or a processor reset only. In addition, it can be stopped while the processor is in debug mode or idle mode. The reset time of watchdog timer is 1 sec. It may continuously monitor the microcontroller program, to update the ECG or PCG data as per data protocol format and sends to Bluetooth low energy when it is formatted. FIG. 29 illustrates the activity of the watchdog timer, at 2900.

An analog front end (AFE) connects with the microcontroller over SPI channel. Sampling rate of the analog front end is 100 Hz. The ECG signal from AFE is connected via a synchronous serial data link (Serial Peripheral Interface) that provides communication with Analog front end in Master or Slave Mode with Microcontroller for Biopotential. Master Out Slave In (MOSI): This data line supplies the output data from the master microcontroller shifted into the input(s) of the slave analog front end. Master In Slave Out (MISO): This data line supplies the output data from a slave analog front end to the input of the master microcontroller. There may be no more than one slave transmitting data during any particular transfer. Serial Clock (SPCK): This control line is driven by the master and regulates the flow of the data bits. The master may transmit data at a variety of baud rates; the SPCK line cycles once for each bit that is transmitted. Slave Select (NSS): This control line allows slave to be turned on and off by hardware.

The PCG signal from the AFE is connected to ADC Channel of the Microcontroller. The ADC is based on a 12-bit Analog-to-Digital Converter (ADC) managed by an ADC Controller. It also integrates a 16-to-1 analog multiplexer, making possible the analog-to-digital conversions of 16 analog lines. The conversions extend from 0V to ADVREF. The ADC supports an 10-bit or 12-bit resolution mode, and conversion results are reported in a common register for all channels, as well as in a channel-dedicated register. Software trigger, external trigger on rising edge of the ADTRG pin or internal triggers from Timer Counter output(s) are configurable.

The comparison circuitry allows automatic detection of values below a threshold, higher than a threshold, in a given range or outside the range, thresholds and ranges being fully configurable. The ADC Controller internal fault output is directly connected to PWM Fault input. This input can be asserted by means of comparison circuitry in order to immediately put the PWM outputs in a safe state (pure combinational path). The ADC also integrates a Sleep Mode and a conversion sequencer and connects with a PDC channel. These features reduce both power consumption and processor intervention. This ADC has a selectable single-ended or fully differential input and benefits from a 2-bit programmable gain. A whole set of reference voltages is generated internally from a single external reference voltage node that may be equal to the analog supply voltage. An external decoupling capacitance is required for noise filtering. A digital error correction circuit based on the multi-bit redundant signed digit (RSD) algorithm is employed in order to reduce INL and DNL errors.

The ADC uses the ADC Clock to perform the conversions. Converting a single analog value to a 12-bit digital data requires Tracking Clock cycles as defined in the field TRACKTIM of the “ADC MODE REG” and Transfer Clock cycles as defined in the field TRANSFER of the same register. The ADC Clock frequency is selected in the PRESCAL field of the Mode Register (ADC_MR). The tracking phase starts during the conversion of the previous channel. If the tracking time is longer than the conversion time, the tracking phase is extended to the end of the previous conversion. The ADC clock range is between MCK/2, if PRESCAL is 0, and MCK/512, if PRESCAL is set to 255 (0xFF). PRESCAL should be programmed in order to provide an ADC clock frequency. For HealthyU the MCLK is equals to 84 MHz, The ADC clock range is MCK/512 and PRESCAL is set to 255 (0xFF). So the ADC clock frequency is 1.64 KHz.

The conversion is performed on a full range between 0V and the reference voltage pin ADVREF. Analog inputs between these voltages convert to values based on a linear conversion. The ADC supports 10-bit or 12-bit resolutions. The 10-bit selection is performed by setting the LOWRES bit in the ADC Mode Register (ADC_MR). By default, after a reset, the resolution is the highest and the DATA field in the data registers is fully used. By setting the LOWRES bit, the ADC switches to the lowest resolution and the conversion results can be read in the lowest significant bits of the data registers. The two highest bits of the DATA field in the corresponding ADC_CDR register and of the LDATA field in the ADC_LCDR register read 0. Moreover, when a PDC channel is connected to the ADC, 12-bit or 10-bit resolution sets the transfer request size to 16 bits. For HealthyU 12 bit resolution is set by setting LOWRES (4) bit to zero in ADC Mode Register.

When a conversion is completed, the resulting 12-bit digital value is stored in the Channel Data Register (ADC_CDRx) of the current channel and in the ADC Last Converted Data Register (ADC_LCDR). By setting the TAG option in the ADC_EMR, the ADC_LCDR presents the channel number associated to the last converted data in the CHNB field. The channel EOC bit in the Status Register (ADC_SR) is set and the DRDY is set. In the case of a connected PDC channel, DRDY rising triggers a data transfer request. In any case, either EOC and DRDY can trigger an interrupt. Reading one of the ADC_CDR registers clears the corresponding EOC bit. Reading ADC_LCDR clears the DRDY bit and EOC bit corresponding to the last converted channel.

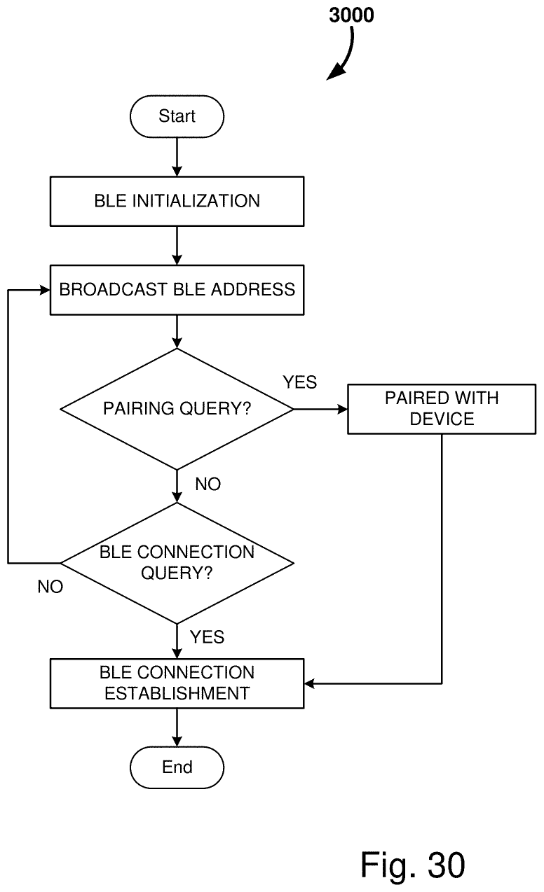

The communication between HealthyU App & HealthyU device is achieved by Bluetooth module. It is a v5.0 single mode Bluetooth module. By default BLE5.0 is aes-128 encrypted. When the device is powered ON and the BLE initialized and transmit its broadcast address, if it receives pairing query from HealthyU App it may acknowledge the pairing query and based on this the connection is established. FIG. 30, at 3000, illustrates this pairing process. BLE module is connect with the microcontroller over UART channel. It provides access to the BLE module 2-wire UART interface (TX, RX).

HealthyU application may sync all HealthyU device as soon as connected over Bluetooth follow by auscultation page. Table 6 provides the command tor time updates:

TABLE 6
Command for Time Update

| | | |
|---|---|---|
| BYTE0 | START_BYTE1 | FE |
| BYTE1 | START_BYTE2 | EF |
| BYTE2 | FrameCount | 1 |
| BYTE3 | RxCommand_Byte | FA |
| BYTE4 | RxLenOfByte | 7 |
| BYTE5 | Rx_Hours | |
| BYTE6 | Rx_Min | |
| BYTE7 | Rx_Sec | |
| BYTE8 | Rx_Day | |
| BYTE9 | Rx_Month | |
| BYTE10 | Rx_YearMSB | |
| BYTE11 | Rx_YearLSB | |
| BYTE12 | Rx_CRC | xx |
| BYTE13 | END Byte | 0 |
| BYTE14 | END Byte | A5 |

Where:

Start of Frame: It is start byte to define start of packet.

FE=START_BYTE1

EF=START_BYTE2

Data Type: 1=Defines the type of the Data.

Frame Count: 1=It define frame count of the packet.

Command Byte: FA=Defines Receiver command byte of Timer update.

Hours: Defines Hours byte

Min: Defines minutes byte

Sec: Defines seconds byte

Date: Defines date byte

Month: Defines month byte

Year MSB: Defines most significant byte of the year

Year LSB: Defines least significant byte of the year

LenOfByte: 7=Defines the Length of the Byte excluding StartByte and EndByte

Rx_CRC: xx=Defines Receiver Cyclic Redundancy check for error detecting.

END Byte: Defines end of packet

0=End byte 1

A5=End byte 2

Table 7 provides ECG records request for the medical device 160 to the application running on the user device:

TABLE 7
ECG Record Request

| | | |
|---|---|---|
| Byte0 | START1 | 0xFE |
| Byte1 | START2 | 0xFB |
| Byte2 | ECG Data type | 0x01 |
| Byte3 | CRC of byte 4 to 15 | |
| Byte4 | ECG_CH1_Value1 MSB | xx |
| Byte5 | ECG_CH1_Value1 LSB | xx |
| Byte6 | ECG_CH1_Value2 MSB | xx |
| Byte7 | ECG_CH1_Value2 LSB | xx |
| Byte8 | ECG_CH2_Value1 MSB | xx |
| Byte9 | ECG_CH2_Value1 LSB | xx |
| Byte10 | ECG_CH2_Value2 MSB | xx |
| Byte11 | ECG_CH2_Value2 LSB | xx |
| Byte12 | ECG_CH3_Value1 MSB | xx |
| Byte13 | ECG_CH3_Value1 LSB | xx |
| Byte14 | ECG_CH3_Value2 MSB | xx |
| Byte15 | ECG_CH3_Value2 LSB | xx |

| | |
|---|---|
| Byte16 | 0 |
| Byte17 | 0 |
| Byte18 | 0 |

| | | |
|---|---|---|
| Byte19 | END Byte | 0 |

Where:

Start of Frame: It is start byte to define start of packet.

0xFE=Byte 0

0xFB=Byte 1

CMD Byte: Defines the Command Byte.

0x01=Byte 2

CRC: Defines CRC for Byte 4 to Byte 15

ECG Data (MSB): xx=Defines Most Significant Byte of the ECG data.

ECG Data (LSB): xx=Defines Least Significant Byte of the ECG Data.

Table 8 provides the PCG record request for the medical device 160 to the application in the user device:

TABLE 8
PCG Record Request

| Byte1 | START1 | 0xFE |
|---|---|---|
| Byte1 | START2 | 0xFB |
| Byte2 | CMD Byte | 0x02 |
| Byte3 | CRC for Byte 4 to 18 | |
| Byte4 | PCG Data 1 | xx |
| Byte5 | PCG Data 2 | xx |
| Byte6 | PCG Data 3 | xx |
| Byte7 | PCG Data 4 | xx |
| Byte8 | PCG Data 5 | xx |
| Byte9 | PCG Data 6 | xx |
| Byte10 | PCG Data 7 | xx |
| Byte11 | PCG Data 8 | xx |
| Byte12 | PCG Data 9 | xx |
| Byte13 | PCG Data 10 | xx |
| Byte14 | PCG Data 11 | xx |
| Byte15 | PCG Data 12 | xx |
| Byte16 | PCG Data 13 | xx |
| Byte17 | PCG Data 14 | xx |
| Byte18 | PCG Data 15 | xx |
| Byte19 | END Byte | 0 |

Where:

Start of Frame: It is start byte to define start of packet.

0xFE=Byte 1

0xEF=Byte 2

CMD Byte: Defines the Command Byte.

0x02=Byte 2

CRC: Defines CRC for Byte 4 to Byte 18

PCG Data: xx=Defines the PCG data.

Table 9 provides a second sensor record request for the medical device 160 from the application on the user device:

TABLE 9
Second Sensor Record Request

| Byte1 | START1 | 0xFE |
|---|---|---|
| Byte1 | START2 | 0xFB |
| Byte2 | CMD Byte | 0x03 |
| Byte3 | CRC for Byte 4 to 18 | |
| Byte4 | Second sensor Data 1 | xx |
| Byte5 | Second sensor Data 2 | xx |
| Byte6 | Second sensor Data 3 | xx |
| Byte7 | Second sensor Data 4 | xx |
| Byte8 | Second sensor Data 5 | xx |
| Byte9 | Second sensor Data 6 | xx |
| Byte10 | Second sensor Data 7 | xx |
| Byte11 | Second sensor Data 8 | xx |
| Byte12 | Second sensor Data 9 | xx |
| Byte13 | Second sensor Data 10 | xx |
| Byte14 | Second sensor Data 11 | xx |
| Byte15 | Second sensor Data 12 | xx |
| Byte16 | Second sensor Data 13 | xx |
| Byte17 | Second sensor Data 14 | xx |
| Byte18 | Second sensor Data 15 | xx |
| Byte19 | END Byte | 0 |

Where:

Start of Frame: It is start byte to define start of packet.

0xFE=Byte 1

0xEF=Byte 2

CMD Byte: Defines the Command Byte.

0x02=Byte 2

CRC: Defines CRC for Byte 4 to Byte 18

PCG Data: xx=Defines the PCG data

Table 10 provides a device battery level request:

TABLE 10
Device Battery Level Request

| | | |
|---|---|---|
| Byte1 | START1 | 0xFE |
| Byte2 | START2 | 0xEF |
| Byte3 | Cmd Byte | 0x64 |
| Byte4 | Len of Data | 3 |
| Byte5 | BATLevel_MSB | |
| Byte6 | BATLevel_LSB | |
| Byte7 | Charge Status | 1 |
| Byte8 | CRC of Data | |
| Byte9 | END1 | 0 |
| Byte10 | END2 | 0xA5 |

Where:

Start of Frame: It is start byte to define start of packet.

0xFE=Byte 1

0xEF=Byte 2

Command Byte: 0x64=Defines the Command Byte.

Length of data: 3=It define complete length of Data.

BATLevel MSB: Defines Most Significant Byte of the battery level data.

BATLevel LSB: Defines Least Significant Byte of the battery level data.

Charge Status: 1=Defines charge status of the device

CRC of Data: Defines the Cyclic Redundancy Check of data for error detecting

End of Frame: Defines End of frame

0=End Byte 1

0xA5=End Byte 2

Table 11 provides the application to medical device 160 version command:

TABLE 11
Device Version Command

| | | |
|---|---|---|
| BYTE0 | START_BYTE1 | FE |
| BYTE1 | START_BYTE2 | EF |
| BYTE2 | FrameCount | 1 |
| BYTE3 | RxCommand_Byte | 0xFB |
| BYTE4 | RxLenOfByte | 0 |
| BYTE5 | Feature Use | 0 |
| BYTE6 | Feature Use | 0 |
| BYTE7 | CRC of Data | |
| BYTE8 | END1 | 0 |
| BYTE9 | END2 | 0xA5 |

Where:

Start of Frame: It is start byte to define start of packet.
FE=Byte 1
EF=Byte 2

Frame Count: 1=Defines Frame Count

Command Byte: 0xFB=Defines the Command Byte.

Length of data: 0=It define complete length of Data.

Feature Use: 0=Defines Feature Use of Device version.

Feature Use: 0=Defines Feature Use of Device version.

CRC of Data: Defines the Cyclic Redundancy Check of data for error detecting

End of Frame: Defines End of frame

0=End Byte 1

0xA5=End Byte 2

Table 12 provides the medical device 160 to the application version command:

TABLE 12
Application Version Command

| | | |
|---|---|---|
| Byte1 | START1 | 0xFE |
| Byte2 | START2 | 0xEF |
| Byte3 | Cmd Byte | 0x65 |
| Byte4 | CRC of data | 4 |
| Byte5 | Firmware Version Byte1 | |
| Byte6 | Firmware Version Byte2 | |
| Byte7 | Firmware Version Byte3 | 1 |
| Byte8 | Firmware Version Byte4 | |
| Byte9 | END1 | 0 |
| Byte10 | END2 | 0xA5 |

Where:

Start of Frame: It is start byte to define start of packet.

0xFE=Byte 1

0xEF=Byte 2

Command Byte: 0x65=HealthyU device to HealthyU App command Byte

CRC: 4=It define the CRC of data.

Firmware Version Byte1: Defines Firmware Version

Firmware Version Byte2: Defines Firmware Version

Firmware Version Byte3: 1=Defines Firmware Version

Firmware Version Byte4: Defines Firmware Version

End of Frame: Defines End of frame

0=End Byte 1

0×A5=End Byte 2

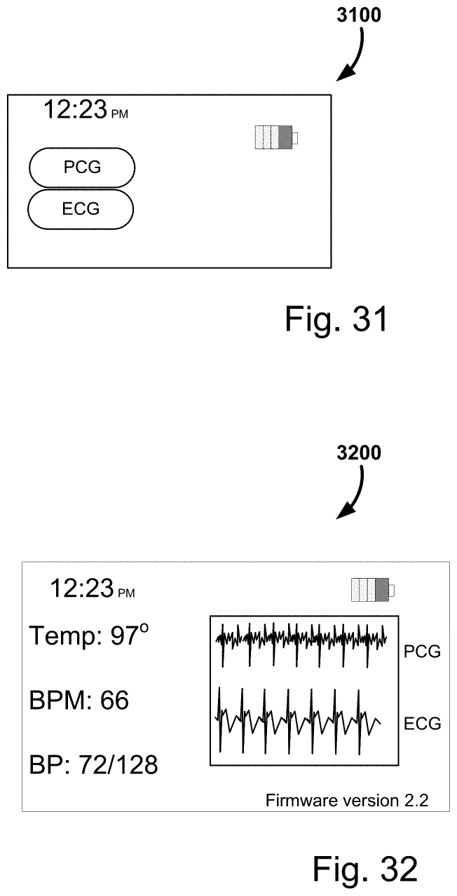

An LCD is used for displaying Time, PCG icon with Text, ECG icon with Text and Battery status icon in default. The supply voltage for the LCD is LCD_3V3D. FIGS. 31 and 32 provide two different screen renderings. In FIG. 31, the time, battery indicator, and sensory icons are illustrated, at 3100. In an alternate display, seen at FIG. 32, the pulse rate, heart rate, battery, time, blood pressure, and ECG/PCG signals may also be displayed, at 3200. Table 13 provides a set of example LCD system commands:

TABLE 13
LCD System Commands

| Instruction | D/CX | WRX | RDX | D17-8 | D7 | D6 | D5 | D4 | D3 | D2 | D1 | D0 | Hex | Function |
|---|---|---|---|---|---|---|---|---|---|---|---|---|---|---|
| NOP | 0 | ↑ | 1 | — | 0 | 0 | 0 | 0 | 0 | 0 | 0 | 0 | (00h) | No |
| | | | | | | | | | | | | | | operation |
| SWRESET | 0 | ↑ | 1 | — | 0 | 0 | 0 | 0 | 0 | 0 | 0 | 1 | (01h) | Software |
| | | | | | | | | | | | | | | reset |
| RDDID | 0 | ↑ | 1 | — | 0 | 0 | 0 | 0 | 0 | 1 | 0 | 0 | (04h) | Read display |
| | | | | | | | | | | | | | | ID |
| | 1 | 1 | ↑ | — | — | — | — | — | — | — | — | — | | Dummy read |
| | 1 | 1 | ↑ | — | ID17 | ID16 | ID15 | ID14 | ID13 | ID12 | ID11 | ID10 | | ID1 read |
| | 1 | 1 | ↑ | — | ID27 | ID26 | ID25 | ID24 | ID23 | ID22 | ID21 | ID20 | | ID2 read |
| | 1 | 1 | ↑ | — | ID37 | ID36 | ID35 | ID34 | ID33 | ID32 | ID31 | ID30 | | ID3 read |
| RDDST | 0 | ↑ | 1 | — | 0 | 0 | 0 | 0 | 1 | 0 | 0 | 1 | (09h) | Read display |
| | | | | | | | | | | | | | | status |
| | 1 | 1 | ↑ | — | — | — | — | — | — | — | — | — | | Dummy read |
| | 1 | 1 | ↑ | — | BSTON | MY | MX | MV | ML | RGB | MH | ST24 | | — |
| | 1 | 1 | ↑ | — | ST23 | IFPF2 | IFPF1 | IFPF0 | IDMON | PTLON | SLOUT | NORON | | — |
| | 1 | 1 | ↑ | — | ST15 | ST14 | INVON | ST12 | ST11 | DISON | TEON | GCS2 | | — |
| | 1 | 1 | ↑ | — | GCS1 | GCS0 | TEM | ST4 | ST3 | ST2 | ST1 | ST0 | | — |
| RDDPM | 0 | ↑ | 1 | — | 0 | 0 | 0 | 0 | 1 | 0 | 1 | 0 | (0Ah) | Read display |
| | | | | | | | | | | | | | | power |
| | 1 | 1 | ↑ | — | — | — | — | — | — | — | — | — | | Dummy read |
| | 1 | 1 | ↑ | — | BSTON | IDMON | PTLON | SLPOUT | NORON | DISON | 0 | 0 | | — |
| RDDMADCTL | 0 | ↑ | 1 | — | 0 | 0 | 0 | 0 | 1 | 0 | 1 | 1 | (0Bh) | Read display |
| | 1 | 1 | ↑ | — | — | — | — | — | — | — | — | — | | Dummy read |
| | 1 | 1 | ↑ | — | MY | MX | MV | ML | RGB | MH | 0 | 0 | | — |
| RDDCOLMOD | 0 | ↑ | 1 | — | 0 | 0 | 0 | 0 | 1 | 1 | 0 | 0 | (0Ch) | Read display |
| | | | | | | | | | | | | | | pixel |
| | 1 | 1 | ↑ | — | — | — | — | — | — | — | — | — | | Dummy read |
| | 1 | 1 | ↑ | — | 0 | D6 | D5 | D4 | 0 | D2 | D1 | D0 | | — |
| RDDIM | 0 | ↑ | 1 | — | 0 | 0 | 0 | 0 | 1 | 1 | 0 | 1 | (0Dh) | Read display |
| | | | | | | | | | | | | | | image |
| | 1 | 1 | ↑ | — | — | — | — | — | — | — | — | — | | Dummy read |
| | 1 | 1 | ↑ | — | VSSON | 0 | INVON | 0 | 0 | GC2 | GC1 | GC0 | | — |
| RDDSM | 0 | ↑ | 1 | — | 0 | 0 | 0 | 0 | 1 | 1 | 1 | 0 | (0Eh) | Read display |
| | | | | | | | | | | | | | | signal |
| | 1 | 1 | ↑ | — | — | — | — | — | — | — | — | — | | Dummy read |
| | 1 | 1 | ↑ | — | TEON | TEM | 0 | 0 | 0 | 0 | 0 | 0 | | — |
| RDDSDR | 0 | ↑ | 1 | — | 0 | 0 | 0 | 0 | 1 | 1 | 1 | 1 | (0Fh) | Read display |
| | | | | | | | | | | | | | | self- |
| | | | | | | | | | | | | | | diagnostic |
| | | | | | | | | | | | | | | result |
| | 1 | 1 | ↑ | — | — | — | — | — | — | — | — | — | | Dummy read |
| | 1 | 1 | ↑ | — | D7 | D6 | 0 | 0 | 0 | 0 | 0 | 0 | | — |
| SLPIN | 0 | ↑ | 1 | — | 0 | 0 | 0 | 1 | 0 | 0 | 0 | 0 | (10h) | Sleep in |
| SLPOUT | 0 | ↑ | 1 | — | 0 | 0 | 0 | 1 | 0 | 0 | 0 | 1 | (11h) | Sleep out |
| PTLON | 0 | ↑ | 1 | — | 0 | 0 | 0 | 1 | 0 | 0 | 1 | 0 | (12h) | Partial |
| | | | | | | | | | | | | | | mode on |
| NORON | 0 | ↑ | 1 | — | 0 | 0 | 0 | 1 | 0 | 0 | 1 | 1 | (13h) | Partial off |
| | | | | | | | | | | | | | | (Normal) |
| INVOFF | 0 | ↑ | 1 | — | 0 | 0 | 1 | 0 | 0 | 0 | 0 | 0 | (20h) | Display |
| | | | | | | | | | | | | | | inversion off |
| INVON | 0 | ↑ | 1 | — | 0 | 0 | 1 | 0 | 0 | 0 | 0 | 1 | (21h) | Display |
| | | | | | | | | | | | | | | inversion on |
| GAMSET | 0 | ↑ | 1 | — | 0 | 0 | 1 | 0 | 0 | 0 | 0 | 1 | (26h) | Display |
| | 1 | ↑ | 1 | — | 0 | 0 | 0 | 0 | GC3 | GC2 | GC1 | GC0 | | inversion on |
| DISPOFF | 0 | ↑ | 1 | — | 0 | 0 | 1 | 0 | 1 | 0 | 0 | 0 | (28h) | Display off |
| DISPON | 0 | ↑ | 1 | — | 0 | 0 | 1 | 0 | 1 | 0 | 0 | 1 | (29h) | Display on |
| CASET | 0 | ↑ | 1 | — | 0 | 0 | 1 | 0 | 1 | 0 | 1 | 0 | (2Ah) | Column |
| | | | | | | | | | | | | | | address set |
| | 1 | ↑ | 1 | — | XS15 | XS14 | XS13 | XS12 | XS11 | XS10 | XS9 | XS8 | | X address |
| | 1 | ↑ | 1 | | XS7 | XS6 | XS5 | XS4 | XS3 | XS2 | XS1 | XS0 | | start: 0 ≤ |
| | | | | | | | | | | | | | | XS ≤ X |
| | 1 | ↑ | 1 | | XE15 | XE14 | XE13 | XE12 | XE11 | XE10 | XE9 | XE8 | | X address |
| | 1 | ↑ | 1 | | XE7 | XE6 | XE5 | XE4 | XE3 | XE2 | XE1 | XE0 | | start: S ≤ |
| | | | | | | | | | | | | | | XE ≤ X |
| RASET | 0 | ↑ | 1 | — | 0 | 0 | 1 | 0 | 1 | 0 | 1 | 1 | (2Bh) | Row |
| | | | | | | | | | | | | | | address set |
| | 1 | ↑ | 1 | — | YS15 | YS14 | YS13 | YS12 | YS11 | YS10 | YS9 | YS8 | | Y address |
| | 1 | ↑ | 1 | | YS7 | YS6 | YS5 | YS4 | YS3 | YS2 | YS1 | YS0 | | start: 0 ≤ |
| | | | | | | | | | | | | | | YS ≤ Y |
| | 1 | ↑ | 1 | | YE15 | YE14 | YE13 | YE12 | YE11 | YE10 | YE9 | YE8 | | Y address |
| | 1 | ↑ | 1 | | YE7 | YE6 | YE5 | YE4 | YE3 | YE2 | YE1 | YE0 | | start: S ≤ |
| | | | | | | | | | | | | | | YE ≤ Y |
| RAMWR | 0 | ↑ | 1 | — | 0 | 0 | 1 | 0 | 1 | 1 | 0 | 0 | (2Ch) | Memory write |
| | 1 | ↑ | 1 | D1[17:8] | D1[7] | D1[6] | D1[5] | D1[4] | D1[3] | D1[2] | D1[1] | D1[0] | | Write data |
| | 1 | ↑ | 1 | Dx[17:8] | Dx[7] | Dx[6] | Dx[5] | Dx[4] | Dx[3] | Dx[2] | Dx[1] | Dx[0] | | |
| | 1 | ↑ | 1 | Dn[17:8] | Dn[7] | Dn[6] | Dn[5] | Dn[4] | Dn[3] | Dn[2] | Dn[1] | Dn[0] | | |
| RAMRD | 0 | ↑ | 1 | — | 0 | 0 | 1 | 0 | 1 | 1 | 1 | 0 | (2Eh) | Memory read |
| | 1 | 1 | ↑ | — | — | — | — | — | — | — | — | — | | Dummy read |
| | 1 | 1 | ↑ | D1[17:8] | D1[7] | D1[6] | D1[5] | D1[4] | D1[3] | D1[2] | D1[1] | D1[0] | | Read data |
| | 1 | 1 | ↑ | Dx[17:8] | Dx[7] | Dx[6] | Dx[5] | Dx[4] | Dx[3] | Dx[2] | Dx[1] | Dx[0] | | |
| | 1 | 1 | ↑ | Dn[17:8] | Dn[7] | Dn[6] | Dn[5] | Dn[4] | Dn[3] | Dn[2] | Dn[1] | Dn[0] | | |
| PTLAR | 0 | ↑ | 1 | — | 0 | 0 | 1 | 1 | 0 | 0 | 0 | 0 | (30h) | Partial |
| | | | | | | | | | | | | | | start/end |
| | | | | | | | | | | | | | | address set |
| | 1 | ↑ | 1 | — | PSL15 | PSL14 | PSL13 | PSL12 | PSL11 | PSL10 | PSL9 | PSL8 | | Partial start |
| | | | | | | | | | | | | | | address: |
| | | | | | | | | | | | | | | (0, 1, |
| | | | | | | | | | | | | | | 2, . . . P) |
| | 1 | ↑ | 1 | — | PSL7 | PSL6 | PSL5 | PSL4 | PSL3 | PSL2 | PSL1 | PSL0 | | |
| | 1 | ↑ | 1 | — | PEL15 | PEL14 | PEL13 | PEL12 | PEL11 | PEL10 | PEL9 | PEL8 | | Partial end |
| | | | | | | | | | | | | | | address(0, |
| | | | | | | | | | | | | | | 1, 2, 3,, P) |
| | 1 | ↑ | 1 | — | PEL7 | PEL6 | PEL5 | PEL4 | PEL3 | PEL2 | PEL1 | PEL0 | | |
| VSCRDEF | 0 | ↑ | 1 | — | 0 | 0 | 1 | 1 | 0 | 0 | 1 | 1 | (33h) | Vertical |
| | | | | | | | | | | | | | | scrolling |
| | | | | | | | | | | | | | | definition |
| | 1 | ↑ | 1 | — | TFA15 | TFA14 | TFA13 | TFA12 | TFA11 | TFA10 | TFA9 | TFA8 | | |
| | 1 | ↑ | 1 | — | TFA7 | TFA6 | TFA5 | TFA4 | TFA3 | TFA2 | TFA1 | TFA0 | | |
| | 1 | ↑ | 1 | — | VSA15 | VSA14 | VSA13 | VSA12 | VSA11 | VSA10 | VSA9 | VSA8 | | |
| | 1 | ↑ | 1 | — | VSA7 | VSA6 | VSA5 | VSA4 | VSA3 | VSA2 | VSA1 | VSA0 | | |
| | 1 | ↑ | 1 | — | BFA15 | BFA14 | BFA13 | BFA12 | BFA11 | BFA10 | BFA9 | BFA8 | | |
| | 1 | ↑ | 1 | — | BFA7 | BFA6 | BFA5 | BFA4 | BFA3 | BFA2 | BFA1 | BFA0 | | |
| TEOFF | 0 | ↑ | 1 | — | 0 | 0 | 1 | 1 | 0 | 1 | 0 | 0 | (34h) | Tearing |
| | | | | | | | | | | | | | | effect |
| | | | | | | | | | | | | | | line off |
| TEON | 0 | ↑ | 1 | — | 0 | 0 | 1 | 1 | 0 | 1 | 0 | 1 | (35h) | Tearing |
| | | | | | | | | | | | | | | effect |
| | | | | | | | | | | | | | | line on |
| | 1 | ↑ | 1 | — | — | — | — | — | — | — | — | TEM | | |
| MADCTL | 0 | ↑ | 1 | — | 0 | 0 | 1 | 1 | 0 | 1 | 1 | 0 | (36h) | Memory data |
| | | | | | | | | | | | | | | access |
| | | | | | | | | | | | | | | control |
| | 1 | ↑ | 1 | — | MY | MX | MV | ML | RGB | 0 | 0 | 0 | | — |
| VSCRSADD | 0 | ↑ | 1 | — | 0 | 0 | 1 | 1 | 0 | 1 | 1 | 1 | (37h) | Vertical |
| | | | | | | | | | | | | | | scrolling |
| | | | | | | | | | | | | | | start |
| | | | | | | | | | | | | | | address |
| | 1 | ↑ | 1 | — | VSP15 | VSP14 | VSP13 | VSP12 | VSP11 | VSP10 | VSP9 | VSP8 | | |
| | 1 | ↑ | 1 | — | VSP7 | VSP6 | VSP5 | VSP4 | VSP3 | VSP2 | VSP1 | VSP0 | | |
| IDMOFF | 0 | ↑ | 1 | — | 0 | 0 | 1 | 1 | 1 | 0 | 0 | 0 | (38h) | Idle mode |
| | | | | | | | | | | | | | | off |
| IDMON | 0 | ↑ | 1 | — | 0 | 0 | 1 | 1 | 1 | 0 | 0 | 1 | (39h) | Idle mode |
| | | | | | | | | | | | | | | on |
| COLMOD | 0 | ↑ | 1 | — | 0 | 0 | 1 | 1 | 1 | 0 | 1 | 0 | (3Ah) | Interface |
| | | | | | | | | | | | | | | pixel format |
| | 1 | ↑ | 1 | — | 0 | D6 | D5 | D4 | 0 | D2 | D1 | D0 | | Interface |
| | | | | | | | | | | | | | | format |
| RAMWRC | 0 | ↑ | 1 | — | 0 | 0 | 1 | 1 | 1 | 1 | 0 | 0 | (3Ch) | Memory write |
| | | | | | | | | | | | | | | continue |
| | 1 | ↑ | 1 | D1[17:8] | D1[7] | D1[6] | D1[5] | D1[4] | D1[3] | D1[2] | D1[1] | D1[0] | | Write data |
| | 1 | ↑ | 1 | D1[17:8] | Dx[7] | Dx[6] | Dx[5] | Dx[4] | Dx[3] | Dx[2] | Dx[1] | Dx[0] | | |
| | 1 | ↑ | 1 | D1[17:8] | Dn[7] | Dn[6] | Dn[5] | Dn[4] | Dn[3] | Dn[2] | Dn[1] | Dn[0] | | |
| RAMRDC | 0 | ↑ | 1 | — | 0 | 0 | 1 | 1 | 1 | 1 | 1 | 0 | (3Eh) | Memory read |
| | | | | | | | | | | | | | | continue |
| | 1 | 1 | ↑ | — | — | — | — | — | — | — | — | — | | Dummy Read |
| | 1 | 1 | ↑ | D1[17:8] | D1[7] | D1[6] | D1[5] | D1[4] | D1[3] | D1[2] | D1[1] | D1[0] | | |
| | 1 | 1 | ↑ | Dx[17:8] | Dx[7] | Dx[6] | Dx[5] | Dx[4] | Dx[3] | Dx[2] | Dx[1] | Dx[0] | | |
| | 1 | 1 | ↑ | Dn[17:8] | Dn[7] | Dn[6] | Dn[5] | Dn[4] | Dn[3] | Dn[2] | Dn[1] | Dn[0] | | |
| TESCAN | 0 | ↑ | 1 | — | 0 | 1 | 0 | 0 | 0 | 1 | 0 | 0 | (44h) | Set tear |
| | | | | | | | | | | | | | | scanline |
| | 1 | ↑ | 1 | — | N15 | N14 | N13 | N12 | N11 | N10 | N9 | N8 | | |
| | 1 | ↑ | 1 | — | N7 | N6 | N5 | N4 | N3 | N2 | N1 | N0 | | |
| RDTESCAN | 0 | ↑ | 1 | — | 0 | 1 | 0 | 0 | 0 | 1 | 0 | 1 | (45h) | Get |
| | | | | | | | | | | | | | | scanline |
| | 1 | 1 | ↑ | — | — | — | — | — | — | — | — | — | | Dummy Read |
| | 1 | 1 | ↑ | — | — | — | — | — | — | — | N9 | N8 | | |
| | 1 | 1 | ↑ | — | N7 | N6 | N5 | N4 | N3 | N2 | N1 | N0 | | |
| WRDISBV | 0 | ↑ | 1 | — | 0 | 1 | 0 | 1 | 0 | 0 | 0 | 1 | (51h) | Write display |
| | | | | | | | | | | | | | | brightness |
| | 1 | ↑ | 1 | — | DBV7 | DBV6 | DBV5 | DBV4 | DBV3 | DBV2 | DBV1 | DBV0 | | |
| RDDISBV | 0 | ↑ | 1 | — | 0 | 1 | 0 | 1 | 0 | 0 | 1 | 0 | (52h) | Read display |
| | | | | | | | | | | | | | | brightness |
| | | | | | | | | | | | | | | value |
| | 1 | 1 | ↑ | — | | | | | | | | | | Dummy read |
| | 1 | 1 | ↑ | — | DBV7 | DBV6 | DBV5 | DBV4 | DBV3 | DBV2 | DBV1 | DBV0 | | |
| WRCTRLD | 0 | ↑ | 1 | — | 0 | 1 | 0 | 1 | 0 | 0 | 1 | 1 | (53h) | Write CTRL |
| | | | | | | | | | | | | | | display |
| | 1 | ↑ | 1 | — | 0 | 0 | BCTRL | 0 | DD | BL | 0 | 0 | | |
| RDCTRLD | 0 | ↑ | 1 | — | 0 | 1 | 0 | 1 | 0 | 1 | 0 | 0 | (54h) | Read CTRL |
| | | | | | | | | | | | | | | value display |
| | 1 | 1 | ↑ | — | — | — | — | — | — | — | — | — | | Dummy read |
| | 1 | 1 | ↑ | — | 0 | 0 | BCTRL | 0 | DD | BL | 0 | 0 | | |
| WRCACE | 0 | ↑ | 1 | — | 0 | 1 | 0 | 1 | 0 | 1 | 0 | 1 | (55h) | Write content |
| | | | | | | | | | | | | | | adaptive |
| | | | | | | | | | | | | | | brightness |
| | | | | | | | | | | | | | | control and |
| | | | | | | | | | | | | | | Color |
| | | | | | | | | | | | | | | enhancement |
| | 1 | ↑ | 1 | — | CECTRL | 0 | CE1 | CEO | 0 | 0 | C1 | C0 | | |
| RDCABC | 0 | ↑ | 1 | — | 0 | 1 | 0 | 1 | 0 | 1 | 1 | 0 | (56h) | Read |
| | | | | | | | | | | | | | | content |
| | | | | | | | | | | | | | | adaptive |
| | | | | | | | | | | | | | | brightness |
| | | | | | | | | | | | | | | control |
| | 1 | 1 | ↑ | — | — | — | — | — | — | — | — | — | | Dummy read |
| | 1 | 1 | ↑ | — | 0 | CECTRL | 0 | 0 | 0 | 0 | C1 | C0 | | |
| WRCABCMB | 0 | ↑ | 1 | — | 0 | 1 | 0 | 1 | 1 | 1 | 1 | 0 | (5Eh) | Write CABC |
| | | | | | | | | | | | | | | minimum |
| | | | | | | | | | | | | | | brightness |
| | 1 | ↑ | 1 | — | CMB7 | CMB6 | CMB5 | CMB4 | CMB3 | CMB2 | CMB1 | CMB0 | | |
| RDCABCMB | 0 | ↑ | 1 | — | 0 | 1 | 0 | 1 | 1 | 1 | 1 | 1 | (5Fh) | Read CABC |
| | | | | | | | | | | | | | | minimum |
| | | | | | | | | | | | | | | brightness |
| | 1 | 1 | ↑ | — | — | — | — | — | — | — | — | — | | Dummy read |
| | 1 | 1 | ↑ | — | CMB7 | CMB6 | CMB5 | CMB4 | CMB3 | CMB2 | CMB1 | CMB0 | | |
| | 0 | ↑ | 1 | — | 0 | 1 | 1 | 0 | 1 | 0 | 0 | 0 | (68h) | Read |
| | | | | | | | | | | | | | | Automatic |
| | | | | | | | | | | | | | | Brightness |
| | | | | | | | | | | | | | | Control |
| | | | | | | | | | | | | | | Self- |
| | | | | | | | | | | | | | | Diagnostic |
| RDABCSDR | | | | | | | | | | | | | | Result |
| | 1 | 1 | ↑ | — | — | — | — | — | — | — | — | — | | Dummy read |
| | 1 | 1 | ↑ | — | D7 | D6 | 0 | 0 | 0 | 0 | 0 | 0 | | — |
| RDID1 | 0 | ↑ | 1 | — | 1 | 1 | 0 | 1 | 1 | 0 | 1 | 0 | (DAh) | Read ID1 |
| | 1 | 1 | ↑ | — | | | | | | | | | | Dummy read |
| | 1 | 1 | ↑ | — | ID17 | ID16 | ID15 | ID14 | ID13 | ID12 | ID11 | ID10 | | Read |
| | | | | | | | | | | | | | | parameter |
| RDID2 | 0 | ↑ | 1 | — | 1 | 1 | 0 | 1 | 1 | 0 | 1 | 1 | (DBh) | Read ID2 |
| | 1 | 1 | ↑ | — | — | — | — | — | — | — | — | — | | Dummy read |
| | 1 | 1 | ↑ | — | ID27 | ID26 | ID25 | ID24 | ID23 | ID22 | ID21 | ID20 | | Read |
| | | | | | | | | | | | | | | parameter |
| RDID3 | 0 | ↑ | 1 | — | 1 | 1 | 0 | 1 | 1 | 1 | 0 | 0 | (DCh) | Read ID3 |
| | 1 | 1 | ↑ | — | — | — | — | — | — | — | — | — | | Dummy read |
| | 1 | 1 | | — | ID37 | ID36 | ID35 | ID34 | ID33 | ID32 | ID31 | ID30 | | Read |
| | | | | | | | | | | | | | | parameter |

Timer initialization code is as follows:

```
Timer2
        Fire Every 100Hz
// Tell the Power Management Controller to disable
  // the write protection of the (Timer/Counter) registers:
  pmc_set_writeprotect(false);
  // Enable clock for the timer
  pmc_enable_periph_clk((uint32_t)t.irq);
  // Find the best clock for the wanted frequency
  clock = bestClock(frequency, rc);
  switch (clock) {
    case TC_CMR_TCCLKS_TIMER_CLOCK1:
      _frequency[timer] = (double)VARIANT_MCK / 2.0 / (double)rc;
      break;
    case TC_CMR_TCCLKS_TIMER_CLOCK2:
      _frequency[timer] = (double)VARIANT_MCK / 8.0 / (double)rc;
      break;
    case TC_CMR_TCCLKS_TIMER_CLOCK3:
      _frequency[timer] = (double)VARIANT_MCK / 32.0 / (double)rc;
      break;
    default: // TC_CMR_TCCLKS_TIMER_CLOCK4
      _frequency[timer] = (double)VARIANT_MCK / 128.0 / (double)rc;
      break;
  }
  // Set up the Timer in waveform mode which creates a PWM
  // in UP mode with automatic trigger on RC Compare
  // and sets it up with the determined internal clock as clock input.
  TC_Configure(t.tc, t.channel, TC_CMR_WAVE | TC_CMR_WAVSEL_UP_RC |
  // Reset counter and fire interrupt when RC value is matched:
  TC_SetRC(t.tc, t.channel, rc);
  // Enable the RC Compare Interrupt...
  t.tc−>TC_CHANNEL[t.channel].TC_IER=TC_IER_CPCS;
  // ... and disable all others.
  t.tc−>TC_CHANNEL[t.channel].TC_IDR=~TC_IER_CPCS;
Timer3
        Fire Every 2000Hz
Timer4
  Fire Every 100Hz
// Tell the Power Management Controller to disable
  // the write protection of the (Timer/Counter) registers:
  pmcsetwriteprotect(false);
  // Enable clock for the timer
  pmc_enable_periph_clk((uint32_t)t.irq);
  // Find the best clock for the wanted frequency
  clock = bestClock(frequency, rc);
  switch (clock) {
    case TC_CMR_TCCLKS_TIMER_CLOCK1:
      _frequency[timer] = (double)VARIANT_MCK / 2.0 / (double)rc;
      break;
    case TC_CMR_TCCLKS_TIMER_CLOCK2:
      _frequency[timer] = (double)VARIANT_MCK / 8.0 / (double)rc;
      break;
    case TC_CMR_TCCLKS_TIMER_CLOCK3:
      _frequency[timer] = (double)VARIANT_MCK / 32.0 / (double)rc;
      break;
    default: // TC_CMR_TCCLKS_TIMER_CLOCK4
      _frequency[timer] = (double)VARIANT_MCK / 128.0 / (double)rc;
      break;
  }
  // Set up the Timer in waveform mode which creates a PWM
  // in UP mode with automatic trigger on RC Compare
  // and sets it up with the determined internal clock as clock input.
  TC_Configure(t.tc, t.channel, TC_CMR_WAVE | TC_CMR_WAVSEL_UP_RC |
  // Reset counter and fire interrupt when RC value is matched:
  TC_SetRC(t.tc, t.channel, rc);
  // Enable the RC Compare Interrupt...
  t.tc−>TC_CHANNEL[t.channel].TC_IER=TC_IER_CPCS;
  // ... and disable all others.
  t.tc−>TC_CHANNEL[t.channel].TC_IDR=~TC_IER_CPCS;
```

The programming employed in the medical device 160 and application may include Atmel Studio tooling. Atmel Studio is an Integrated Development Environment (IDE) for writing and debugging AVR/ARM applications. Atmel Studio provides a project management tool, source file editor, simulator, assembler, and front-end for C/C++ programming, and on-chip debugging. Atmel Studio has a modular architecture, which allows interaction with third party software vendors. GUI plugins and other modules can be written and hooked to the system.

In addition to all the above requirements, there are a set of user requirements that are also needed. Table 14 provides a listing of these user requirements:

TABLE 14
User requirements

| | | |
|---|---|---|
| ID | Description | Comments |

Quality of Audio Output

| | | |
|---|---|---|
| 1.1 | Audio output should be amplified to a | Amplification factor of HealthyU speaker |
| | degree where user can listen to the heart | output is 6x. |
| | and lung sounds with ease. | |
| 1.2 | Device need to provide analog audio output | Audio Jack in HealthyU device can be |
| | for user to hear heart sounds | connected to a smart device using a |
| | | connector. This way user can listen to |
| | | analog audio/heart sounds from the smart |
| | | device. |
| 1.3 | ECG & PCG waveform from the device | HealthyU device shall be paired with smart |
| | need to be printable | device's Bluetooth via HealthyU app, print |
| | | option from smart device shall be used to |
| | | print ECG/PCG. |
| 1.4 | Heart sound sensed by device need to be | Ambient noise rejection algorithm is used |
| | immune to ambient noise | to remove ambient noise impact in |
| | | phonocardiogram |
| 1.5 | Clinician need to have access to heart | HealthyU app is capable of recording the |
| | sound data | heart sounds that can be shared with |
| | | clinician for diagnosis |

Visual Display Requirements

| | | |
|---|---|---|
| 2.1 | HealthyU device display needs to display | Display necessary information in the device |
| | the following, | display to achieve intended device usage. |
| | Time, Battery status, Firmware version | |
| | number and Label/icon of the vitals sent to | |
| | the HealthyU app | |
| 2.2 | Authentic and dependable visual | ECG and PCG waveforms shall be visible |
| | representation of ECG and PCG waveforms | on the smart device display in which |
| | | application software is installed. Display |
| | | should be a true representation of the |
| | | cadence of the ECG/PCG. Display speed |
| | | (Visual) and audio should be matched to |
| | | each other. |
| 2.3 | App should enable ECG & PCG amplitude | Signal amplitude adjustment is required to |
| | scaling option | view ECG/PCG from patients of different |
| | | physical constitution and cardiac |
| | | conditions, clearly enough to identify the |
| | | different phases and to derive conclusion. |
| 2.4 | Ability to visualize 3-4 cycles of ECG/PCG | Display module of software is designed to |
| | clearly without straining the eyes in the app | accommodate at least 3-4 cycles of ECG & |
| | screen. | PCG in the app device screen. |
| 2.5 | Ability to correlate “what you hear” to | Display speed should be matched with the |
| | “what you see” in real time | audio and the waveform traced on the |
| | | display should be in sync with the cadence |
| | | of the audio, so that the user is able to |
| | | correlate the display with the audio at any |
| | | moment during auscultation |
| 2.6 | Ability to save the displayed ECG/PCG | Application software shall record the |
| | waveform | ECG/PCG waveform automatically upon |
| | | receiving signal from HealthyU device. |
| | | User shall be able to save data so that it can |
| | | be reviewed by both the user and physician |
| 2.7 | Ability to quickly assess typical | Enables clinician to arrive at diagnosis and |
| | pathologies by estimating position and | treat the patients based on available vital |
| | intensity of different waveforms visually | data. |
| 2.8 | Display should be viewable under different | Device may be used indoors as well as |
| | lighting conditions | outdoors, under bright natural light |
| | | conditions as well as in dark areas, in |
| | | addition to artificially illuminated areas- the |
| | | display should be viewable without glare |
| | | under expected lighting conditions |
| 2.9 | App display should provide some method | Potential users require that ECG & PCG be |
| | of visually estimating the duration and | displayed on grids with the same spacing as |
| | amplitude of different ECG/PCG waveform | ECG papers. This may reduce the learning |
| | components | curve, since doctors are trained to read the |
| | | timing on such ECG papers by counting off |
| | | the number of lines traversed by the |
| | | waveform. |
| 2.10 | There should be options to maneuver | Screens can be changed from APP controls. |
| | between the various screens in the app | |

Usability Requirements

| | | |
|---|---|---|
| 3.1 | Device should be easy to use and should | Device usage should not involve complex |
| | involve minimum learning curve to explore | sequences of key presses. Keys and |
| | complete set of features | operations should be user friendly and need |
| | | to be designed for the lay persons as |
| | | intended user population |
| 3.2 | Device should be hand-held and operable | Device need to be easy to carry and to |
| | as a standalone device | operate. |
| 3.3 | Device should be operated by rechargeable | Device needs to be powered by |
| | batteries | rechargeable battery |
| 3.4 | Device should indicate whether the device | To alert user about the battery status and to |
| | is charging or if charging has been | enable intended device usage and operating |
| | completed | time, battery status need to be displayed in |
| | | the device |
| 3.5 | User should not have easy access to the | To prevent battery associated hazards, built |
| | battery compartment. | in battery need to be provided |
| 3.6 | Batteries should last at least for 5 hours of | To ensure home users can use the device for |
| | operation between successive recharges | at least 5 hours |
| 3.7 | Device should afford a simple cleaning | Recommended cleaning method need to be |
| | routine | described in IFU. Simple cleaning routine |
| | | suggested, making it easy for lay users |
| 3.8 | Button placement should be such that, any | Button placement should be available for |
| | movement to actuate the button should not | thumb finger operation at all positions |
| | occlude visibility of the display and button | |
| | usage should not introduce any motion | |
| | artifacts | |

Form Factor Requirements

| | | |
|---|---|---|
| 4.1 | Device should be easy to carry, handle, | Market feedback shows that the device |
| | position and operate. | weight should not exceed 200 grams, for |
| | | ease of handling and for carrying |
| 4.2 | Device construction should be rugged and | Temperature range at which the device can |
| | should be suited for rough usage; device | be transported, operated and stored shall be |
| | parts like display module, 7-lead ECG etc. | in compliance with IEC 60601-1 |
| | should be capable of withstanding | Should meet requirements for mechanical |
| | mechanical shocks due to fall, rough | shocks per IEC 60601-1-11 and IEC 60601- |
| | handling, varying temperatures in operating | 2-47 |
| | environment | |

Information Supplied by the Manufacturer

| | | |
|---|---|---|
| 5.1 | Device should be supported by | HealthyU shall come along with |
| | comprehensive information material to aid | comprehensive user manual/flipchart |
| | users in the usage of HealthyU system. | illustrating device handling, usage, caution |
| | | and precaution. |

Interface Requirements

| | | |
|---|---|---|
| 6.1 | Interface to stream, captured ECG/PCG in | Wireless communication |
| | any laptop or smart device in which has | |
| | application software | |
| 6.2 | Factory shall be able to update the | Troubleshooting can also be done from |
| | firmware. | remote location |

Safety Requirements

| | | |
|---|---|---|
| 7.1 | Device should not be operable during | Device shall enter standby mode when |
| | battery charging, unless the charging is | charging |
| | through safe means that isolates the device | |
| | from the mains | |
| 7.2 | Device shape and form factor should ensure | Device should have curved edges |
| | that it may not cause a safety hazard if it | |
| | accidentally falls on a person | |
| 7.3 | Device usage should not interfere with | Wireless Coexistence testing |
| | operation of other medical device 160s in | |
| | the vicinity and device should in turn | |
| | remain unaffected when used in normal | |
| | operating environment | |
| 7.4 | Device should not cause any safety hazard | The components used in device should |
| | to user or patient due to contact or due to | meet all quality standards so as to ensure |
| | the material used | biocompatibility safety per ISO 10993-1 |
| 7.5 | Device should not cause any safety hazard | IEC 60601-1, clause 8 |
| | to user or patient due to electrical faults | |
| 7.6 | Device should not cause any safety hazard | IEC 60601-1 clause 11.1 |
| | to user or patient due to thermal faults | |
| 7.7 | Device should not result in anomalous | Watchdog timer that is operated by |
| | behavior due to failure of the firmware. | controller clock may to be used to restart |
| | Should such an error occur, device should | the system automatically, in case of any |
| | shut down, restart or exit with error | failure |
| | message | |
| 7.8 | Should pass the safety requirement tests | Requirements identified in test plans, per |
| | | IEC 60601-1, IEC 6001-1-2 and other |
| | | applicable consensus standards listed in |
| | | section 4. |
| 7.9 | Device should be ingress protected to | Enclosures need to be designed to provide |
| | ensure reliable use in intended use | protection against harmful ingress per IEC |
| | environment | 60601-1-11, clause 11.6.5. |

Table 15 provides software requirements:

TABLE 15
Software Requirements

| | | |
|---|---|---|
| SRD ID | Specification | Rationale/Reference |

Functional Requirements

| SR001 | Operating system - Android | | Allow for use on smart |
|---|---|---|---|
| | | | device platforms |
| | | | A20 |
| SR002 | Minimum Screen resolution requirement of | | Display 3-4 cycles ECG & |
| | application software is 1280*800 pixels | | PCG clearly without eye |
| | | | strain |
| SR003 | Auto pairing with HealthyU device via Bluetooth | | Upon starting the app, |
| | Bluetooth icon is displayed on the screen during | | initiate Bluetooth pairing |
| | launch of software application | | with HealthyU device |
| | | | using Bluetooth icon |
| | | | displayed on the |
| | | | application screen. |
| | | | A6 |
| SR004 | Bluetooth icon with toggle button to indicate | | Allow user to confirm |
| | connectivity to HealthyU device | | Bluetooth connectivity |
| | Toggle between blue and grey colors | | between the application |
| | Blue indicates Bluetooth connectivity has | | and the HealthyU device, |
| | been established | | as well as re-initiate |
| | Grey indicates Bluetooth connectivity has not | | connectivity if lost. |
| | been established | | A9, A14, A15, A23 |
| | Tapping on grey icon initiates Bluetooth | | |
| | connection with HealthyU device | | |
| SR005 | If there is only a single HealthyU device, that may | | User to select which |
| | be selected automatically. | | HealthyU device to be |
| | If there is more than one device detected | | paired |
| | within Bluetooth Low Energy (BLE) range, all | | |
| | the detected devices are listed by name and | | |
| | MAC ID. User can select one of them. The | | |
| | mac id to be labelled on the device | | |
| SR006 | User can set default HealthyU device which | | Enable default Bluetooth |
| | can be auto paired every time the app starts, | | pairing with user's |
| | provided the device is ON and is in range. | | HealthyU device |
| | The default device may be changed at any time | | |
| | by the user. | | |
| SR007 | HD Medical & HealthyU logo displayed on top | | Identify app as a HD |
| | left corner of app screen | | Medical product |
| SR008 | Available device modes shall be: | | Allow user to select |
| | Steth (HS & ECG Channel 1) | | between device modes that |
| | 7-Channel ECG | | provide STETH (HS and |
| | | | single-channel ECG), 7- |
| | | | channel ECG functionality |
| | | | via separate app screens |
| SR009 | STETH screen - PCG & Channel 1 ECG | | Provide a main viewing |
| | Auscultation waveform plotting shall be displayed | | screen upon starting the |
| | on landing screen, following successful Bluetooth | | app. |
| | pairing with HealthyU device. | | |
| SR010 | 7-channel Screen | | Allow user to view 7- |
| | | | channel ECG in the app |
| SR011 | Option to rename the ECG channels labels using | | Allow user to rename the |
| | settings | | ECG labels |
| SR012 | Heart Sound data (PCG) shall be depicted as a blue | | View heart sound data as |
| | waveform (default). Waveform color can be | | PCG waveform |
| | changed by the user via the user-configurable | | |
| | Settings | | |
| SR013 | ECG waveform shall be depicted as a green | | View ECG waveform |
| | waveform (default) with an ECG grid which | | |
| | appears by default. | | |
| SR014 | In HealthyU auscultation screen, indications for | | Allow user to measure |
| | measurements of waveform by default are given as, | | ECG & PCG waveforms |
| | major axis = 5 mm | | manually |
| | Duration: 25 mm/S | | |
| | Amplitude: 10 mm/mV | | |
| | Above items may be indicated graphically. | | |
| SR015 | All 7 channel ECG waveforms shall be | | Allow user to view all 7 |
| | accommodated in a single screen. | | channel ECG waveforms |
| | | | on a single screen |
| SR016 | Vertical scroll bar in right side of the screen | | For user to view specific |
| | | | waveform by scrolling up |
| | | | or down throughout the 7- |
| | | | channel screen |
| SR017 | Add study button is present in both Steth & 7- | | To associate ECG & PCG |
| | channel screen to add user details of the present | | data with respective user |
| | study | | |
| SR018 | User name and id of the present study may be | | To associate recorded data |
| | displayed on the app screen. | | with correct user |
| SR019 | Record button present in both Steth & 7-channel | | Function to enable the |
| | screen to initiate recording | | saving the ECG & PCG |
| | | | waveforms |
| SR020 | The recording duration measured in seconds can | | User configurable setting |
| | be configured (10 sec, 20 sec, 30 sec up to 120 | | |
| | sec.) by user via settings. Default is 10 seconds. | | |
| SR021 | While recording is in-progress all other buttons in | | Disable all other app |
| | the screen shall be inactive, the record button | | button functions during |
| | should get replaced by a progress bar that may | | recording to avoid |
| | indicate the progress of the recording. | | unintentional interruption |
| | | | A16 |
| SR022 | Freeze button in both Steth & 7-channel screen to | | Function to |
| | enable screen capturing | | instantaneously screen |
| | | | capture the live waveforms |
| SR023 | Screenshot may be saved to patient record/worklist | | Button to take screenshot |
| | | | of the real time displayed |
| | | | data |
| SR024 | Add/exit study button is present in both Steth and | | To associate recorded data |
| | 7 channel screen for user to switch between | | with correct user |
| | multiple user profiles and enable user to store or | | |
| | link data with correct user profile. | | |
| SR025 | A toggle icon to switch between Steth screen and | | For user to view the 7 |
| | 7-channel screen in the app screen | | channel ECG screen & |
| | | | Steth screen |
| SR026 | Once recording duration is completed, user | | Allows user to preview |
| | may be taken to preview screen, in the | | both |
| | preview screen, data of Steth (PCG and | | Steth screen data and 7 |
| | Channel 1 ECG), or 7 Channel ECG screen | | channel ECG data together |
| | may be displayed. | | |
| SR027 | After checking the preview screen, user can | | Enables user to save or |
| | either choose to save or discard the data | | discard the data displayed |
| | shown in preview using save or discard | | in the preview screen |
| | button displayed in the preview screen. | | |
| SR028 | While preview screen is in active state, recording | | Enables user to save or |
| | of data may not occur & the recorded waveforms | | discard the data displayed |
| | may be displayed on the Steth or 7 channel ECG | | in the preview screen |
| | screen as recorded. | | |
| SR029 | If user chooses to save the data displayed in the | | Provide “save” button to |
| | preview screen, user may be directed to choose | | allow user to save |
| | whether the data has to be saved to a new patient | | recorded data for either a |
| | or existing patient | | new or existing patient |
| SR030 | New Patient: | | Allow user to create new |
| | Add new patient profile. After saving current | | patient profile when |
| | recording, the program shall lead to Patient | | saving recorded data for a |
| | History containing list of recordings. | | new patient |
| SR031 | Existing Patient: | | Allow user to choose from |
| | Display a screen with list of patients. User shall be | | existing patient profiles |
| | able to search and identify a patient to save the | | when saving recorded data |
| | current recording. | | for an existing patient |
| SR032 | Discard the temporarily stored recording and move | | Allow user to discard |
| | to the Steth screen so that the user can proceed to | | current recording and |
| | conduct a new recording. | | initiate new recording |
| | Require user confirmation before performing this | | |
| | action. | | |
| SR033 | Viewing patient record from patient files: | | Retrievable and accessible |
| | User shall be able to view the history of patient | | patient records |
| | recording including patient personal data and | | |
| | ECG, PCG and vitals by tapping the patient | | |
| | history/worklist button. | | |
| SR034 | While viewing recorded data from the worklist, the | | Allow user to listen to |
| | PCG sound should be audible on the user device | | playback of PCG sound on |
| | (example: Tablet, Mobile or laptop). | | their device |
| SR035 | Settings button in Steth & 7-channel screen | | To access settings screen |
| SR036 | Button to access patient record in both Steth & 7- | | To access patient records |
| | channel screen | | from main screen |
| SR037 | A device info button to provide smart device | | Smart device |
| | specification | | specifications |
| SR038 | Device placement guidelines to be displayed in | | To guide user |
| | app on the splash screen | | |
| SR039 | Firmware version displayed in app screen | | Firmware information |
| | | | A27 |
| SR040 | HealthyU device battery status is displayed in app | | To enable user to monitor |
| | | | device battery status while |
| | | | device is in use |
| SR041 | Select posture & select position screen appears | | To acquire information on |
| | after recording duration | | user posture and device |
| | | | positioning |
| SR042 | Permission requesting smart device resources | | To enable app's access to |
| | window appear when the app is launched first time | | smart device, android |
| | | | requirement |
| SR043 | Patient report is generated in PDF format. PDF has | | To enable sharing and |
| | both PHI and PII | | printing |
| SR044 | Receives channel 1 (lead I), channel 2 (lead II) and | | To display 7-channel ECG |
| | channel 7 (lead V) data from firmware | | |
| SR045 | Augmented waveforms (channel 4 (aVF), channel | | To display 7-channel ECG |
| | 5 (aVL),channel 6 (aVR) and channel 3 (Lead III) | | |
| | are derived in software | | |
| SR046 | Channel | Configuration | Channel and its |
| | Channel 1 | LA − RA | configuration |
| | Channel 2 | LL − RA | |
| | Channel 3 | Channel 2 − Channel 1 | |
| | Channel 4 | −(Channel 1 + Channel 2)/2 | |
| | Channel 5 | Channel 1 − channel 2/2 | |
| | Channel 6 | Channel 2 − Channel 1/2 | |
| | Channel 7 | Electrode 1 − | |
| | | (RA + LA + Electrode 2)/3 | |

| | | |
|---|---|---|
| SR047 | Heart rate is calculated after the recorded signal is | To display heart rate in |
| | saved by the user | patient file |
| SR048 | Heart rate is displayed in patient file generated in | To display heart rate in |
| | PDF format | patient file |
| SR049 | HR filter shall be used for HR calculation | To calculate heart rate |
| | | A19 |
| SR050 | ECG and PCG can be replayed after saving them | To replay saved ECG and |
| | | PCG |
| SR051 | First time users need to register their device | To generate customer code |
| | following the onscreen instruction. | and database |
| | | A10 |
| SR052 | If smart device battery is low, a message is | To inform user to ensure |
| | displayed, recommending user to charge the smart | continuous device usage |
| | device | A31 |
| SR053 | Amplitude scaling option in steth screen | To vertically zoom PCG |
| | | and channel 1 ECG |
| SR054 | Heart rate range is between 30 to 250 bpm | To use for patients ≥10 Kg |
| SR055 | Accuracy of heart rate measurement need to be ±5 | To ensure integrity |
| | bpm | |
| SR056 | App should be designed for lay users | To ensure easy usability |
| | | experience for lay users |
| | | A29 |
| SR057 | Registration module for user to register their | Product maintenance |
| | HealthyU device with HD Medical, Inc. | |
| SR058 | Device placement position (Aortic, Pulmonary | Map the electrode |
| | Tricuspid, Mitral, V1 to V6) and user posture | locations with ECG |
| | (sitting, standing, supine) information to be | acquired and user posture |
| | entered by user for each recording | |

Minimum requirements of HealthyU app to run on Android platforms

| | | |
|---|---|---|
| SR044 | Processor requirement is Snapdragon 425 or | Enable app to achieve its |
| | equivalent or faster processor family | intended use |
| SR045 | Memory requirement is minimum 2 GB RAM | To allow app performance |
| SR046 | Storage requirement is 10 GB or above | To store patient data |
| | | locally in the smart device |
| | | A13 |
| SR047 | Screen size - 8 inch | Allow data reproducibility |
| | | in real time |
| SR048 | Bluetooth 5.0 (BLE) & above | Allow auto Bluetooth |
| | | pairing of device & app |
| SR049 | Android 9.0 or above | Enable app to achieve its |
| | | intended use |
| SR050 | Smart device with audio jack | To playback the recorded |
| | | PCG |
| SR051 | Wi-Fi & GPS to enable user registration & to | Enable app to achieve its |
| | download app from play store | intended use |

Patient Profile

| | | |
|---|---|---|
| SR101 | Provide general patient details - First name, Last | Allow user to enter patient |
| | name, patient id, Gender, Date of birth, Contact | information. |
| | Information. All these items are mandatory. | A33 |
| SR102 | Date of Birth should be entered using Calendar | Allow user to enter date of |
| | option. Invalid dates should be indicated | birth using calendar |
| | appropriately. Manual entering is not allowed. | selection. |
| SR103 | Age is calculated from the date of birth and | Allow user to view age of |
| | displayed. It shall not be editable directly. | patient as calculated from |
| | | date of birth. |
| SR104 | Gender options - Male, Female and Other shall be | Allow gender selections as |
| | available in the form of Radio buttons. | male, female, or other. |
| SR105 | Remainder of fields for patient address, phone and | Allow text entry for |
| | email information are text data. | patient contact info fields. |
| SR106 | Fields such as height, weight, blood pressure, | Allow for optional fields |
| | temperature, hemoglobin, blood glucose, oxygen | that do not require user |
| | saturation under history section are optional fields. | data entry. |
| | User can keep these fields as blank and save | |
| | Patient's profile. | |
| SR107 | Patient id to be set by user | Used as unique record |
| | | identifier |

Patient Files & Demo Video

| | | |
|---|---|---|
| SR201 | Demo video of how to operate HealthyU | Allow user to easily |
| | application is provided | understand HealthyU |
| | | operation or usage |
| SR202 | Patient Files: | List existing saved patient |
| | Patients with latest saved recordings appear on top. | recordings for selection |
| | The search option is available to look for a patient | and allow ability to search |
| | by entering name or patient ID or contact number. | on patient ID. |
| SR203 | On choosing a patient, the history of all recordings | Allow user to view patient |
| | for that patient, identifiable by their timestamp, | history and identify |
| | shall be produced. The most recent recordings | recordings by timestamp |
| | shall appear on top. | |
| SR204 | On tapping patient record, the particular | Allow user to view |
| | records may be displayed on the viewing | specific recording by |
| | screen. | tapping patient record |
| SR205 | The patient name & time stamp shall appear on the | Allow user to easily view |
| | top row of the patient record. | patient name and |
| | | timestamp for each |
| | | recording |

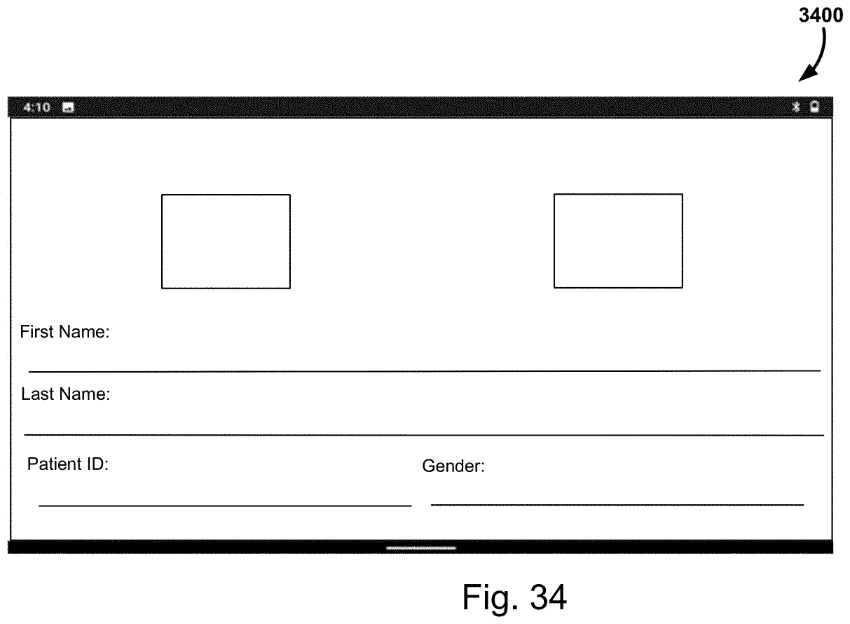

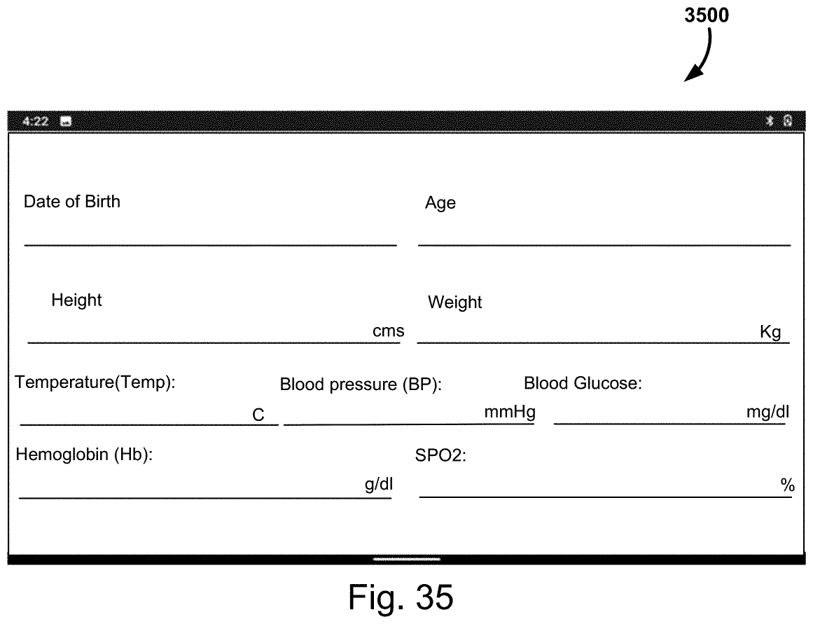

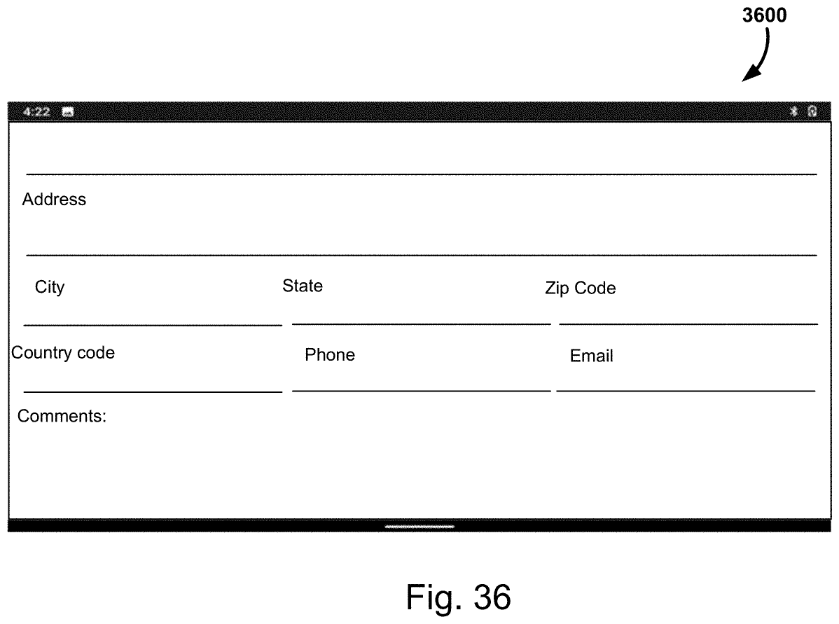

A patient profile is generated for each user of the system. When saving a recording to a new patient from the Preview screen, a patient profile screen, as seen in FIGS. 34-36, at 3400, 3500, and 3600, respectively, is displayed. This screen consists of three sections: 1) Profile—This section has fields for entering the general details of the patient like Name, Date of Birth, Gender, Contact details and medical history. Patient Id shall be automatically generated once the registration process is completed; 2) History—The medical history of the patient can be stored in this section. This is optional information to be stored in the app for accurate prognosis by clinician; and 3) Contact Details—User contact details such as phone number, address are entered here.

Turning back to the hardware components of the medical device 160, The microcontroller is a Flash microcontroller based on the high performance 32-bit ARM Cortex-M3 RISC processor. It operates at a maximum speed of 84 MHz and features up to 512 Kbytes of Flash and up to 100 Kbytes of SRAM. The peripheral set includes a Highspeed USB Host and Device port with embedded transceiver, an Ethernet MAC, 2 CANs, a Highspeed MCI for SDIO/SD/MMC, an External Bus Interface with NAND Flash Controller (NFC), 5 UARTs, 2 TWIs, 4 SPIs, as well as a PWM timer, 3-channel general-purpose 32-bit timers, a low-power RTC, a low power RTT, 256-bit General Purpose Backup Registers, a 12-bit ADC. The microcontroller architecture is specifically designed to sustain high-speed data transfers. It includes a multi-layer bus matrix as well as multiple SRAM banks, PDC and DMA channels that enable it to run tasks in parallel and maximize data throughput. The device operates at 3.3V and is available in 144-lead LQFP, LFBGA packages.

Pin mapping for this type of microcontroller may be seen below in Table 16:

TABLE 16
Microcontroller Pin Mapping

| SAM3X Pin Name | Mapped Pin Name | As Per Schematic |
|---|---|---|
| PA8 | RX0 | RXD0 |
| PA9 | TX0 | TXD0 |
| PB25 | Digital Pin 2 | BT_AUTORUNn |
| PC28 | Digital Pin 3 | CHARG_STATUS |
| connected to both | Digital Pin 4 | ECG_SPIO_CSn |
| PA29 and PC26 | | |
| PC25 | Digital Pin 5 | DRDYBn |
| PC24 | Digital Pin 6 | BUZZER |
| PC23 | Digital Pin 7 | C23 (TP) |
| PC22 | Digital Pin 8 | LCD_RESET |
| PC21 | Digital Pin 9 | LCD_DC |
| PA28 and PC29 | Digital Pin 10 | LCD_CS |
| PD7 | Digital Pin 11 | LCD_MOSI |
| PD8 | Digital Pin 12 | Freeze_SW |
| PB27 | Digital Pin 13/Amber | LCD_SPCK |
| | LED “L” | |
| PD4 | TX3 | TXD3 |
| PD5 | RX3 | RXD3 |
| PA13 | TX2 | TXD2 |
| PA12 | RX2 | RXD2 |
| PA11 | TX1 | BT_UART_TXD |
| PA10 | RX1 | BT_UART_RXD |
| PB12 | SDA | SPO2_SDA |
| PB13 | SCL | SPO2_SCL |
| PB26 | Digital Pin 22 | PHP_RSTn |
| PA14 | Digital Pin 23 | NC |
| PA15 | Digital Pin 24 | NC |
| PD0 | Digital Pin 25 | MFIO_2 |
| PD1 | Digital pin 26 | RESET_2 |
| PD2 | Digital Pin 27 | D2 (TP) |
| PD3 | Digital Pin 28 | D3 (TP) |
| PD6 | Digital Pin 29 | D6 (TP) |
| PD9 | Digital Pin 30 | NC |
| PA7 | Digital Pin 31 | PA7 (TP) |
| PD10 | Digital Pin 32 | D10 (TP) |
| PC1 | Digital Pin 33 | EN_PCG_3V3A |
| PC2 | Digital Pin 34 | uP_Force Off |
| PC3 | Digital Pin 35 | EN_SPO2_1V8D |
| PC4 | Digital Pin 36 | uP_ON/OFF |
| PC5 | Digital Pin 37 | AMP_SHUTN |
| PC6 | Digital Pin 38 | C6 (TP) |
| PC7 | Digital Pin 39 | C7 (TP) |
| PC8 | Digital Pin 40 | C8 (TP) |
| PC9 | Digital Pin 41 | C9 (TP) |
| PA19 | Digital Pin 42 | NC |
| PA20 | Digital Pin 43 | NC |
| PC19 | Digital Pin 44 | NC |
| PC18 | Digital Pin 45 | NC |
| PC17 | Digital Pin 46 | NC |
| PC16 | Digital Pin 47 | NC |
| PC15 | Digital Pin 48 | NC |
| PC14 | Digital Pin 49 | NC |
| PC13 | Digital Pin 50 | NC |
| PC12 | Digital Pin 51 | NC |
| PB21 | Digital Pin 52 | NC |
| PB14 | Digital Pin 53 | NC |
| PA16 | Analog In 0 | A0 (TP) |
| PA24 | Analog In 1 | A1 (TP) |
| PA23 | Analog In 2 | A2 (TP) |
| PA22 | Analog In 3 | A3 (TP) |
| PA6 | Analog In 4 | EN_LCD_3V3D |
| PA4 | Analog In 5 | BATT_MONITOR |
| PA3 | Analog In 6 | ANR_AFE |
| PA2 | Analog In 7 | PCG_AFE |
| PB17 | Analog In 8 | EN_BT_ECG_3V3D |
| PB18 | Analog In 9 | EN_SPO2_3V3D |
| PB19 | Analog In 10 | RESET |
| PB20 | Analog In 11 | MFIO |
| PB15 | DAC0 | DAC0 (CANRX1) (TP) |
| PB16 | DAC1 | DAC1 |
| PA1 | CANRX | CANRX0 |
| PA0 | CANTX | CANTX0 |
| PA17 | SDA1 | TEMP_SDA |
| PA18 | SCL2 | TEMP_SCL |
| PC30 | LED “RX” | C30 (TP) |
| PA21 | LED “TX” | PA21 (TP) |
| PA25 | (MISO) | ECG_SPI0_MISO |
| PA26 | (MOSI) | ECG_SPI0_MOSI |
| PA27 | (SCLK) | ECG_SPI0_CLK |
| PA28 | (NPCS0) | LCD_CS |
| PB23 | (unconnected) | NC |
| PB11 | ID | NC |
| PB10 | VBOF | NC |
| PC10 | | C10 (TP) |
| PC30 | | C30 (TP) |

The Microcontroller product has several types of power supply pins: VDDIO pins: Power the peripherals I/O lines: voltage range +3.3 v; VDDIN pin: Powers the voltage regulator: voltage range +3.3 v; VDDBU pin: Powers the Slow Clock oscillator and a part of the System Controller; voltage range of +3.3V. VDDBU should be supplied before or at the same time as VDDIO and VDDCORE; VDDUTMI pin: Powers the UTMI+ interface: voltage range of 3.3V; VDDANA pin: Powers the ADC and DAC cells; voltage range of 3.3V; VDDOUT pin: Output of the voltage regulator; VDDCORE pins: Power the core, the embedded memories and the peripherals; pin: Powers the PLL A, UPLL and 3-20 MHz Oscillator.

Ground pins GND are common to VDDCORE and VDDIO pins power supplies. Separated ground pins are provided for VDDBU, VDDPLL, VDDUTMI and VDDANA. These ground pins are respectively GNDBU, GNDPLL, GNDUTMI and GNDANA.

The Power Supply Controller controls the power supplies of the processor and peripherals via Voltage regulator control. The Supply Controller has its own reset circuitry and is clocked by the 32 kHz Slow clock generator. The reset circuitry is based on a zero-power power-on reset cell. The zero-power power-on reset allows the Supply Controller to start properly.

The Power-on-Reset monitors VDDBU. It is always activated and monitors voltage at start up but also during power down. If VDDBU goes below the threshold voltage, the entire chip is reset. The Brownout Detector monitors VDDCORE. It is active by default. It can be deactivated by software through the Supply Controller (SUPC_MR). It is especially recommended to disable it during low-power modes such as wait or sleep modes. If VDDCORE goes below the threshold voltage, the reset of the core is asserted. The Supply Monitor monitors VDDUTMI. It is not active by default. It can be activated by software and is fully programmable with 16 steps for the threshold (between 1.9V to 3.4V). It is controlled by the Supply Controller (SUPC). A sample mode is possible. It allows to divide the supply monitor power consumption by a factor of up to 2048.

Clock inputs include: A Low Power 32.768 KHz Slow Clock Oscillator with bypass mode; A Low Power RC Oscillator; A 3 to 20 MHz Crystal or Ceramic Resonator-based Oscillator, which can be bypassed; A 480 MHz UTMI PLL, providing a clock for the USB High Speed Controller; A 96 to 192 MHz programmable PLL (input from 8 to 16 MHz), capable of providing the clock MCK to the processor and to the peripherals.

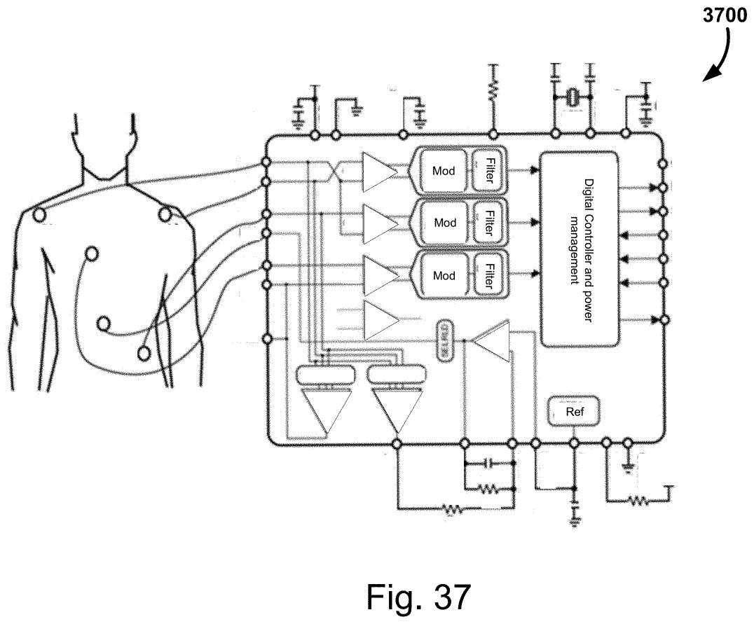

In the 5-Lead ECG shown in FIG. 37 at 3700, the medical device 160 uses the Common-Mode Detector to measure the common-mode of the system by averaging the voltage of input pins IN1, IN2 and IN3, and uses this signal in the right-leg drive feedback circuit. The output of the RLD amplifier is connected to RL through IN4 to drive the common-mode of the system. The Wilson Central Terminal is generated by the medical device 160 and is used as a reference to measure the chest electrode, V1. The chip uses an external 4.096 MHz crystal oscillator connected between the XTAL1 and XTAL2 pins to create the clock source for the device.

The ECG signals are captured using 5 electrodes placed on circular board. OPA2335 & High pass filter rectifies the signal capture by electrodes and medical device 160 filters the signal and send to microcontroller through SPI channel for digital signal processing. The Serial Peripheral Interface (SPI) circuit is a synchronous serial data link that provides communication with MEDICAL DEVICE 160 devices in Master or Slave Mode. It also enables communication between controller with analog front end for biopotential. Master Out Slave In (MOSI): This data line supplies the output data from the master (microcontroller) shifted into the input(s) of the slave (ADS12923). Master In Slave Out (MISO): This data line supplies the output data from a slave (medical device 160) to the input of the master (microcontroller). There may be no more than one slave transmitting data during any particular transfer. Serial Clock (SPCK): This control line is driven by the master and regulates the flow of the data bits. The master may transmit data at a variety of baud rates; the SPCK line cycles once for each bit that is transmitted. Slave Select (NSS): This control line allows slave to be turned on and off by hardware. Pin mapping for the SPI is shown on Table 17 below:

TABLE 17
SPI Pin Mapping

| MEDICAL | | MICRO- | |
|---|---|---|---|
| DEVICE | | CONTROLLER | |
| 160 pin | Function | pin | Function |
| 19 | Serial Data Output | PA25 | ECG SPI0 MISO |
| | (SPI0) | | |
| 18 | Serial Data Input | PA26 | ECG SPI0 MOSI |
| | (SPI0) | | |
| 17 | Serial Clock (SPI0) | PA27 | ECG SPI0 Clock |
| 16 | Chip Select Bar | PA29 | ECG SPI0 Chip |
| | (SPI0) | | Select |

OPA2335 is a CMOS operational amplifier used as a buffer amplifier to provide low offset voltage 5uv(MAX). OPA2335 is operates at 3.3 v(VCC_3 v3A). The amplified signal is fed into high pass filter to rectify the signal.

Medical device 160 analog front end connect with ARM core over SPI channel. Sampling rate of medical device 160 is 160±5 Hz. Basic features of MEDICAL DEVICE 160 implemented for our applications are: Three High-Resolution Digital ECG Channels; Data Rate: Up to 25.6 ksps; Built-In Oscillator and Reference; AC and DC Lead-Off Detection. ECG Pin mapping is illustrated below in Table 18:

TABLE 18
ECG Pin Mapping
ECG PIN Mapping

| | Mapped Pin | As Per |
|---|---|---|
| SAM3X Pin Name | Name | Schematic |
| PA25 | (MISO) | ECG_SPI0_MISO |
| PA26 | (MOSI) | ECG_SPI0_MOSI |
| PA27 | (SCLK) | ECG_SPI0_CLK |
| connected to | Digital Pin 4 | ECG_SPI0_CSn |
| both PA29 and PC26 | | |
| PC25 | Digital Pin 5 | DRDYBn |

Layout consideration & supply filtering for the medical device 160 includes: Used 0.1 uF/16V ceramic bypass capacitor from Analog Supply Voltage (VDD) to ground as close as possible to the pin; Used 0.1 uF/16V ceramic bypass capacitor from Digital Supply Voltage (VDDIO) to ground as close as possible to the pin; Used a low ESR 1 uF/16V bypass capacitor from CVREF pin to ground as close as possible to the pin; Used a low ESR 0.1 uF/16V bypass capacitor from RLDREF pin to ground as close as possible to the pin; The SPI signal traces routed close together; 49.9E Series resistors are placed at SDO and DRDYB pins of medical device 160.

A low-power rail-to-rail input/output operational amplifier specifically designed for portable applications is leveraged. The input common-mode voltage range extends beyond the supply rails for maximum dynamic range in low-voltage systems. The amplifier output has rail-to-rail performance with high-output-drive capability, solving one of the limitations of older rail-to-rail input/output operational amplifiers. This rail-to-rail dynamic range and high output drive make the amplifier ideal for buffering analog-to-digital converters. The operational amplifier has 6.4 MHz of bandwidth and 1.6 V/μs of slew rate with only 500 μA of supply current, providing good ac performance with low power consumption. Three members of the family offer a shutdown terminal, which places the amplifier in an ultralow supply current mode (IDD=0.3 μA/ch). While in shutdown, the operational-amplifier output is placed in a high-impedance state. DC applications are also well served with an input noise voltage of 11 nV/✓Hz and input offset voltage of 100 μV.

A mono bridged audio power amplifier capable of delivering power into a 3Ω load with less than 10% THD is also leveraged. To conserve power in applications, the LM4871's micropower shutdown mode (IQ=0.6 μA, typ) is activated when VDD is applied to the SHUTDOWN pin. It is specially designed to provide high power, high fidelity audio output. They require few external components and operate on the supply voltage of PCG_V3VA.

The communication between Healthy U App & Healthy U device is achieved by Bluetooth BL-652-SA module. It is v5.0 single mode Bluetooth module. The supply voltage for Bluetooth module is BT_ECG 3V3D. The module specifications are provided in Table 19:

TABLE 19
Bluetooth Module Specifications

| | | |
|---|---|---|
| Wireless | Bluetooth | V 5.0 - Single-Mode |
| Specification | Frequency | 2.402-2.480 GHz |
| | Transmit Power | +4 dBm (maximum). −20 dBm(minimum) |
| | Receive Sensitivity | −96 dBm (typical) |
| Host Interface | UART Interface | TX, RX, CTS, RTS |
| | | From 1200 bps to 1 Mbps |
| | I2C Interface | 1 I2C Interface (up to 400 kbps) |
| Profiles | Services Supported | Laird's smart BASIC firmware supports the |
| | | following: |
| | | Central Mode |
| | | Peripheral Mode |
| | | Custom Series |
| | Nordic SDK v3x0 | Any exposed within the related Nordic soft device |
| | | (application development to be done by OEM) |
| Programmability | smart BASIC | On-board programming language similar to BASIC. |
| | smart BASIC application | Via UART |
| | download | Via Over-the-Air (if SIO_02 pin is pulled high |
| | | externally) |
| Power | Voltage | 1.8-3.6 V - Internal DCDC converter or LDO |
| | Active Modes Peak | Advertising mode 7.5 mA peak Tx (with DCDC) |
| | Current (for maximum Tx | Connecting mode 5.4 mA peak Tx (with DCDC) |
| | power +4 dBm) - Radio | |
| | only | |
| | Active Modes Peak | Advertising mode 2.7 mA peak Tx (with DCDC) |
| | Current (for Tx Whisper | Connecting mode 5.4 mA peak Tx (with DCDC) |
| | mode2 power −40 dBm) - | |
| | Radio only | |
| | Active Modes Average | Depends on many factors |
| | Current | |
| | UltraLow Power Modes | Standby Doze 2.0 uA typical |
| | | Deep Sleep 400 nA |

Smart BASIC runtime engine firmware checks for the status of nAutoRUN during power-up or reset. The nAutoRUN pin detects if the BL652 module should power up into Interactive/Development Mode or Self-contained Run Mode. If nAutoRUN pin is at 0V and an “autorun” application exists in the modules file system, then firmware executes the smart BASIC application script automatically. The firmware may not execute the smart BASIC application script automatically. It allows developers to debug/change the script. nAutoRUN pin default state made HIGH by using 10K pullup resister connected to BT_ECG_3V3D. This pin is also connected with MICROCONTROLLER for making the nAutoRUN pin High/Low state using software.

Integrated chip antenna performance is sensitive to host PCB. It is critical to locate the antenna on the edge of the host PCB (or corner) to allow the antenna to radiate properly. Ensure there is no copper in the antenna keep-out area on any layers of the host PCB. Keep all mounting hardware and metal clear of the area to allow proper antenna radiation. For best antenna performance, place the module on the edge of the host PCB, preferably in the corner with the antenna facing the corner.

The charger is a cost-effective fully-integrated battery charger for Li-Polymer batteries. It uses current, voltage and temperature control loops to regulate the charge current. The high input voltage, up to 28V. A typical charge cycle includes trickle, constant-current (CC) and constant-voltage (CV) charge modes. The CC-mode current is programmable up to 600 mA with an external resistor. The voltage across the external resistor is also used to monitor the actual charge current. The constant voltage is fixed at 4.2V with 0.7% accuracy over a −20° C. to 70° C. temperature range. The trickle-mode current is preset to 20% of the CC-mode current when the battery voltage is lower than the trickle-mode threshold. The end-of-charge (EOC) current threshold is preset to 10% of the CC-mode current to save the board space and cost. A charge current thermal foldback feature limits the charge current when the IC internal temperature rises to a preset threshold.

The charger also protects the system with its input over-voltage protection (OVP) feature. In addition, the charger has a 2.6V falling power-on-reset (POR) threshold, making it perfect to work with current limited power supplies. Three indication pins (PPR, CHG and FAST) can be simply interfaced to a microcontroller. When no power supply is connected, or when disabled, the charger draws less than 1.0 μA leakage current from the battery.

The charger requires only two external capacitors and one resistor to build a fully functional charger for applications. Its ultrahigh-accuracy (±0.7%) output voltage and temperature-limited charging current offer additional battery safety during charging. The CC-mode current can be programmed with an external resistor (RISET). The voltage across this resistor is proportional to the charge current, so the system can monitor the charge current during the whole charge cycle. The EOC current threshold is preset to 10% of the CC-mode current. For a deeply discharged battery with a voltage lower than 2.7V, the charger charges the battery with a trickle-mode current, which is 20% of the CC-mode current.

A current shunt and power monitor with I2C interface is also leveraged. This module monitors both shunt voltage drop and bus supply voltage. Current sensor circuit is given in HealthyU schematic document, 0302-50012-000.

The Over voltage protection circuit (OVP) uses a P-channel MOSFET (SI2305CDS-T1-GE3) for load switching. It has a low RDS (on)=0.048 Ohm characteristics. 5.1V Zener diode (BZT585B5V1TQ-7) & PNP transistor (MMBT3906LP-7B) is used for controlling the MOSFET gate. If the battery voltage is exceeding more than 5.1V, Zener breaks down & this may turn OFF the transistor. A low at gate pin may switch off the MOSFET so there is no current flow between source to drain.

The power supply circuit is low-noise, linear regulators that deliver up to 500 mA of output current with only 10.5μVRMS of output noise from 10 Hz to 100 kHz. These regulators maintain ±1% output accuracy over a wide input voltage range, requiring only 100 mV of input-to-output headroom at full load. The 365 μA no-load supply current is independent of dropout voltage. The power supply circuit have nine, pin-selectable output voltages apart from nine we have selected the 3.3V for our application. It includes the programmable output soft-start rate, output overcurrent, and thermal overload protection. It is offered in an 8-pin TDFN package. The power supply circuit includes the following features: 1.7V to 5.5V Input Voltage Range; 0.6V to 5.3V Output Voltage Range; 10.5μVRMS Output Noise, 10 Hz to 100 kHz; 365 μA Operating Supply Current; 70 dB PSRR at 10 kHz; 500 mA Maximum Output Current; ±1% DC Accuracy Over Load, Line, and Temperature; 100 mV (Max) Dropout at 500 mA Load (3.6VIN); <0.1 μA Shutdown Supply Current; Stable with 2 μF (Min) Output Capacitance; Programmable Soft-Start Rate; Overcurrent and Overtemperature Protection, and; Output-to-Input Reversed Current Protection.

Lastly, the unit construction materials are provided in Table 20:

TABLE 20
Device Construction Materials

| S. No. | Part Name | Material Name |
|---|---|---|
| 1 | Display side casing | ABS: SD-0150 |
| 2 | Protective display | Poly carbonate |
| | cover | |
| 3 | Chest side casing | ABS: SD-0150 |
| 4 | On/Off button | ABS: SD-0150 |
| 5 | Finger electrode | Brass IS 4413 plated with |
| | | Nickel and GOLD plating |
| 6 | Chest electrode | Brass IS 4413 plated with |
| | | Nickel and GOLD plating |
| 7 | Diaphragm Sleeve | PVC |
| 8 | Diaphragm | Poly carbonate |

### Backend Server

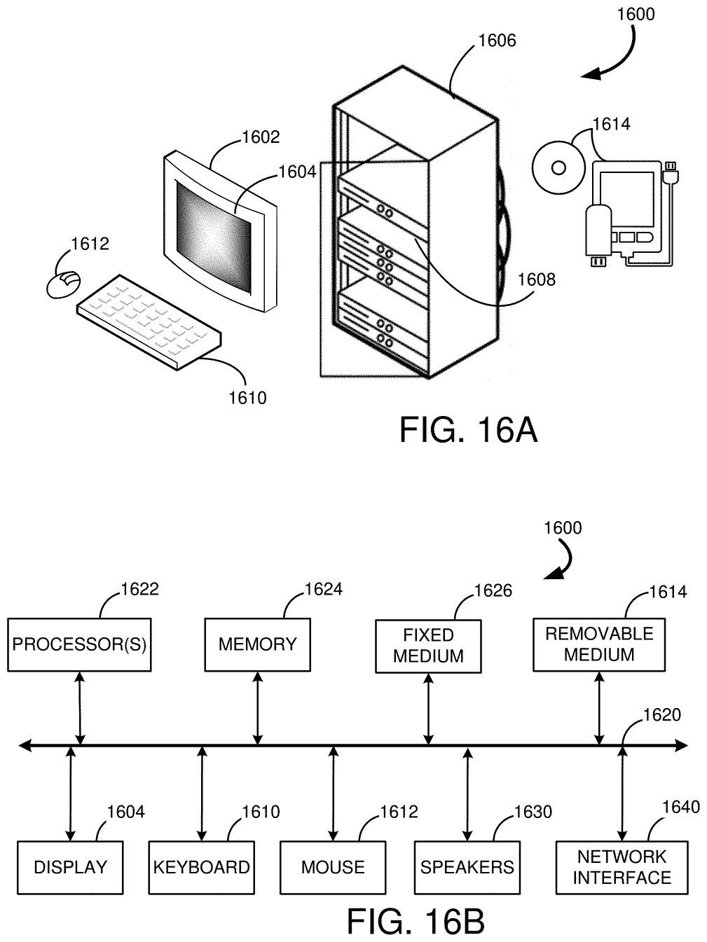

Now that the systems and methods for a personal medical monitoring device have been provided, attention shall now be focused upon apparatuses capable of executing the above functions in real-time. To facilitate this discussion, FIGS. 16A and 16B illustrate a Computer System 1600, which is suitable for implementing embodiments of the present invention. FIG. 16A shows one possible physical form of the Computer System 1600. Of course, the Computer System 1600 may have many physical forms ranging from a printed circuit board, an integrated circuit, and a small handheld device up to a huge super computer. Computer system 1600 may include a Monitor 1602, a Display 1604, a Housing 1606, server blades including one or more storage Drives 1608, a Keyboard 1610, and a Mouse 1612. Medium 1614 is a computer-readable medium used to transfer data to and from Computer System 1600.

FIG. 30B is an example of a block diagram for Computer System 1600. Attached to System Bus 1620 are a wide variety of subsystems. Processor(s) 1622 (also referred to as central processing units, or CPUs) are coupled to storage devices, including Memory 1624. Memory 1624 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable form of the computer-readable media described below. A Fixed Medium 1626 may also be coupled bi-directionally to the Processor 1622; it provides additional data storage capacity and may also include any of the computer-readable media described below. Fixed Medium 1626 may be used to store programs, data, and the like and is typically a secondary storage medium (such as a hard disk) that is slower than primary storage. It may be appreciated that the information retained within Fixed Medium 1626 may, in appropriate cases, be incorporated in standard fashion as virtual memory in Memory 1624. Removable Medium 1614 may take the form of any of the computer-readable media described below.

Processor 1622 is also coupled to a variety of input/output devices, such as Display 1604, Keyboard 1610, Mouse 1612 and Speakers 1630. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, motion sensors, brain wave readers, or other computers. Processor 1622 optionally may be coupled to another computer or telecommunications network using Network Interface 1640. With such a Network Interface 1640, it is contemplated that the Processor 1622 might receive information from the network, or might output information to the network in the course of performing the above-described intelligent payroll management. Furthermore, method embodiments of the present invention may execute solely upon Processor 1622 or may execute over a network such as the Internet in conjunction with a remote CPU that shares a portion of the processing.

Software is typically stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory in this disclosure. Even when software is moved to the memory for execution, the processor may typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

In operation, the Computer System 1600 can be controlled by operating system software that includes a file management system, such as a medium operating system. One example of operating system software with associated file management system software is the family of operating systems known as Windows® from Microsoft Corporation of Redmond, Wash., and their associated file management systems. Another example of operating system software with its associated file management system software is the Linux operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is, here and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments. The required structure for a variety of these systems may appear from the description below. In addition, the techniques are not described with reference to any particular programming language, and various embodiments may, thus, be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, Glasses with a processor, Headphones with a processor, Virtual Reality devices, a processor, distributed processors working together, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer (or distributed across computers), and when read and executed by one or more processing units or processors in a computer (or across computers), cause the computer(s) to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A handheld medical device comprising:
a housing including a front and rear side;
a diaphragm located in the center of the rear side of the housing;
at least three rear electrodes configured to collect electromagnetic signals from a patient, wherein the at least three rear electrodes are semi-circular in shape and encircle the diaphragm on the rear side of the housing;
at least two front electrodes located on the front side of the housing configured to collect electromagnetic signals from the patient, wherein a first of the front electrodes is located on the left lateral side of the housing and a second front electrode is located on the right lateral side of the housing; and
a screen located between the first and second front electrodes.

2. The medical device of claim 1, wherein the electrodes are brass plated by nickel which is then plated with gold.

3. The medical device of claim 1, further comprising a transmitter configured to pair the medical device with a user device operating an application.

4. The medical device of claim 3, wherein the transmitter is a Bluetooth module.

5. The medical device of claim 1, wherein the collected electromagnetic signals are combined to generate seven channel ECG signals.

6. The medical device of claim 5, wherein three of the rear electrodes are labeled as electrode 1 (E1) electrode 2 (E2) and electrode 3 (E3), and two of the front electrodes are labeled a left finger electrode (LA) and a right finger electrode (RA).

7. The medical device of claim 6, wherein the seven ECG channel signals are calculated as a first channel (CH1) generated by the LA minus RA (LA−RA), a second channel (CH2) generated by Electrode 2 minus RA (E2−RA), a third channel (CH3) generated by the second channel minus the first channel (CH2−CH1), a fourth channel (CH4) generated by a negative of the first channel plus the second channel divided by 2 (−(CH1+CH2)/2), a fifth channel (CH5) generated by the first channel minus the second channel divided by 2 (CH1−CH2/2), a sixth channel (CH6) generated by the second channel minus the first channel divided by 2 (CH2−CH1/2), and a seventh channel (CH7) generated by the Electrode 1 minus WTC (E1−WTC), where the WTC is the RA, LA and Electrode 2 signals added together and then divided by three ((RA+LA+E2)/3).

8. The medical device of claim 1, further comprising an optical sensor on the front side of the housing, below one of the front electrodes.

9. The medical device of claim 1, further comprising a temperature sensor on the front side of the housing below one of the front electrodes.

10. The medical device of claim 1, further comprising a second microphone.

11. The medical device of claim 10, wherein the diaphragm collects cardiac sound signals.

12. The medical device of claim 11, wherein the second microphone performs noise cancellation of the cardiac sound signals.

13. The medical device of claim 1, further comprising a backend server, wherein the backend server performs analysis of ECG and PCG signals collected by the medical device.

14. A method for generating seven channels of an electrocardiogram (ECG) from a five lead medical device comprising:
receiving five or less electrode signals, three or less from sensors, labeled E1, E2 and E3 respectively, located on the rear of a medical device, each of these rear electrodes in a semi-circular pattern around a diaphragm collecting electromagnetic signals from the chest region of a patient, and a first and a second front electrodes, labeled RA and LA, collecting signals from a right finger and left finger of the patient respectively; and
calculating at least seven ECG signals from the five or less collected signals.

15. The method of claim 14, wherein the at least seven ECG channel signals are calculated as a first channel (CH1) generated by the LA minus RA (LA−RA), a second channel (CH2) generated by Electrode 2 minus RA (E2−RA), a third channel (CH3) generated by the second channel minus the first channel (CH2−CH1), a fourth channel (CH4) generated by a negative of the first channel plus the second channel divided by 2 (−(CH1+CH2)/2), a fifth channel (CH5) generated by the first channel minus the second channel divided by 2 (CH1−CH2/2), a sixth channel (CH6) generated by the second channel minus the first channel divided by 2 (CH2−CH1/2), and a seventh channel (CH7) generated by the Electrode 1 minus WTC (E1−WTC), where the WTC is the RA, LA and Electrode 2 signals added together and then divided by three ((RA+LA+E2)/3).

16. The method of claim 14, further comprising collecting cardiac sound signals to generate a phonocardiogram (PCG).

17. The method of claim 16, further comprising collecting ambient noise using a second microphone, and adjusting the PCG by noise cancellation using the ambient noise.

18. The method of claim 14, wherein the left finger and the right finger are index fingers.

19. The method of claim 14, further comprising collecting at least one of an optical signal, a temperature signal, a skin conductivity signal, and one or more chemical signals.

20. The method of claim 19, wherein the optical signal is used to calculate at least one of blood pressure, pulse rate, glucose level, red blood cell count, and oxygenation of the patient's blood.
